(19) United States
(12) Patent Application Publication
(10) Pub. No.: US 2023/0233617 A1
(43) Pub. Date: Jul. 27, 2023

(54) METHODS FOR DIFFERENTIATING STEM CELLS INTO DOPAMINERGIC PROGENITOR CELLS

(71) Applicant: JZ CELL TECHNOLOGIES AB, Hässelby (SE)

(72) Inventors: Lars Johan Ericson, Solna (SE); Zjanna Vladimirovna Alekseenko, Solna (SE)

(21) Appl. No.: 17/923,265

(22) PCT Filed: May 7, 2021
(86) PCT No.: PCT/EP2021/062226

§ 371 Date: Nov. 4, 2022

(30) Foreign Application Priority Data
May 7, 2020 (GB) 2006792.2

(51) Int. Cl. A61K 35/30; C12N 5/0793; A61P 25/16

(52) U.S. Cl. CPC A61K 35/30; C12N 5/0619; A61P 25/16; C12N 2506/02; C12N 2533/52; C12N 2509/00; C12N 2501/13; C12N 2501/727; C12N 2500/38

(57) ABSTRACT

The present invention relates to methods for differentiating stem cells into ventral midbrain dopaminergic progenitor cells, and into mesencephalic dopaminergic neurons, and compositions, kits, and uses thereof.

The present invention relates to methods for differentiating stem cells into ventral midbrain dopaminergic progenitor cells, and into mesencephalic dopaminergic neurons, and compositions, kits, and uses thereof.

Neurodegenerative disorders such as Parkinson's, Alzheimer's, and Huntington's disease are incurable and debilitating conditions that result in progressive degeneration and/or death of neuronal populations. Parkinson's disease (PD) is associated with the progressive loss of dopaminergic (DA) neurons in the substantia nigra pars compacta (SNpc), a part of the midbrain, and therefore mainly affects the motor system leading to bradykinesia, rigidity, and resting tremor.

Treating neurodegenerative diseases with cell transplantation began with clinical trials in the late 1980s in which dopamine neuron progenitor cells from the foetal brain were transplanted into individuals with Parkinson's disease. These trials demonstrated that grafted cells can restore lost dopamine neurotransmission and reverse motor deficits (Björklund, A. & Lindvall, O. J. Parkinsons. Dis. 7, S21-S31 (2017)). However, the use of foetal tissue is associated with a number of difficulties, such as low availability and high variability, and numerous ethical concerns. Following the derivation of human embryonic stem cells (hESCs), and the discovery of induced pluripotent stem cells (iPSCs) in 2007, a new scalable source of human pluripotent stem cells (hPSCs) that could potentially replace foetal tissue became available.

Extensive developments have been made to try to control the differentiation of pluripotent stem cells into midbrain dopaminergic (mDA) neurons (Kirkeby, A. et al. Cell Rep. 1, 703-714 (2012), Kriks, S. et al. Nature 480, 547-551 (2011), and Marton, R. M. & loannidis, J. P. A. Stem Cells Transl. Med. 8, 366-374 (2019)). Grafted hPSC-derived preparations show functional efficacy in animal PD models, and clinical trials using allogenic ESCs or autologous iPSCs as starting material have either been initiated or are scheduled for the near future (Barker, R. A., Parmar, M., Studer, L. & Takahashi, J. Cell Stem Cell 21, 569-573 (2017)).

Despite these advances, there is a continuous need to enhance robustness of differentiation protocols in order to increase consistency and minimize batch-to-batch adjustments when new hPSC-lines are taken into use (Nolbrant, S., Heuer, A., Parmar, M. & Kirkeby, A. Nat. Protoc. 12, 1962-1979 (2017). This is particularly important when patient-specific autologous iPSC-lines or large numbers of human leukocyte antigen (HLA) matched donor iPSCs are considered for routine clinical practice, a likely progress as immunologically matched iPSCs are favorable over allogenic ESC-lines from an immunological perspective (Wang, S. et al. Cell Discov. 1, 1-11 (2015) and Morizane, A. et al. Nat. Commun. 8, 1-12 (2017)).

The extended time required to produce hPSC-derived functional human neurons in culture provides another challenge and cost burden that hampers routine application of hPSC-derived cells in disease modeling or high-throughput drug development (Qi, Y. et al. Nat. Biotechnol. 35, 154-163 (2017)). Protocols for generating mDA neurons have been progressively improved with respect to yield of desired cell type, but the time to obtain mature mDA neurons in culture have remained essentially constant since the first hPSC-based protocol was described in 2004 (Marton, R. M. & loannidis, J. P. A. (2019)). It has been reported to take (typically takes) 60 days or more to generate mature human mDA neurons which exhibit the required electrophysiological characteristics in culture (Niclis, J. C. et al. Stem Cells Transl. Med. 6, 937-948 (2017) and Riessland, M. et al. Cell Stem Cell 25, 514-530.e8 (2019)) which could reflect the minimal time required for cells to reach a functional state. However, single cell analyses suggest slower kinetics and less tightly controlled developmental progression of hPSC-derived mDA neurons relative to their in vivo counterpart (La Manno, G. et al. Cell 167, 566-580.e19 (2016)) raising the possibility that current methods have not been optimized regarding timing of differentiation.

Current mDA neuron protocols utilize timed activation of WNT signaling, or of WNT and FGF signaling, to specify midbrain (MB) character by mimicking the patterning activity of WNT1 and FGF8 produced by the isthmic organizer at the boundary between the MB and hindbrain (HB) (Tao, Y. & Zhang, S.-C. Cell Stem Cell 19, 573-586 (2016)). The glycogen synthase kinase 3β (GSK3β) inhibitor CHIR99021 is applied to activate the WNT pathway, but the specification of anteroposterior (AP) identity by CHIR99021 is highly concentration-sensitive (Lu, J; Zhong, et al. S. Nat. Biotechnol. 34, 89-95 (2015)). This impinges on consistency and entails very careful titrations for individual hPSC-lines. Also, assessment of a large set of CHIR99021-based transplantation experiments in a rat PD model revealed significant inter-experimental variability and that poor graft outcome correlated with expression of diencephalic genes in preparations prior to transplantation, suggesting imprecise regional specification of cells even after optimized titration of CHIR99021 (Kirkeby, A. et al. Cell Stem Cell 20, 135-148 (2017)).

Therefore, alternative methods are needed for more efficient derivation of mDA neurons from stem cells.

Against this background, the inventors have surprisingly discovered a novel retinoic acid (RA)-based method for robust and fast derivation of human mDA neurons at high-yield. Unlike other methods of differentiating hPSCs into dopaminergic neurons, which can be lengthy, variable, and require extensive protocol-adjustments for individual hPSC-lines (and produce a biologically irrelevant phenotype), the present invention allows stem cells to be robustly differentiated into ventral midbrain dopaminergic progenitor cells which could then be used for transplantation, or further differentiated into mature authentic midbrain dopaminergic neurons with increased speed and unprecedented scalability, all while retaining proper midbrain phenotype throughout.

In one aspect, the present invention provides a method for differentiating stem cells into ventral midbrain dopaminergic progenitor cells, the method comprising contacting a plurality of stem cells with an effective amount of at least one activator of retinoic acid (RA) signalling, and culturing the stem cells under conditions sufficient to cause differentiation of the stem cells into a cell population comprising ventral midbrain dopaminergic progenitor cells.

By “stem cells” we include cells found in embryonic and adult tissues that have the ability to self-renew and differentiate into different cell types. Stem cells are classified as totipotent, pluripotent, multipotent, or unipotent depending on their potential to generate the variety of cell lineages. Preferred stem cells in the context of the present invention are discussed below.

The “plurality of stem cells” of the present method may comprise at least or about 10⁴, 10⁵, 10⁶, 10⁷, 10⁸, 10⁹, 10¹⁰, 10¹¹, 10¹², 10¹³ cells or any range derivable therein. The starting plurality of stem cells may have a seeding density of at least or about 10, 10¹, 10², 10³, 10⁴, 10⁵, 10⁶, 10⁷, 10⁸ cells/mL, or any range derivable therein. In an embodiment, the plurality of stem cells are plated at a density of 60,000-80,000 cells/cm².

By “differentiating” and “differentiation” we include a process whereby an unspecialised, or less specialised (uncommitted) stem cell, such as a pluripotent stem (PS) cell or an induced pluripotent stem (iPS) cell, acquires phenotypic features of a specialised cell (a terminally differentiated cell) with specific purpose and functions, such as a neural cell. Differentiation of a stem cell may be determined by methods well known in the art, including analysis of cell markers or morphological features associated with cells of a defined differentiated state.

Thus “differentiating stem cells”, in the context of the present invention, includes inducing the stem cell to produce cells with characteristics that are different from the stem cell, such as transcriptome and/or phenotype (i.e. change in expression of a protein, such as forkhead box protein A2 (FOXA2) or a set of proteins, such as forkhead box protein A2 (FOXA2) and LIM homeobox transcription factor 1 alpha (LMX1A)).

By the term “midbrain (MB)”, also known as the “mesencephalon”, we include a region of the developing vertebrate brain between the forebrain (FB) (anterior) and the hindbrain (HB) (posterior). Midbrain includes the tectum, tegmentum, and substantia nigra (SN), and is composed by many molecularly and functionally distinct types of neurons. The midbrain serves important functions in motor movement, particularly movements of the eye, and in auditory and visual processing.

As used herein, the terms “dorsal” and “ventral” are used as anatomical terms of location in the animal body, herein dorsal refers to the “back end” of the body and ventral refers to the “front end”. The term “dorsoventral axis”, “dorso-ventral axis” or “D-V axis” refers to the imaginary line obtained by connecting these points. As used herein, the terms “anterior”, “posterior”, “rostral” and “caudal” are used as anatomical terms of location in the animal body, wherein anterior refers to the “head end” of the body, and posterior refers to the polar opposite of anterior (the “tail end”). The terms “anterior” and “rostral” are used interchangeably and the terms “posterior” and “caudal” are used interchangeably. The term “anteroposterior axis”, “anterior-posterior axis”, “antero-posterior axis” or “A-P axis” refers to an imaginary line connecting these two points.

By “dopaminergic” (DA) neurons we include a collection of neurons in the central nervous system that synthesize the neurotransmitter dopamine (DA). Midbrain or mesencephalic dopaminergic neurons (mDA) are developmentally partitioned to three distinct nuclei: (i) the substantia nigra pars compacta (A9 group), which is primarily affected in Parkinson's disease, (ii) the ventral tegmental area (A10 group), and (iii) the retrorubral field (A8 group). SNpc and VTA DA neurons represent two of the nine major DA neuron groups in the mammalian brain as identified by staining for tyrosine hydroxylase (TH), the enzyme that catalyses the rate-limiting step in the synthesis of dopamine.

A unique feature of mDA neurons is that they originate from initially non-neuronal floor plate (FP) cells at the ventral midline of the MB, and progenitors must acquire neuronal potential prior to differentiation into neurons.

By “progenitor cells” we include partially differentiated cells. The terms “progenitor cells” and “progenitors” may be used interchangeably herein. The progenitor cells have the capacity to differentiate into a variety of neural subtypes; particularly a variety of dopaminergic neuronal subtypes, upon culturing the appropriate factors, such as those described herein. In the context of the present invention, the progenitor cells are neural progenitors, specifically ventral midbrain dopaminergic progenitor cells, primed to differentiate into DA neurons, such as A9 or A10 neurons.

It will be understood that the non-stem cell progeny of neural stem cells (NSCs) are referred to as neural progenitor cells. Neural progenitor cells have the capacity to proliferate and differentiate into more than one neuronal cell type. A distinguishing feature of a neural progenitor cell is that, unlike a stem cell, it has a limited proliferative ability and does not exhibit self-renewal.

During foetal development, progenitor cells of DA neurons are formed in the ventral neural tube of the developing mesencephalon. Progenitor cells from the so-called floor plate region are characterized by expression of the transcription factors including LMX1A, FOXA2, and OTX2. These cells give rise to DA SNpc neurons (A9 group) and to DA VTA neurons (A10 group). These progenitor cells are termed “ventral midbrain dopaminergic progenitor cells” as used herein. Ventral midbrain dopaminergic progenitor cells do not express NKX2.1, BARHL1, BARHL2, PITX2, NKX2.2, PHOX2B, PHOX2A and NKX6.1 either alone or in combination which instead define progenitors giving rise to subthalamic neurons, GABAergic midbrain neurons, cranial motor neurons (MNs) and serotonergic neurons (5HTNs) in the ventral HB. Ventral midbrain dopaminergic progenitor cells have the capacity to differentiate into mature functional dopaminergic (DA) neurons.

Ventral midbrain progenitors can be distinguished from diencephalic subthalamic neuron progenitors, which also express LMX1A, FOXA2 and OTX2. However, diencephalic subthalamic neuron progenitors also express BARHL1, BARHL2, PITX2 and NKX2.1 which distinguish them from ventral midbrain progenitors.

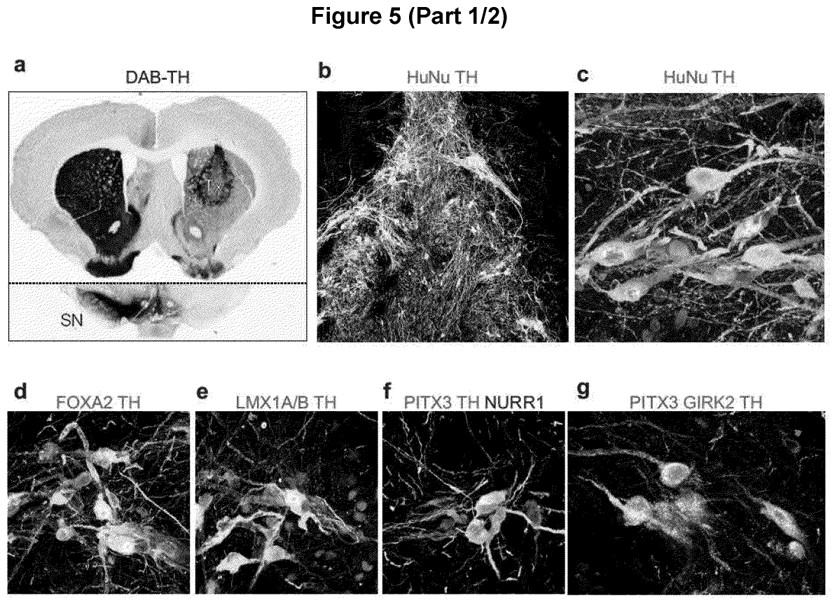

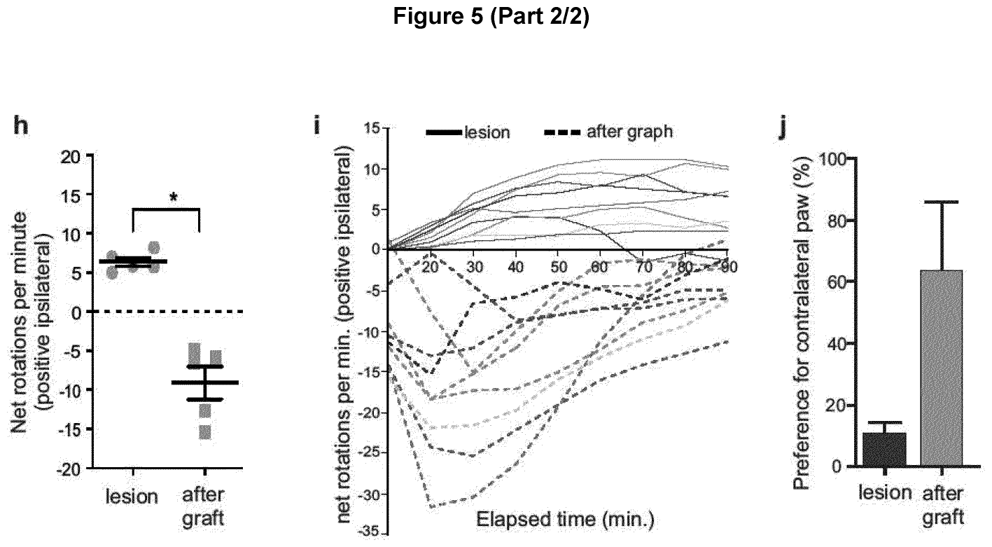

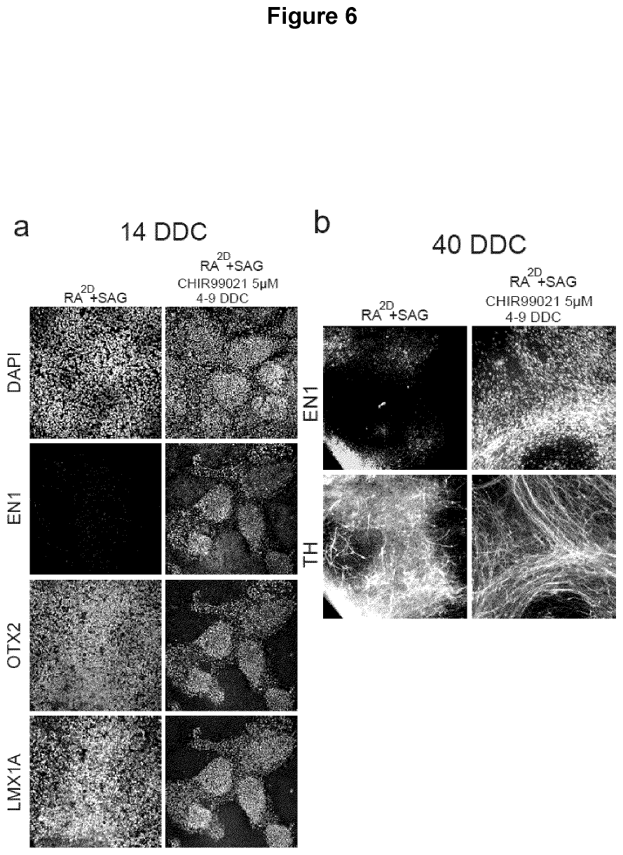

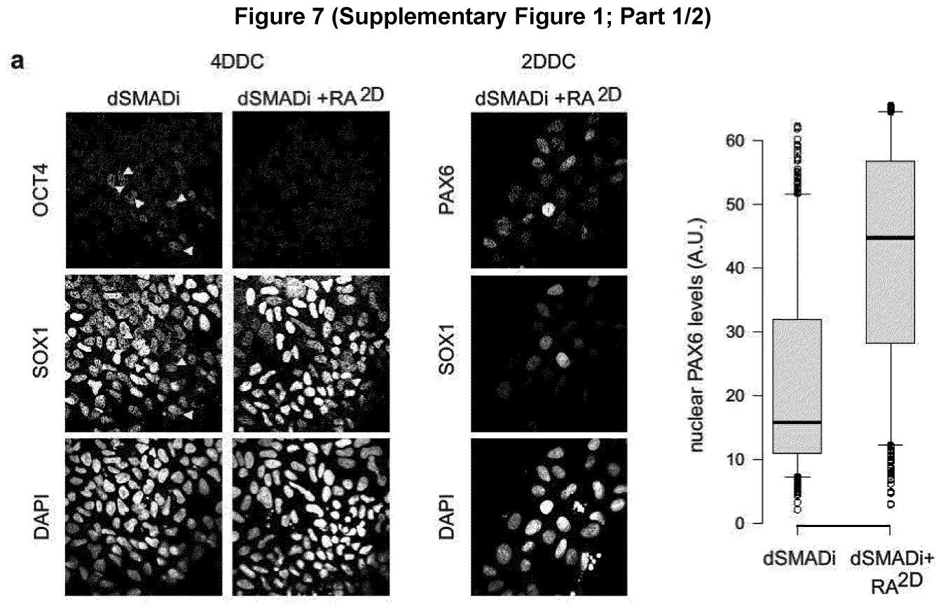

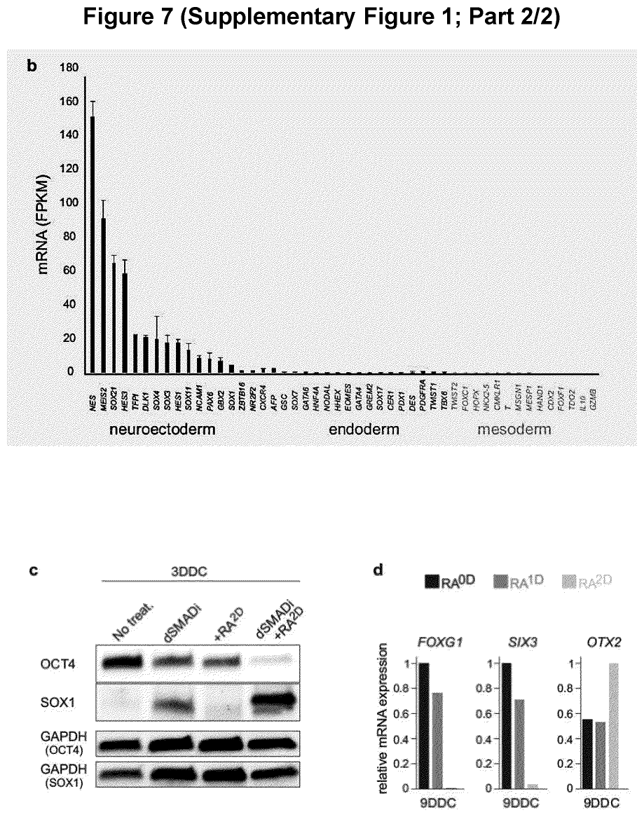

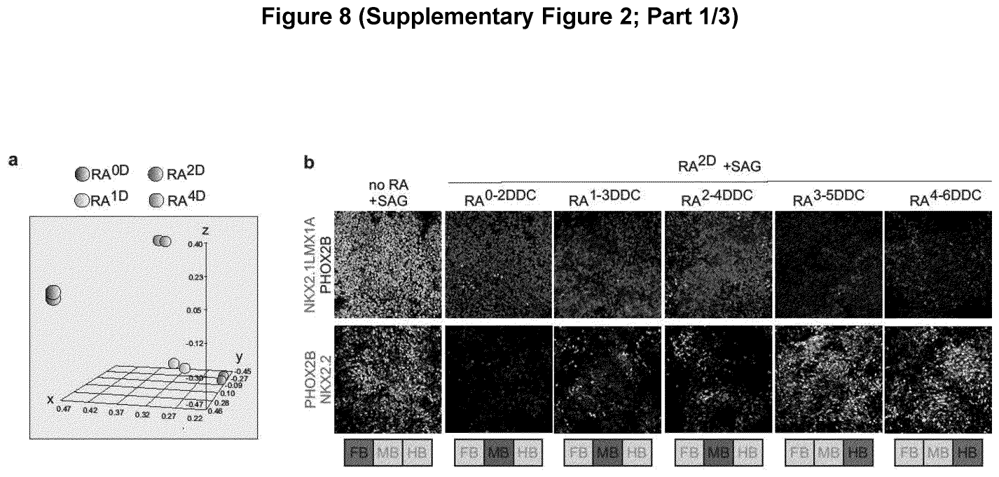

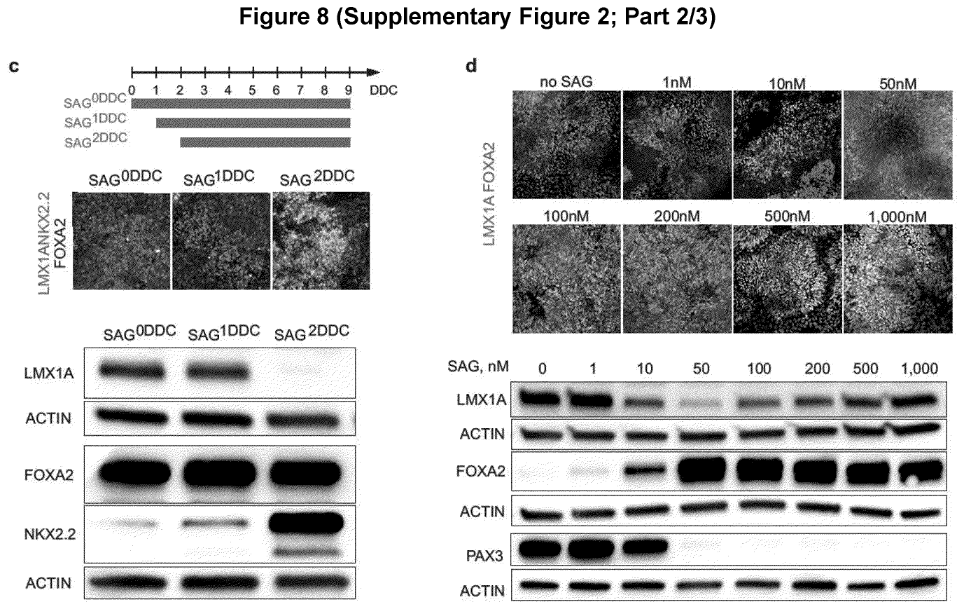

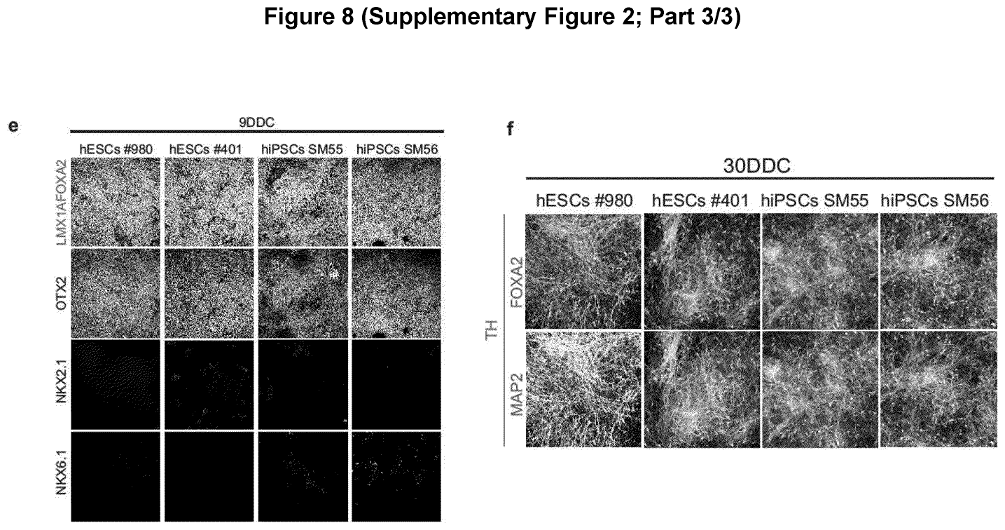

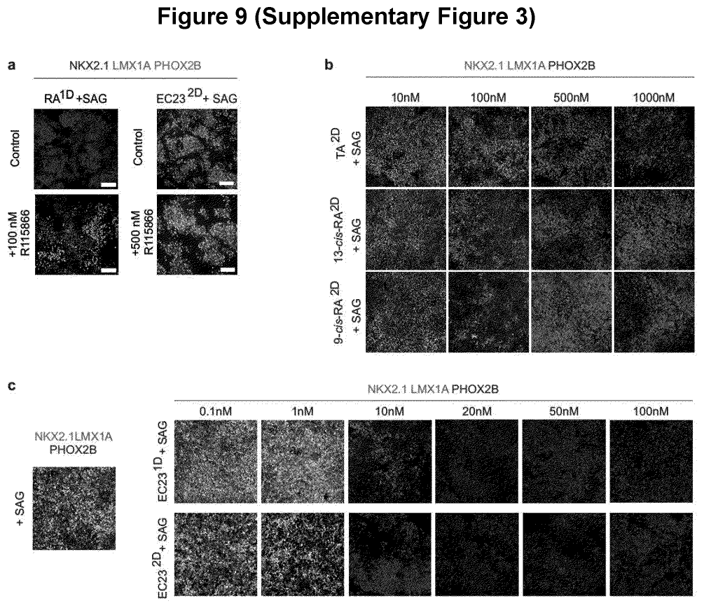

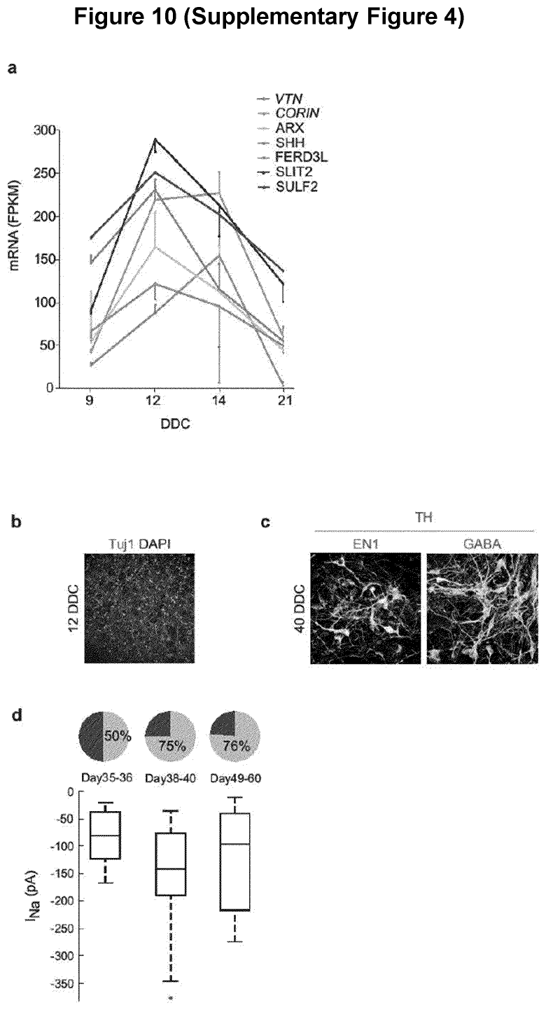

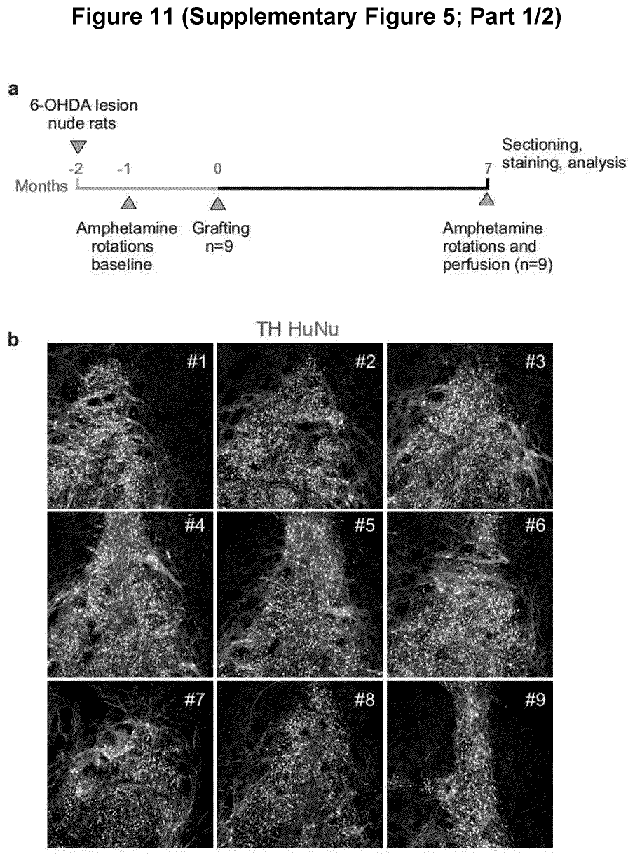

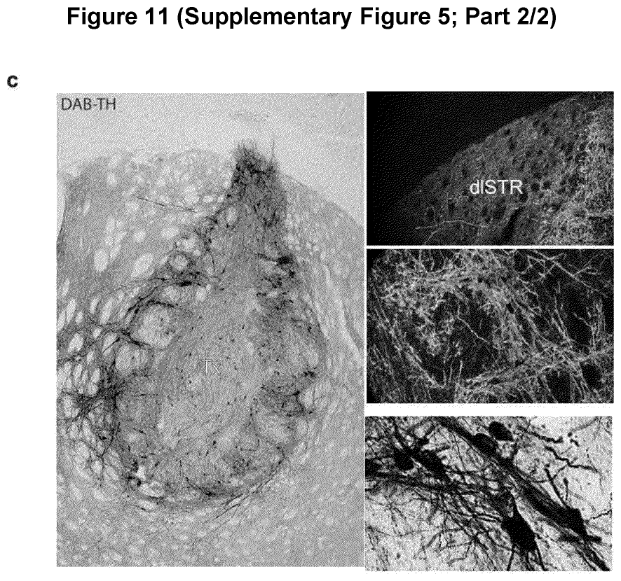

The inventors have shown that these correctly patterned progenitor cells give rise to mature and functional mesencephalic DA neurons upon transplantation into adult rats (See Examples and FIG. 5a-c and FIG. 11b (Supplementary FIG. 5b)).

By “contacting” cells with a compound (e.g. one or more inhibitor, activator, and/or inducer), we include providing the compound in physical proximity with the cells in order to produce (obtain) “contacted” cells, in other word providing the compound in a location that permits the cell or cells access to the compound. The contacting may be accomplished using any suitable method. For example, contacting can be accomplished by adding the compound, in concentrated form, to a cell or population of cells, for example in the context of a cell culture, to achieve the desired concentration. Contacting may also be accomplished by including the compound as a component of a formulated culture medium.

By “effective amount” we include a quantity sufficient to achieve a desired physiological and/or therapeutic effect. In the context of methods for the differentiation of stem cells into ventral midbrain dopaminergic progenitor cells, an effective amount of a substance is any amount of the substance which can specify a midbrain identity to neural stem cells and/or an amount sufficient to direct the fate of pluripotent stem cells towards dopaminergic neurons having midbrain identity. Methods of determining an “effective amount” are well known to those skilled in the art and typically involve titrating the dose of the substance(s) until the desired effect is achieved.

By “neural stem cell (NSC)” we include multipotent cells which are able to proliferate and self-renew, and to produce progeny cells which terminally differentiate into the three major cellular types of the central nervous system: neurons, astrocytes, and oligodendrocytes. In contrast, neural progenitor cells have a more restricted developmental potential and limited proliferative capacity. In addition, they can only differentiate into a more restricted variety of cell types (i.e. cells that have already become lineage committed to give rise to only one category of neural component, e.g., glial cells versus neurons).

By “activator,” we include compounds that increase, induce, stimulate, activate, facilitate, or enhance activation the signalling function of the molecule or pathway, e.g., RA signalling, etc. An activator may enhance or increase the pathway to be activated by 10%, 20%, 30%, 40%, 50%, 60%, 70%, 80%, 90%, 100% or more when compared to the activity of the pathway without or before the addition of the activator.

It will be appreciated that the term “activator of retinoic acid (RA) signalling” as used herein includes any compound or molecule capable of potentiating or substituting retinoic acid (RA) signalling. Such activators may be involved in signalling downstream or upstream of retinoic acid or be small molecule agonists of the signalling pathway. A non-limiting list of activators of retinoic acid (RA) signalling, including retinoic acid derivatives or agonists thereof is provided herein.

Retinoic acid and its derivatives, particularly 9-cis-retinoic acid (9cRA), 13-cis-retinoic acid (13cRA), and all-trans-retinoic acid (ATRA), are a group of structurally simple lipid molecules derived from vitamin A (retinol) that transactivate numerous genes and exert pleiotropic effects on cellular growth, differentiation and homeostasis both in vivo and in vitro in all vertebrates. They modulate the expression of their target genes by binding to two classes of nuclear receptors, retinoic acid receptors (RAR) and retinoid X receptors (RXR). ATRA and 13cRA can only bind efficiently to RAR, but 9cRA is a ligand for both nuclear receptors RAR and RXR. Any molecule that can mimic the effect of retinoic acid is contemplated in the present invention. The skilled person could determine if a molecule was an activator of retinoic acid (RA) signalling, for example, by analysis of target genes of RA expression by immunocytochemistry, qPCR, immunoblotting, RNA-seq or other biochemical techniques known in the art. Target genes could include CYP26A1, RARA, RARB, MEIS2, HOXA1, CRABP1, CRABP2.

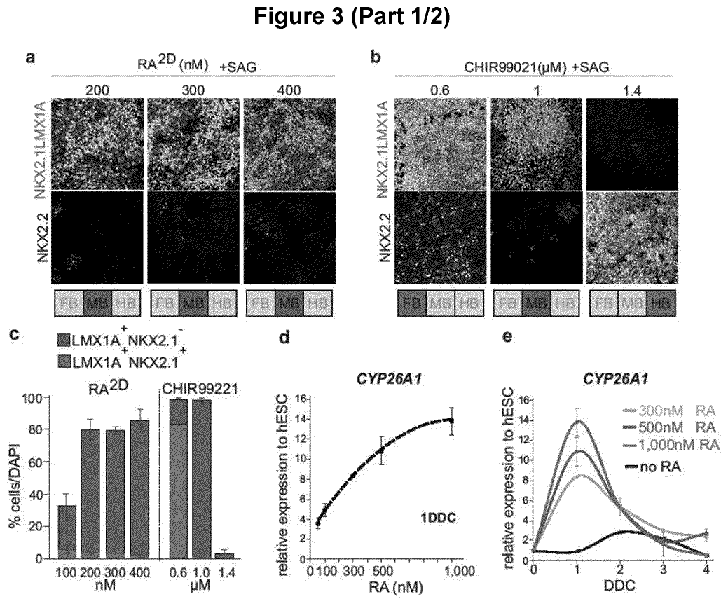

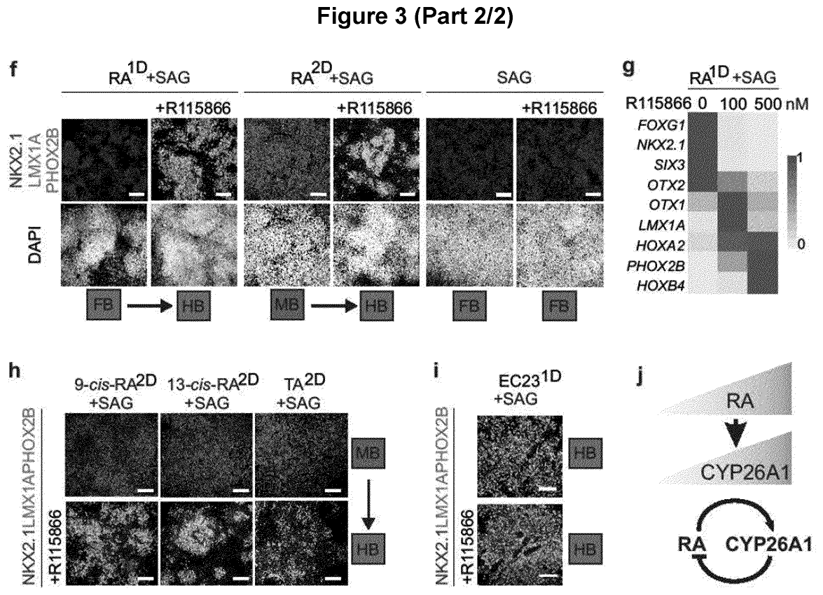

In one embodiment, the effective amount of the at least one activator of retinoic acid (RA) signalling is an amount sufficient to provide a final concentration in the culture media of about 10-800 nM. In a particular embodiment the effective amount of the at least one activator of retinoic acid (RA) signalling is an amount sufficient to provide a final concentration in the culture media of about 100-800 nM. In a particular embodiment the effective amount of the at least one activator of retinoic acid (RA) signalling is an amount sufficient to provide a final concentration in the culture media of about 200-800 nM. In a particular embodiment the effective amount of the at least one activator of retinoic acid (RA) signalling is an amount sufficient to provide a final concentration in the culture media of about 200-500 nM. In a particular embodiment the effective amount of the at least one activator of retinoic acid (RA) signalling is an amount sufficient to provide a final concentration in the culture media of about 200-400 nM. In a particular embodiment the effective amount of the at least one activator of retinoic acid (RA) signalling is an amount sufficient to provide a final concentration in the culture media of about 300 nM. Activators depending on their nature could work in a different range of concentrations. For example, a less potent factor may show an effect only in μM concentrations. Conversely, the RA-analogues EC23 that cannot be degraded by CYP26 enzymes can impose a vMB-identity to stem cells at low concentrations (10 nM), as exemplified in FIG. 9c (supplementary FIG. 3c).

It will be appreciated that the “effective amount of at least one activator of retinoic acid (RA) signalling” may vary depending on the identity of the activator of RA signalling. For example, when the activator of RA signalling is ATRA the effective amount of retinoic acid may be an amount sufficient to provide a final concentration in the culture media of about 100-800 nM. In a particular embodiment the effective amount of ATRA may be an amount sufficient to provide a final concentration in the culture media of about 200-500 nM. In a particular embodiment the effective amount of ATRA may be an amount sufficient to provide a final concentration in the culture media of about 200-400 nM. In a particular embodiment the effective amount of ATRA may be an amount sufficient to provide a final concentration in the culture media of about 300 nM.

When the activator of RA signalling is a molecule other than ATRA, it will be appreciated that the effective amount of that molecule will be an amount that gives rise the same effect (i.e. specifying midbrain identity) as the effective amount of ATRA (e.g. any of the effective amounts described in the immediately preceding paragraph). For example, when the activator of RA signalling is 13-cis-RA, the effective amount of 13-cis-RA will be the amount of 13-cis-RA that gives rise to the same effect (i.e. specifying midbrain identity) as an effective amount of ATRA (e.g. any of the effective amounts described in the immediately preceding paragraph). As demonstrated by the inventors in the Examples exposure of cells to 500 nM of analogues of ATRA (9-cis RA, 13-cis RA and the xenobiotic RA-analogue tazarotenic acid (TA)) for 48-hours mimicked the patterning activity of ATRA by imposing a LMX1A+/NKX2.1− vMB identity (FIG. 3h and FIG. 9b (Supplementary FIG. 3b)).

Unlike ATRA, 9-cis RA, 13-cis RA, and tazarotenic acid (TA), the synthetic RA analogue EC23 is more stable because it is resistant to CYP26-mediated oxidation (Lopez-Real, R. E. et al. J. Anat. 224, 392-411 (2014)). Therefore, when cells were exposed to 200 nM of EC23, the cells acquired a hindbrain identity. Titration experiments showed that EC23 could differentiate pluripotent stem cells into ventral midbrain dopaminergic progenitor cells, but this required a ˜20-fold reduction in concentration and treatment of cells only for 24 hours (FIG. 9c (Supplementary FIG. 3c)). Accordingly, a person skilled in the art person can carry out the titration experiments described herein, or otherwise known in the art, to determine the effective amount of any given activator of RA signalling needed to differentiate stem cells into ventral midbrain dopaminergic progenitor cells.

As used herein, the term “population” refers to a group of cells. In particular embodiments, said group of cells may comprise at least two cells, such as at least or about 10⁴, 10⁵, 10⁶, 10⁷, 10⁸, 10⁹, 10¹⁰, 10¹¹, 10¹², 10¹³.

The population may be a pure population comprising one cell type, such as a population of neuronal cells or a population of undifferentiated pluripotent stem cells. Alternatively, the population may comprise more than one cell type, for example a mixed cell population.

By “culturing the stem cells under conditions sufficient to cause differentiation of the stem cells into a cell population comprising ventral midbrain dopaminergic progenitor cells”, we include the meaning of culturing the stem cells under conditions in which they can differentiate into ventral midbrain dopaminergic progenitor cells in the presence of the at least one activator of RA signalling. Any suitable conditions may be used. For example, the stem cells may be exposed to one or more agents and/or environmental conditions, which direct differentiation of the plurality of stem cells into ventral midbrain dopaminergic progenitor cells, when the cells are in the presence of the at least one activator of RA signalling. Also included are conditions necessary to promote cell viability. Such conditions are well known in the art of culturing stem cells, and it will be appreciated that the skilled person would be able to select appropriate conditions for a given stem cell type.

Such culture conditions may include those that mimic the graded patterning signals that impose unique regional identities of NSCs along the anteroposterior (AP) and dorsoventral (DV) axes of the neural tube. For example, such conditions may include agents capable of imposing ventral regional specification on stem cells. This may be achieved by one or more agents which activate hedgehog pathway signalling.

Distinct types of neurons are generated at different positions of the neural tube in response to graded patterning signals that impose unique regional identities of NSCs along the anteroposterior (AP) and dorsoventral (DV) axes of the neural tube. In neural development and in hPSC-cultures, NSCs acquire a cortical forebrain (FB) identity by default in the absence of patterning signals. WNT, FGF and retinoic acid (RA) are the three major signalling pathways implicated in imposing more posterior midbrain (MB), hindbrain (HB) or spinal cord (SC) character of NSCs. Graded Sonic hedgehog (SHH) and BMP signalling, in turn, impose distinct identities of NSCs along the DV axis of the neural tube. mDA neuron progenitor cells defined by their co-expression of the transcription factors LMX1A, LMX1B, OTX2 and FOXA2 are localized at the ventral midline of the developing MB. Current hPSC-based mDA neuron protocols are technically related and utilize timed activation of WNT signalling, or a combination of WNT and FGF signalling, to specify MB character and SHH signalling to induce a ventral mDA neuron progenitor identity of NSCs. The application of WNT and FGF in these protocols are applied to try to mimic WNT1 and FGF8 signalling by the isthmus, which is a secondary organizer centre established at the boundary between the MB and HB.

The role for RA signaling in patterning of the HB and spinal cord is well-established, and RA signaling is commonly used in hPSC-based protocols for production of neurons with a caudal origin in the neural tube, such as somatic motor neurons. In developing embryos, a posterior-to-anterior gradient of RA signaling is believed to reach rostral parts of the HB but not into more rostral regions fated to become MB or FB. Due to the strong caudalizing effect of RA, it is assumed that RA signaling is incompatible with production of neurons with a rostral origin, including mDA neurons. In fact, many current state-of-the-art hPSC-based mDA neuron protocols therefore actively exclude RA, or vitamin A which is a precursor of RA, in their respective differentiation procedures (Kirkeby et al., 2017; Nolbrant et al., 2017; Monzel et al., 2017; Jovanic et al., 2018; Lehnen et al., 2017).

In a preferred embodiment, the at least one activator of retinoic acid (RA) signalling is effective to specify ventral midbrain identity to neural stem cells.

For example, the at least one activator is effective to specify ventral midbrain identity to neural stem cells when under conditions sufficient to cause differentiation of the stem cells into a cell population comprising ventral midbrain dopaminergic progenitor cells.

By the term “ventral midbrain identity” as used herein we include that the neural progenitor cells in vitro express markers specific to midbrain and do not express markers specific to the other regional progenitor cells of the brain (i.e. forebrain or hindbrain). Ventral midbrain progenitor cells express LMX1A, LMX1B, FOXA2, OTX2, and do not express NKX2.1, NKX2.2, BARHL1, BARHL2, PITX2, NKX6.1, PHOX2B, PHOX2A, FOXG1, EMX2, PAX6, SIX3, SIX6, LHX2, HOXA2, HOXB4.

In an embodiment, at least 60%, 65%, 70%, 75%, 80%, 85%, 90%, 95%, 96%, 97%, 98% or 99% of the cells in the cell population comprising ventral midbrain dopaminergic progenitor cells are positive for a marker of ventral midbrain dopaminergic progenitor cells. The term “marker”, as used herein, refers to nucleic acid or polypeptide molecules that are differentially expressed in a cell of interest. In the context of the present invention, differential expression means an increased level for a positive marker and a decreased level for a negative marker as compared to an undifferentiated cell. The detectable level of the marker nucleic acid or polypeptide is sufficiently higher or lower in the cells of interest compared to other cells, such that the cell of interest can be identified and distinguished from other cells. A variety of methods for observing and quantitating marker expression are known in the art and include immunocytochemistry and immunohistochemistry (see Examples) and immunoblotting, qPCR and RNA sequencing.

As used herein, a cell is “positive for” a specific marker, or “positive”, when the specific marker is detected in the cell. Conversely, the cell is “negative for” a specific marker, or “negative”, when the specific marker is not detected in the cell. The use of “+” or “−” signs in connection with a marker is herein meant to be understood as positive or negative for said marker (for example LMX1A+ cells are positive for the marker LMX1A).

In a particular embodiment, the ventral midbrain dopaminergic progenitor cells express forkhead box protein A2 (FOXA2) and LIM homeobox transcription factor 1 alpha (LMX1A).

“FOXA2” is a protein that in humans is encoded by the FOXA2 gene. Forkhead box protein A2 is a member of the forkhead class of DNA-binding proteins. FOXA2 can comprise a protein sequence such as depicted by Uniprot No. Q9Y261. The term FOXA2 encompasses any FOXA2 nucleic acid molecule or polypeptide and can also comprise fragments or variants thereof. The skilled person knows how to detect FOXA2. Such methods are also described in the Examples.

LIM homeobox transcription factor 1 alpha (LMX1A) is a protein that in humans is encoded by the LMX1A gene. LMX1 is a LIM homeobox transcription factor that binds an A/T-rich sequence in the insulin promoter and stimulates transcription of insulin. LMX1A can comprise a protein sequence such as depicted by Uniprot No. Q8TE12. The term LMX1A encompasses any LMX1A nucleic acid molecule or polypeptide and can also comprise fragments or variants thereof. The skilled person knows how to detect LMX1A. Such methods are also described in the Examples.

In a preferred embodiment, the ventral midbrain dopaminergic progenitor cells additionally express LIM homeobox transcription factor 1 beta (LMX1B) and Orthodenticle homeobox 2 (OTX2).

LIM homeobox transcription factor 1 beta (LMX1B) is a protein that in humans is encoded by the LMX1B gene. LMX1B is a LIM homeobox transcription factor which plays a central role in dorso-ventral patterning of the vertebrate limb. LMX1B can comprise a protein sequence such as depicted by Uniprot No. 060663. The term LMX1B encompasses any LMX1B nucleic acid molecule or polypeptide and can also comprise fragments or variants thereof. The skilled person knows how to detect LMX1 B. Such methods are also described in the Examples.

Orthodenticle homeobox 2 (OTX2) is a protein that in humans is encoded by the OTX2 gene. OTX2 can comprise a protein sequence such as depicted by Uniprot No. P32243. The term OTX2 encompasses any OTX2 nucleic acid molecule or polypeptide and can also comprise fragments or variants thereof. The skilled person knows how to detect OTX2 B. Such methods are also described in the Examples.

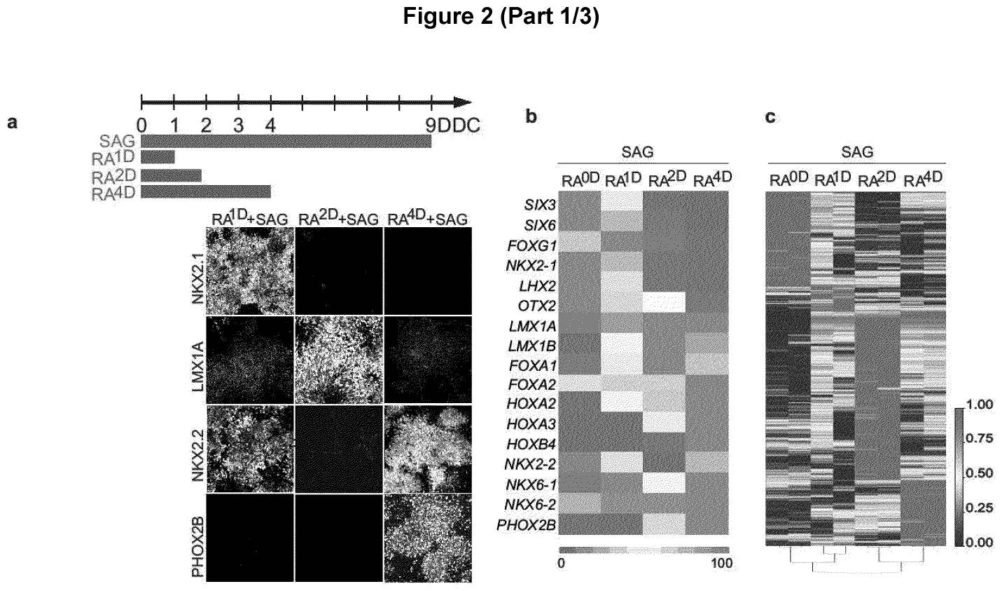

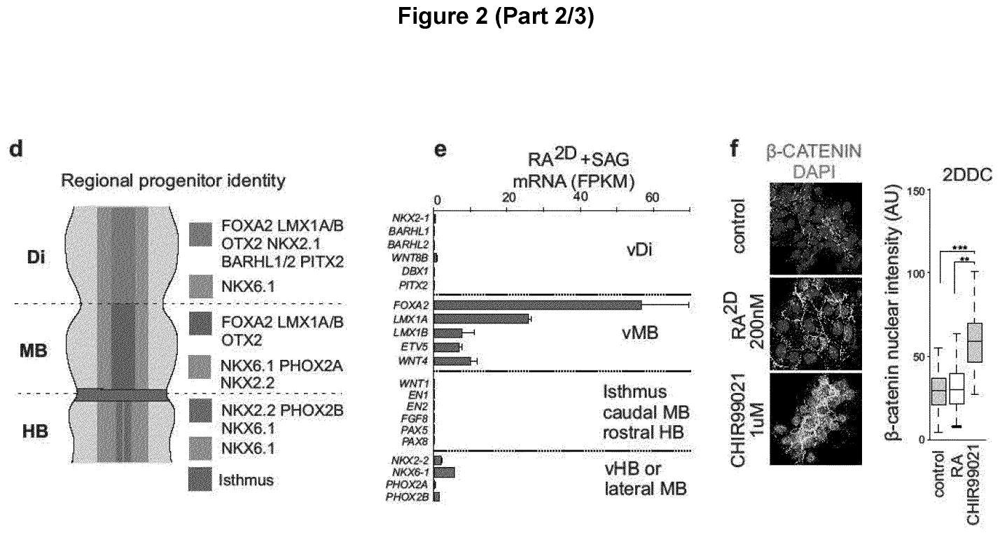

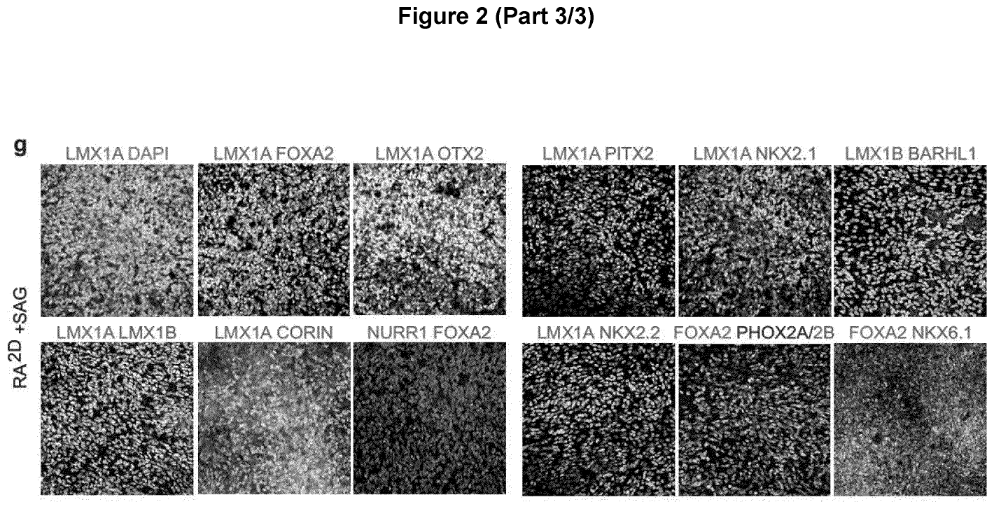

A LMX1A+/LMX1B+/FOXA2+/OTX2+ identity of neural stem cells was long considered as a molecular hallmark specific for vMB progenitors generating mDA neurons, but it was later shown that this identity is also shared by ventral progenitors in the caudal diencephalon giving rise to subthalamic nucleus neurons (STNs) (FIG. 2d). BARHL1, BARHL2, PITX2 and NKX2.1 are selectively expressed by the STN-lineage and thus can be used to distinguish between diencephalic STN-progenitors and ventral midbrain dopaminergic progenitor cells.

It will be appreciated that the ventral midbrain dopaminergic progenitor cells will not express markers indicative of FB identity, including FOXG1, EMX2, PAX6, SIX3, SIX6.

It will be appreciated that the ventral midbrain dopaminergic progenitor cells will not express markers indicative of HB identity, including NKX2.2, PHOX2B, HOXA2, HOXB4.

In a particular embodiment, the cell population comprises at least about 50%, at least about 60%, at least about 70%, or at least about 80% ventral midbrain dopaminergic progenitor cells.

In an embodiment, at least 60%, 65%, 70%, 75%, 80%, 85%, 90% or 95% of the cells are ventral midbrain dopaminergic progenitor cells positive for FoxA2 and/or Lmx1. In some embodiments, the cell population comprises at least 80%, 81%, 82%, 83%, 84%, 85%, 86%, 87%, 88%, 89%, 90%, 91%, 92%, 93%, 94%, 95%, 96%, 97%, 98%, 99% or more ventral midbrain dopaminergic progenitor cells (e.g., about 90%-98% or 95%-99% ventral midbrain dopaminergic progenitor cells) positive for FoxA2 and/or Lmx1.

In other words, the cell population is at least 60%, 65%, 70%, 75%, 80%, 85%, 90% or 95% positive for FoxA2 and/or Lmx1. This can be quantified using methods known in the art such as by counting the proportion of positive cells in a cell population following immunocytochemistry.

In a particular embodiment, the cell population comprises at least about 50%, at least about 60%, at least about 70%, or at least about 80% ventral midbrain dopaminergic progenitor cells at least after 7 days, such as about 9-16 days, such as about 14 days, after first contacting said cell population with the at least one activator of Retinoic Acid (RA) signalling.

In other words, the cell population comprises at least about 50%, at least about 60%, at least about 70%, or at least about 80% ventral midbrain dopaminergic progenitor cells at least after 7 DDC, such as about 9-16 DDC, such as about 14 DDC.

In a particular embodiment, the cell population comprises at least about 50%, at least about 60%, at least about 65%, at least about 70%, at least about 75%, or at least about 80% ventral midbrain dopaminergic progenitor cells 7-16 days, such as about 7 days, 8 days, 9 days, 10 days, 11 days, 12 days, 13 days, 14 days, or about 15 days after first contacting said cell population with the at least one activator of Retinoic Acid (RA) signalling. In an embodiment, the cell population comprises at least about 80% ventral midbrain dopaminergic progenitor cells about 14 days after first contacting said cell population with the at least one activator of Retinoic Acid (RA) signalling.

As described herein, ventral midbrain dopaminergic progenitor cells were derived from hPSCs within 7 days following initial exposure to RA (i.e. at 7 DDC, see definition below). These 7 DDC cells co-express FOXA2 and LMX1A. As can be seen in the accompanying Examples, within 14 days (i.e. by 14 DDC) about 80% of the cell population derived from hPSCs are ventral midbrain dopaminergic progenitor cells co-expressing FOXA2, LMX1A, LMX1B and OTX2 as well as the vMB marker CORIN (FIG. 2g) as determined by immunocytochemistry.

In a preferred embodiment, a population comprising ventral midbrain dopaminergic progenitor cells is obtainable within about 7-16 DDC, such as about within 8 DDC, such as about within 9 DDC, such as about within 10 DDC, such as about within 11 DDC, such as about within 12 DDC, such as about within 13 DDC, such as about within 14 DDC, such as within about 15 DDC.

Thus, the vast majority of hPSC-derived NSCs exposed to a timed RA pulse and small molecule activators of sonic hedgehog signalling (SHH) express LMX1A+/LMX1B+/FOXA2+/OTX2+ and have ventral midbrain (vMB) identity, with little contamination of cells expressing neighbouring diencephalic-, HB- or lateral MB-regional identities.

In a particular embodiment, the method is an in vitro method.

By “in vitro” we include an environment outside of the body. In vitro environments include but are not limited to, test tubes and cell cultures.

In a particular embodiment, the plurality of stem cell is selected from the group comprising: pluripotent stem cells; multipotent stem cells; non-embryonic stem cells such as adult stem cells (ASCs); and wherein the plurality of stem cells are derived from human, optionally wherein the human is a patient with a symptom of a neurological disorder; rodent; or primate.

The plurality of stem cells used to produce ventral midbrain dopaminergic progenitors can be obtained from a variety of sources including embryonic and non-embryonic sources, for example, hESCs and non-embryonic hiPSCs, somatic stem cells, disease stem cells, i.e. isolated pluripotent cells and engineered derived stem cells isolated from Parkinson disease patients, cancer stem cells, human or mammalian pluripotent cells, etc.

In a preferred embodiment, the plurality of stem cells are pluripotent stem cells. In an embodiment the plurality of stem cells are pluripotent stem cells derived from a human, primate, pig, dog or rodent. In a further preferred embodiment, the plurality of stem cells are human pluripotent stem cells.

By “pluripotent stem cell” we include a cell capable of giving rise to cells of all three germinal layers, that is, endoderm, mesoderm and ectoderm. Although in theory a pluripotent stem cell can differentiate into any cell of the body, the experimental determination of pluripotency is typically based on differentiation of a pluripotent cell into several cell types of each germinal layer. A pluripotent stem cell may be an embryonic stem (ES) cell derived from the inner cell mass of a blastocyst. In other embodiments, the pluripotent stem cell may be an induced pluripotent stem (iPS) cell obtained by inducing dedifferentiation of adult somatic cells through a method known in the art as cell reprogramming (Takahashi K., Yamanaka S. Cell. 2006; 126(4):663-676). In certain embodiments, the pluripotent stem cell is an embryonic stem cell derived by somatic cell nuclear transfer (NT-ESC).

A multipotent stem cell is a somatic stem cell which is capable of differentiating into all cell types of a given organ or tissue and to only cells of that organ or tissue. An Examples of a multipotent stem cells is a neural stem cell.

In a particular embodiment, the plurality of stem cells is selected from the group comprising: mouse pluripotent stem cells, mouse ESCs, mouse iPS cells, mouse neural stem cells, chemically induced stem cells, primate pluripotent stem cells, primate ESCs, primate iPS cells, primate neural stem cells, chemically induced primate stem cells, pig ESCs, pig iPS cells, pig neural stem cells, chemically induced pig stem cells, dog pluripotent stem cells, dog ESCs, dog iPS cells, dog neural stem cells, chemically induced dog stem cells, rat pluripotent stem cells, rat ESCs, rat iPS cells, rat neural stem cells, chemically induced rat stem cells, human pluripotent stem cells, human adult stem cells, human ESCs, human iPS cells, chemically induced human stem cells, NT-ESC, human amniotic stem cells, umbilical cord blood stem cells derived human ESCs, human neural stem cells, long-term neural stem cells derived from human ESCs, long-term neural stem cells derived from human iPS cells; and long-term neural stem cells derived from NT-ESCs, optionally wherein the human is a patient with a symptom of Parkinson's disease (PD).

Embryonic stem (ES) cells are pluripotent cells derived from the inner cell mass of a blastocyst. Methods for obtaining human ES cells and for the isolation of rhesus monkey and common marmoset ES cells are also known (Thomson et al, 1995 and Thomson, and Marshall, 1998). In one particular embodiment, said human ESCs have previously been derived by others without the destruction of embryo. For example, such cells may have been derived by extraction of a cell from an eight-cell blastocyst (Chung Y et al., (2008) Cell Stem Cell, February 7; 2(2):1 13-7). The skilled person is aware of other methods for derivation of ESCs without the destruction of embryos.

Another source of ES cells are established ES cell lines. Various mouse cell lines and human ES cell lines are known and conditions for their growth and propagation have been defined, for example, cells in a human W A-09 cell line. As used herein, by the term “ES cell line” we include the meaning of a clonal population of embryonic stem cells that express properties of pluripotency and that can be cultured under in vitro conditions that allow proliferation and propagation of cells without differentiation for up to days, months to years. Different ES cell lines are established independently from each other and are different by genotype and to certain extent also by phenotype, such as different responsiveness to developmental signalling molecules. It will be appreciated that all such ES cell lines may be used in the invention.

Induced pluripotent stem (iPS) cells are cells which have the characteristics of ES cells but are obtained by the reprogramming of differentiated somatic cells. iPSCs are able to self-renew in vitro and differentiate into cells of all three germ layers that is, endoderm, mesoderm and ectoderm. iPS cells have been obtained by various methods known in the art, and unlike an ES cell an iPSC is formed artificially by the introduction of certain embryonic genes (such as an OCT4, SOX2, and KLF4 transgene). Mouse iPSCs were reported in 2006 (Takahashi and Yamanaka), and human iPSCs were reported in late 2007 (Takahashi et al. and Yu et al.).

The iPS cell can be a mammalian cell, for example a mouse, human, rat, bovine, ovine, horse, hamster, dog, guinea pig, or non-human primate cell. For example, reprogramming of somatic cells provides an opportunity to generate patient- or disease-specific pluripotent stem cells. iPS cells are indistinguishable from ES cells in morphology, proliferation, gene expression, and teratoma formation. Human iPS cells are also expandable and indistinguishable from human embryonic stem (ES) cells in morphology and proliferation.

Mesenchymal cells can be useful for creating iPS cells and may be obtained from any suitable source and may be any specific mesenchymal cell type. For example, if the ultimate goal is to generate therapeutic cells for transplantation into a patient, mesenchymal cells from that patient are desirably used to generate the iPS cells. Suitable mesenchymal cell types include fibroblasts (such as skin fibroblasts), hematopoietic cells, hepatocytes, smooth muscle cells, and endothelial cells. In suitable embodiments, the iPS cells used in the present methods are derived from a PD patient.

An embryonic stem cell derived by somatic cell nuclear transfer (NT-ESC) is a pluripotent stem cell prepared by means of somatic cell nuclear transfer, in which a donor nucleus is transferred into a spindle-free oocyte for example as described by Tachibana et al., (2013) Cell; 153(6):1 228-38.

As used herein, the term “somatic (adult) stem cell” refers to a relatively rare undifferentiated cell found in many organs and differentiated tissues with a limited capacity for both self-renewal and differentiation. An example includes a hematopoietic stem cell that gives rise to all red and white blood cells and platelets.

As used herein, the term “umbilical cord blood stem cells” refer to stem cells collected from an umbilical cord at birth that have the capability to at least produce all of the blood cells in the body (hematopoietic).

Methods for cell culturing and differentiating pluripotent stem cells may be carried out with reference to standard literature in the field. Suitable techniques are described by Lemke, Kristen A., Alireza Aghayee, and Randolph S. Ashton. “Deriving, regenerating, and engineering CNS tissues using human pluripotent stem cells.” Current opinion in biotechnology 47 (2017): 36-42.

The skilled person is aware of cell culture media that are suitable for neural stem cell growth, such as but not limited to any modifications of basic media such as DMEM, F 12, RPMI 1640 and MEM. The skilled person is aware of that basic media can be modified for many different purposes. Non-limiting examples of suitable media include example Neurobasal™ medium and NSC™ from Life Technologies, PNGM™ from Lonza, Neural Stem Cell basal medium from Millipore, Knockout® Serum Replacement (“KSR”) medium from ThermoFisher Scientific, Essential 8®/Essential 6® (“E8/E6”) medium from ThermoFisher Scientific, and Stemdiff™ from StemCell Technologies.

It will be appreciated that different cell culture mediums are illustrated, which are modified by the addition of differentiation factors and/or patterning factors to arrive at multiple different cell culture mediums.

In one particular embodiment, the cell culture medium comprises DMEM/F12 and Neurobasal medium. In a preferred embodiment the cell culture medium is a cell culture medium comprising a Neurobasal medium (ThermoFisher Catalog number: 21103049) supplemented with N2 (ThermoFisher; Catalog number: 17502048), and B27 (ThermoFisher) (containing vitamin A) (see Ying, Qi-Long, et al. “Conversion of embryonic stem cells into neuroectodermal precursors in adherent monoculture.” Nature biotechnology 21.2 (2003): 183-186).

In one embodiment, the cell culture medium is a cell culture medium supplemented with one or more soluble factors selected from the group comprising N2, B27 (containing vitamin A), β-mercaptoethanol, and L-glutamine (such as Glutamax™). In one particular embodiment, said cell culture medium is supplemented with at least N2 and B27 (containing vitamin A). In an embodiment, this medium, termed “N2B27 medium” preferably comprises DMEM/F12: Neurobasal (1:1), 0.5×N2 and 0.5×B27 (containing vitamin A), 1×nonessential amino acids, 1% GlutaMAX, and 55 μM β-mercaptoethanol.

In some embodiments, the culture conditions for differentiation may comprise dissociating the cells into a substantially single cell culture. As can be seen in the accompanying Examples, the cells were dissociated prior to plating and at 9 DDC and 23 DDC. The dissociation encompasses the use of any method known now or later developed that is capable of dissociating cells into smaller groups or into a single cell suspension. In an exemplary embodiment, the cells may be dissociated by a protease treatment, or a mechanical treatment like pipetting, or using a Stem Cell Passaging Tool (Thermo Fisher Scientific) as described in the accompanying Examples. For example, the protease may be Accutase (Thermo Fisher Scientific), collagenase, trypsin-EDTA, dispase, or a combination thereof. Alternatively, a chelating agent may be used to dissociate the cells, such as sodium citrate, EGTA, EDTA or a combination thereof. An essentially single cell culture may be a cell culture wherein the cells desired to be grown are dissociated from one another, such that the majority of the cells are single cells, or at most two cells that remain associated (doublets).

In certain aspects, single cell culture may be in the presence of a small molecule effective for increasing cloning efficiency and cell survival following dissociation, such as a ROCK inhibitor or myosin II inhibitor, as described in the accompanying Examples.

In certain embodiments, the cells can be cultured while attached to a solid or semi-solid substrate (adherent or monolayer culture) as described in the accompanying Examples. Various matrix components are known in the art an may be used in culturing, maintaining, or differentiating human pluripotent stem cells. For example, substrates for cell adhesion include collagen, gelatin, poly-L-lysine, poly-D-lysine, poly-L-ornithine, laminin, vitronectin, and fibronectin and mixtures thereof, such as Matrigel™ or Geltrex, and lysed cell membrane preparations, which may be used to coat a culturing surface as a means of providing a solid support for pluripotent cell growth, as described in the accompanying Examples.

Cells can also be grown floating in the culture medium (suspension culture).

In certain embodiments, non-static culture could be used for culturing and differentiation of pluripotent stem cells. The non-static culture can be any culture with cells kept at a controlled moving speed, by using, for example, shaking, rotating, or stirring platforms or culture vessels, particularly large-volume rotating bioreactors. Agitation may improve circulation of nutrients and cell waste products and also be used to control cell aggregation by providing a more uniform environment. For example, rotary speed may be set to at least or at most about 25, 30, 35, 40, 45, 50, 75, 100 rpm, or any range derivable therein.

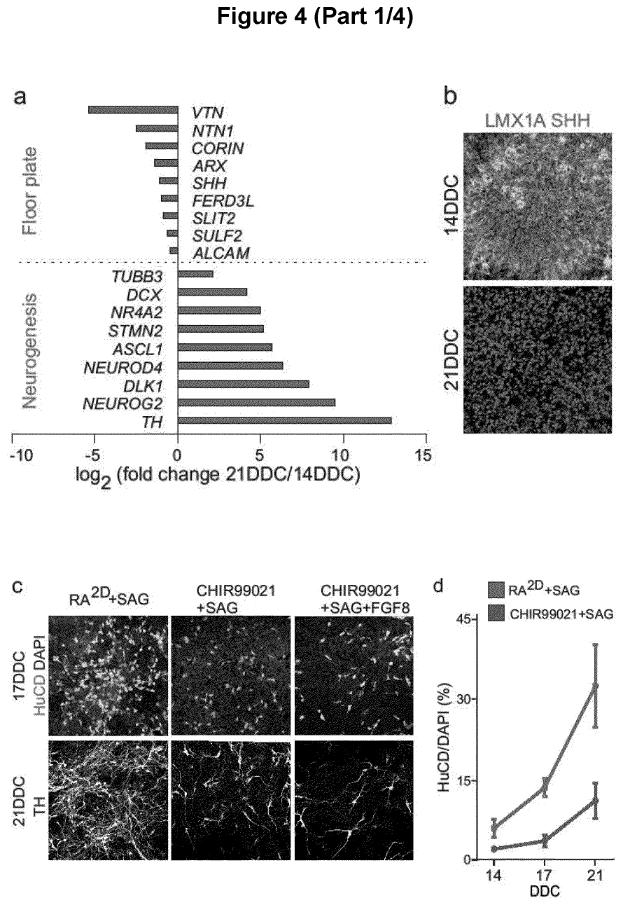

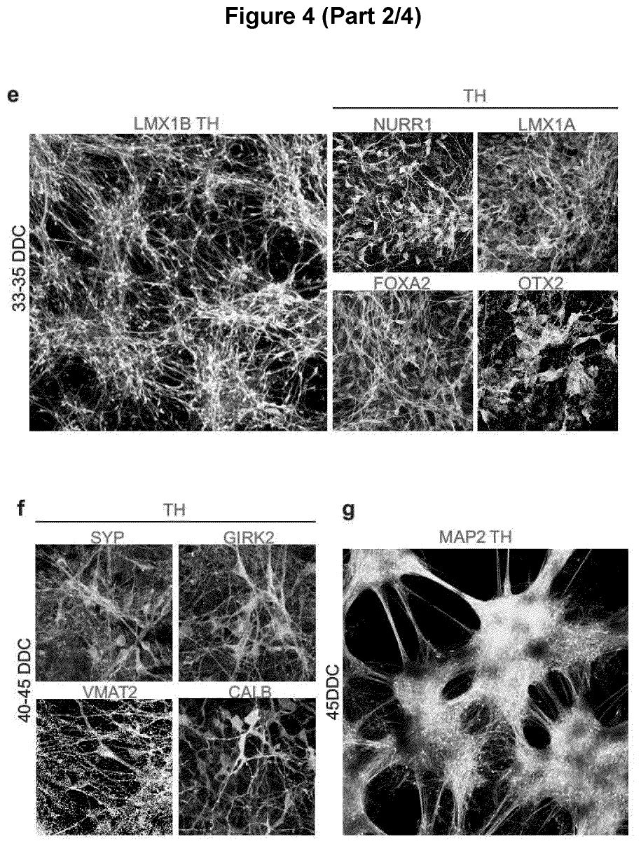

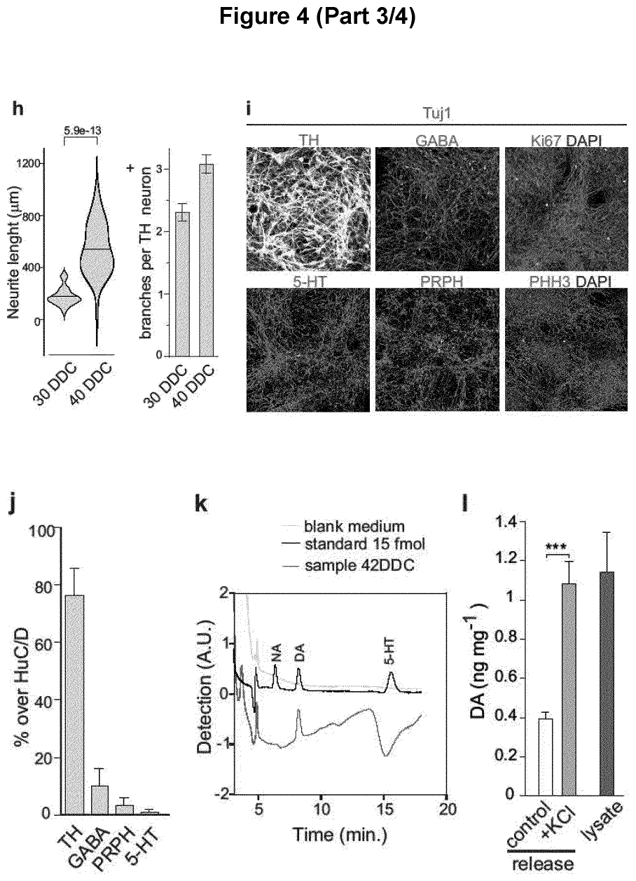

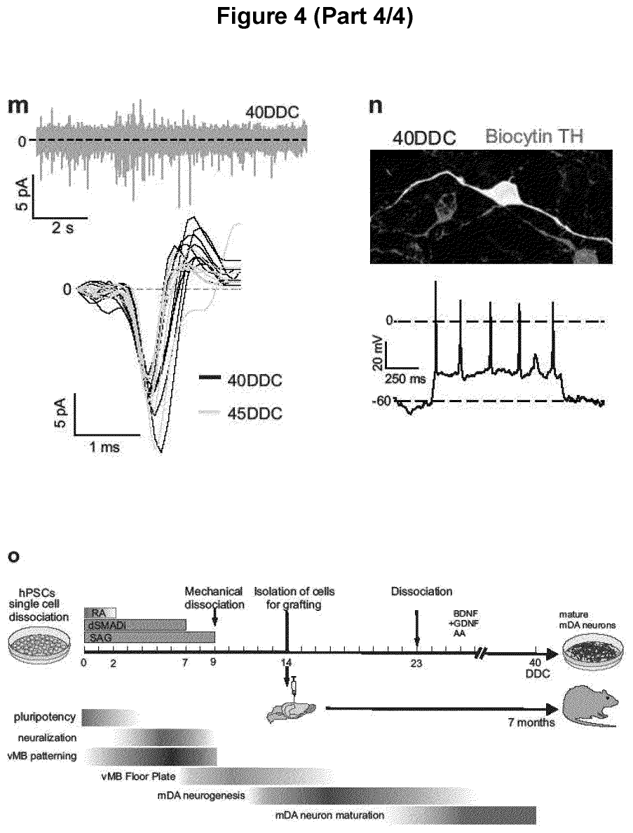

It will be appreciated that culturing the stem cells under conditions sufficient to cause differentiation of the stem cells into a cell population comprising ventral midbrain dopaminergic progenitor cells typically comprises contacting the stem cells with one or more factors, added at various timepoints for various durations. Conveniently, these timepoints and durations are described by reference to “days in differentiation condition (DDC)” nomenclature. For example, 1 DDC refers to the fact that these cells have been in the differentiation culture for 1 day, and 2 DDC refers to the fact that these cells have been in the differentiation culture for 2 days, and so on. In this way, DDC can be used as a reference for a timepoint in the differentiation culture, with 1 DDC corresponding to day 1, 2 DDC corresponding to day 2 and so on. Similarly, the duration and timepoint of exposure can be described by reference to DDC. For example, the cells may be exposed to a particular factor for 2 days between 3 DDC and 5 DDC. As described in the accompanying Examples, the differentiation protocol begins on day 0 (i.e. 0 DDC) when the cells are plated. After 14 days (14 DDC), the cells can be prepared for transplantation (see FIG. 4 o).

In an embodiment, culturing the stem cells under conditions sufficient to cause differentiation of the stem cells into a cell population comprising ventral midbrain dopaminergic progenitor cells comprises contacting the stem cells with at least one activator of Hedgehog (Hh) signalling.

Hedgehog (HH or Hh) signalling is known to play a key role in regulating vertebrate organogenesis, such as in the growth of digits on limbs and organization of the brain. The vertebrate hedgehog protein family consists of Sonic Hedgehog (SHH), Indian Hedgehog (IHH) and Desert Hedgehog (DHH), which share many functional characteristics and signal through a common pathway. For example, during the development of the CNS, SHH acts as a morphogen, a molecule that diffuses to form a concentration gradient, and as such has different effects on the cells of the developing nervous system depending on its concentration. Briefly, Hh signals by interacting with the Hh receptor complex comprising two components; Patched (Ptc) and Smoothened (Smo) that transduce the Hh signal into the cell. Ptc is considered to repress Hh signalling by binding to Smo in the cell membrane. In the presence of Hh ligand, this repression is relieved and Smo is able to signal. In vertebrates, the zinc finger proteins Gli1, Gli2 and Gli3 are downstream mediators of Hh signalling and are involved in controlling the transcriptional response of target genes in a Hh dependent manner.

The skilled person will appreciate that the term “activator of Hh signalling” includes factors that potentiate or substitute for Hh signalling, or derivative or agonists thereof. Such factors may be involved in signalling downstream of Hh or be small molecule agonists.

In an embodiment, the at least one activator of the Hedgehog (Hh) signalling is selected from the group comprising: Sonic Hedgehog (SHH), Indian hedgehog (IHH), Desert hedgehog (DHH), purmorphamine, Smoothened agonists (SAGs) such as SAG 1.3 (Hh-1.3), Hh-1.2, Hh-1.4, Hh-1.5, and combinations thereof.

In a preferred embodiment, the activator of Hh signalling is SAG 1.3. Such activators are commercially available from, for example, Santa Cruz Biotechnology.

In certain embodiments, the at least one activator of Hh signalling is contacted to the cells for at least about 4, 5, 6, 7, 8, 9, or 10 or more days, for example, between about 4 and 10 days, or between about 5 and 9 days, or between about 6 and 9 days. In certain embodiments, the at least one activator of Hh signalling is contacted to the cells for up to about 4, 5, 6, 7, 8, 9, or 10 or more days. In certain embodiments, the at least one activator of Hh signalling is contacted to the cells for about 8-9 days. In certain embodiments, the at least one activator of Hh signalling is contacted to the cells for about 9 days from 0 to 9 DDC.

As can be seen from the accompanying Examples and FIG. 8c (Supplementary FIG. 2c)), initial exposure of the stem cells to the at least one activator of Hedgehog (Hh) signalling at day 0 DDC, or day 1 DDC resulted in effective induction of a vMB identity to NSCs. Accordingly, in certain embodiments, the at least one activator of Hh signalling is contacted to the cells for about 8 days from day 1 DDC to day 9 DDC. In another embodiment, the at least one activator of Hh signalling is contacted to the cells for about 9 days from day 0 DDC to day 9 DDC

In certain embodiments, the at least one activator of Hh signalling is added every day or every other day to a cell culture medium comprising the stem cells from day 0 to day 9. In certain embodiments, the at least one activator of Hh signalling is added every day or every other day to a cell culture medium comprising the stem cells from day 1 to day 9. In certain embodiments, the at least one activator of Hh signalling is added on days 0, 2, 4, 6, 8 DDC. In certain embodiments, the at least one activator of Hh signalling is added on days 1, 3, 5, 7, 9 DDC.

In certain embodiments, the at least one activator of Hh signalling is contacted to the cells at a concentration of between about 50 and 1000 nM, or between about 100 and 950 nM, or between about 150 and 900 nM, or between about 200 and 850 nM, or between about 250 and 800 nM, or between about 300 and 750 nM, or between about 350 and 700 nM, or between about 400 and 650 nM, or between about 450 and 600 nM, or between about 500 and 550 nM, and values in between. In certain embodiments, the one or more activator of Hh signalling is contacted to the cells at a concentration of about 400, 450, 500, 550, or 600 nM. In certain embodiments, the at least one activator of Hh signalling is contacted to the cells at a concentration of about 300 nM.

As can be seen in the accompanying Examples, titration data presented in FIG. 8d (Supplementary FIG. 2D) demonstrates that effective induction of a vMB identity to stem cells was possible when the at least one activator of Hh signalling was used at a concentration 50 nM-1000 nM.

In a specific, non-limiting embodiment, the cells are contacted with at least one activator of Hh signalling, for example, SAG 1.3 at a concentration of about 300 nM; for about 9 days (i.e. from 0 to 9 DDC).

In an alternative specific, non-limiting embodiment, the cells are contacted with at least one activator of Hh signalling, for example, SAG 1.3 at a concentration of about 300 nM; for about 8 days (i.e. from 1 to 9 DDC).

In a preferred embodiment, culturing the stem cells under conditions sufficient to cause differentiation of the stem cells into a cell population comprising ventral midbrain dopaminergic progenitor cells comprises contacting the stem cells with at least one inhibitor of TGFβ/Activin-Nodal signalling and at least one inhibitor of bone morphogenetic protein (BMP) signalling.

As used herein, an “inhibitor of TGFβ/Activin-Nodal signalling” may be referred to simply as a “TGFβ/Activin-Nodal inhibitor.” Similarly, an “inhibitor of BMP signalling” may be referred to herein simply as a “BMP inhibitor.”

By “inhibitor” we include any compound or molecule (e.g., small molecule, peptide, peptidomimetic, natural compound, siRNA, anti-sense nucleic acid, aptamer, or antibody) that interferes with (e.g., reduces, decreases, suppresses, eliminates, or blocks) the signalling function of the molecule or pathway. An inhibitor can be any compound or molecule that changes any activity of a particular protein signalling molecule, any molecule involved with the particular signalling molecule.

For example, the inhibitor of TGFβ/Activin-Nodal signalling may act via directly contacting SMAD signalling, contacting SMAD mRNA, causing conformational changes of SMAD, decreasing SMAD protein levels, or interfering with SMAD interactions with signalling partners (e.g., including those described herein), and affecting the expression of SMAD target genes (e.g. those described herein). An inhibitor may diminish or decrease the pathway to be inhibited by 10%, 20%, 30%, 40%, 50%, 60%, 70%, 80%, 90%, 100% or more when compared to the activity of the pathway without or before the addition of the inhibitor.

Inhibitors of TGFβ/Activin-Nodal signalling also include molecules that indirectly regulate biological activity, for example, SMAD biological activity, by intercepting upstream signalling molecules (e.g., within the extracellular domain, examples of a signalling molecule and an effect include: Noggin which sequesters bone morphogenic proteins, inhibiting activation of ALK receptors 1, 2, 3, and 6, thus preventing downstream SMAD activation. Likewise, Chordin, Cerberus, Follistatin, similarly sequester extracellular activators of SMAD signalling. Bambi, a transmembrane protein, also acts as a pseudo-receptor to sequester extracellular TGFβ signalling molecules). Antibodies that block upstream or downstream proteins may also be used to neutralize extracellular activators of protein signalling. Inhibitors are described in terms of competitive inhibition (binds to the active site in a manner as to exclude or reduce the binding of another known binding compound) and allosteric inhibition (binds to a protein in a manner to change the protein conformation in a manner which interferes with binding of a compound to that protein's active site) in addition to inhibition induced by binding to and affecting a molecule upstream from the named signalling molecule that in turn causes inhibition of the named molecule. An inhibitor can be a “direct inhibitor” that inhibits a signalling target or a signalling target pathway by actually contacting the signalling target.

In certain embodiments, the presently invention provides methods for differentiating stem cells into ventral midbrain dopaminergic progenitors comprising contacting a population or plurality of human stem cells with one or more inhibitor of TGFβ/Activin-Nodal signalling (i.e., a first SMAD inhibitor) and one or more inhibitor of BMP signalling (i.e., a second SMAD inhibitor).

The inhibition of TGFβ/Activin-Nodal signalling and BMP signalling is termed “dual inhibition of SMAD signalling” or “dual SMAD inhibition” or “dSMADi”. Dual SMAD inhibition has been used previously as a rapid and highly effective method for inducing one type of neural lineage cells from hPSCs (Chambers, et al., Nat Biotechnol 27, (2009)).

The mammalian SMAD protein family is a family of eight members that serve as intracellular signalling mediators of the TGFβ superfamily. Smad2 and Smad3 mediate TGFβ and activin/inhibin signalling, while BMP signalling is mediated by Smad1, Smad5 and Smad8.

It is known in the art that TGF signalling is involved in embryogenesis, cell differentiation and apoptosis as well as in other functions. TGF super family ligands, for example, TGFB1, TGFB2, TGFB3, ACTIVIN A, ACTIVIN B, ACTIVIN AB and/or NODAL, bind to a heterotetrametric receptor complex consisting of two type I receptor kinases (also termed ALK5), including, for example, TGFBR2, ACVR2A, and/or ACVR2B, and two type II receptor kinases, including, for example, TGFBR1, ACVR1B, and/or ACVR1C. This binding induces phosphorylation and activation of a heteromeric complex consisting of an R-SMAD, including, for example, SMAD2, and/or SMAD3, and a Co-SMAD, including, for example, SMAD4. RSMAD/CoSMAD complexes accumulate in the nucleus where they act as transcription factors and participate in the regulation of target gene expression.

The skilled person will appreciate that the term “inhibitor of TGFβ/Activin-Nodal signalling” refers to inhibitors of any one of the molecules that form part of this signalling pathway. For example, the inhibitor can be an antagonist of the ACVR2A and/or ACVR1B (ALK4) receptor or an antagonist of the TGFβ type II receptor kinases and/or ALK5 receptor. Such inhibitors of the TGFβ/Activin-Nodal signalling pathway are known in the art and are commercially available.

In a preferred embodiment, the at least one inhibitor of TGFβ/Activin-Nodal signalling is selected from the group comprising SB431542 and SB505124.

The invention contemplates that the TGFβ/Activin-Nodal signalling inhibitor is an inhibitor of the TGFβ type I receptor. In an embodiment, the TGFβ/Activin-Nodal signalling inhibitor inhibits ALK5 and also the activin type I receptor ALK4 and/or the nodal type I receptor ALK7, which are very highly related to ALK5 in their kinase domains.

Exemplary, non-limiting examples of an TGFβ/Activin-Nodal signalling inhibitor include SB431542 (CAS No.: 301836-41-9), SB-505124 (CAS No.: 694433-59-5), A-83-01, GW6604, IN-I 130, Ki26894, LY2157299, LY364947 (HTS-466284), LY550410, LY5 73636, LY580276, NPC-30345, SD-093, Sml6, SM305, SX-007, Antp-Sm2A, GW788388, LY2109761, and R 268712, D 4476, ITD 1, and RepSox. Non-limiting examples of inhibitors of TGFβ/Activin-Nodal signalling are also disclosed in Chambers, et al., Nat Biotechnol 27, (2009), and these inhibitors are incorporated by reference. In certain embodiments, the at least one inhibitor of TGFβ/Activin-Nodal signalling is SB431542 and derivatives thereof. For example, SB431542 can be obtained from Miltenyi Biotech.

In certain embodiments, the at least one inhibitor of TGFβ/Activin-Nodal signalling is contacted to the cells for at least about 2, 3, 4, 5, 6, 7, 8, 9, or 10 or more days. In certain embodiments, the at least one inhibitor of TGFβ/Activin-Nodal signalling is contacted to the cells for at least about 3 and 9 days. In certain embodiments, the at least one inhibitor of TGFβ/Activin-Nodal signalling is contacted to the cells for at least about 4 and 8 days. In certain embodiments, the at least one inhibitor of TGFβ/Activin-Nodal signalling is contacted to the cells for at least about 5 and 7 days. In certain embodiments, the at least one inhibitor of TGFβ/Activin-Nodal signalling is contacted to the cells for about 7 days from day 0 DDC to day 7 DDC.

In certain embodiments, the at least one inhibitor of TGFβ/Activin-Nodal signalling is added every day or every other day to a cell culture medium comprising the stem cells from day 0 to day 10. In certain embodiments, the at least one inhibitor of TGFβ/Activin-Nodal signalling is added on days 0, 2, 4, and 6. In an embodiment, the medium is changed every other day, i.e. on alternate days, and fresh inhibitor is added.

In certain embodiments, the at least one inhibitor of TGFβ/Activin-Nodal signalling is contacted to the cells at a concentration of between about 1 and 50 μM, or between about 1 and 20 μM, or between about 2 and 15 μM, or between about 3 and 10 μM, or between about 5 and 10 μM, and values in between. In certain embodiments, the at least one inhibitor of Fβ/Activin-Nodal signalling is contacted to the cells at a concentration of about 5, 6, 7, 8, 9, or 10 μM. In certain embodiments, the at least one inhibitor of Fβ/Activin-Nodal signalling is contacted to the cells at a concentration of about 5 μM.

In a specific, non-limiting embodiment, the cells are contacted with at least one inhibitor of TGFβ/Activin-Nodal signalling (i.e. a first SMAD inhibitor), for example, SB431542 at a concentration of about 5-10 μM for about 7 days (i.e. from day 0 DDC to day 7 DDC).

The BMP signalling pathway is known in the art (Jiwang Zhanga, Linheng Lia (Developmental Biology Volume 284, Issue 1, (2005), Pages 1-11).

In short, BMP functions through receptor-mediated intracellular signalling and subsequently influences target gene transcription. Two types of receptors are required in this process, which are referred to as type I and type II. While there is only one type II BMP receptor (BmprII), there are three type I receptors: Alk2, Alk3 (Bmpr1 a), and Alk6 (Bmpr1 b). BMP signal transduction can take place over at least two signalling pathways. The canonical BMP pathway is mediated by receptor I mediated phosphorylation of Smad1, Smad5, or Smad8 (R-Smad). Two phosphorylated R-Smads form a heterotrimeric complex co-aggregate with a common Smad4 (co-Smad). The Smad heterotrimeric complex can translocate into the nucleus and can cooperate with other transcription factors to modulate target gene expression. A parallel pathway for the BMP signal is mediated by TGFβ1 activated tyrosine kinase 1 (TAK1, a MAPKKK) and through mitogen activated protein kinase (MAPK), which also involves cross-talk between the BMP and Wnt pathways.

In an embodiment the BMP signalling inhibitor is a canonical BMP signalling inhibitor. Exemplary non-limiting examples of BMP signalling inhibitors include DMH1 (CAS 1206711-16-1); DMH2; LDN-193189 (CAS No.: 1062368-24-4); LDN-214117; chordin; gremlin; ventropin; follistatin; noggin; K02288; and Dorsomorphin (CAS No.: 866405-64-3). DMH-1 can for example be obtained from Santa Cruz Biotech.

In a preferred embodiment, at least one inhibitor of BMP signalling is selected from the group comprising: DMH-1; LDN-193189; and Noggin.

In certain embodiments, the at least one inhibitor of BMP signalling is contacted to the cells for at least about 2, 3, 4, 5, 6, 7, 8, 9, or 10 or more days. In certain embodiments, the at least one inhibitor of BMP signalling is contacted to the cells for at least about 3 and 9 days. In certain embodiments, the at least one inhibitor of BMP signalling is contacted to the cells for at least about 4 and 8 days. In certain embodiments, the at least one inhibitor of BMP signalling is contacted to the cells for at least about 5 and 7 days. In certain embodiments, the at least one inhibitor of BMP signalling is contacted to the cells for about 7 days from day 0 DDC to day 7 DDC.

In certain embodiments, the at least one inhibitor of BMP signalling is added every day or every other day to a cell culture medium comprising the stem cells from day 0 to day 10. In certain embodiments, the at least one inhibitor of BMP signalling is added on days 0, 2, 4, and 6. In an embodiment, the medium is changed every other day, i.e. on alternate days, and fresh inhibitor is added.

In certain embodiments, the at least one inhibitor of BMP signalling is contacted to the cells at a concentration of between about 0.01 μM and about 50 μM, or between about 1 and 25 μM, or between about 1 and 15 μM, or between about 1 and 10 μM, or between about 1 and 5 μM, or between about 2 and 4 μM and values in between. In certain embodiments, the at least one inhibitor of BMP signalling is contacted to the cells at a concentration of about 2.5 μM.

In a specific, non-limiting embodiment, the cells are contacted with at least one inhibitor of BMP signalling (i.e. a second SMAD inhibitor), for example, DMH1 at a concentration of about 250 nM for about 7 days (i.e. from day 0 DDC to day 7 DDC).

Dual inhibition of SMAD can be achieved with a variety of compounds such as those described above including Noggin, SB431542, LDN-193189, DMH-1, Dorsomorphin, or other molecules that block TGFβ, BMP, and Activin/Nodal signalling. A preferred embodiment comprises the use of SB431542 and DMH-1 at a concentration of 0.1 μM-250 μM, or more preferable 1-25 μM, or most preferable 5 μM of SB431542 and 2.5 μM of DMH-1.

In a specific, non-limiting embodiment, the cells are contacted with at least one inhibitor of TGFβ/Activin-Nodal signalling (i.e. a first SMAD inhibitor), for example, SB431542 at a concentration of about 5 μM for about 7 days (i.e. from day 0 DDC to day 7 DDC); at least one inhibitor of BMP signalling (i.e. a second SMAD inhibitor), for example, DMH1 at a concentration of about 250 nM for about 7 days (i.e. from day 0 DDC to day 7 DDC); and at least one activator of Hh signalling, for example, SAG 1.3 at a concentration of about 300 nM; for about 9 days (i.e. from day 0 DDC to day 9 DDC), or for about 8 days (i.e. from day 1 DDC to day 9 DDC).

In a preferred embodiment, the stem cells are contacted with the activator of retinoic acid (RA) signalling for about 1-4 days, optionally about 1-3 days.

By “1-4 days”, as used herein, we include that the activator of retinoic acid is contacted with the stem cells for a duration of about 24 to 96 hours. By “1-3 days”, as used herein, we include 24 to 72 hours. In a further embodiment, the stem cells are contacted with the activator of retinoic acid (RA) signalling for a duration of about 1.5-3 days, i.e. about 36 to 72 hours.

As shown in the accompanying Examples, when the activator of retinoic acid (RA) signalling is EC23, EC23 could differentiate pluripotent stem cells into ventral midbrain dopaminergic progenitor cells following contact with the stem cells for about 1 day (i.e. about 24 hours), (i.e. from 0 to 1 DDC, or from 1 to 2 DDC, or from 2 to 3 DDC, or from 3 to 4 DDC. When the activator of retinoic acid (RA) signalling is ATRA, ATRA could differentiate pluripotent stem cells into ventral midbrain dopaminergic progenitor cells following contact with the stem cells for about 2 days (i.e. about 48 hours) from 0 DDC to 2 DDC.

In a preferred embodiment, the at least one activator of retinoic acid (RA) signalling is not present at an effective amount after contacting the plurality of stem cells for about 1-4 days, optionally about 1-3 days.

In an embodiment, after the plurality of stem cells is contacted with the plurality of stem cells for about 1-4 days, optionally about 1-3 days, the stem cells are cultured in the absence of an effective amount of the at least one activator of retinoic acid (RA) signalling for at least the following 7 days.

As described in the accompanying Examples, the at least one activator of retinoic acid (RA) signalling is delivered as a “pulse” and so it is removed from the culture medium after the pulse.

In an embodiment, the stem cells are contacted with the at least one activator of Hedgehog (Hh) signalling, the at least one inhibitor of TGFβ/Activin-Nodal signalling, and the at least one inhibitor of bone morphogenetic protein (BMP) signalling simultaneously with the at least one activator of retinoic acid (RA) signalling.

As can be seen from the accompanying Examples and FIG. 4 o, exposure of stem cells to the at least one activator of Hedgehog (Hh) signalling (i.e. SAG) and the at least one inhibitor of TGFβ/Activin-Nodal signalling, and the at least one inhibitor of bone morphogenetic protein (BMP) signalling (i.e. dual SMAD inhibition) and the at least one activator of retinoic acid (RA) signalling resulted in effective induction of a vMB identity to NSCs.

In an alternative embodiment, the stem cells are contacted with the at least one activator of Hedgehog (Hh) signalling, the at least one inhibitor of TGFβ/Activin-Nodal signalling, and the at least one inhibitor of bone morphogenetic protein (BMP) signalling prior to being contacted with the at least one activator of retinoic acid (RA) signalling.

As can be seen from the accompanying Examples and FIG. 8b (Supplementary FIG. 2b), initial exposure of the stem cells to the at least one activator of retinoic acid (RA) signalling between day 0 and day 2 DDC for the following 48 hours resulted in effective induction of a vMB identity to NSCs. Accordingly, in an embodiment the at least one activator of retinoic acid (RA) signalling is contacted to the cells for about 2 days from 0 to 2 DDC; or for about 2 days from 1 to 3 DDC; or for about 2 days from 2 to 4 DDC. In other words, the RA-pulse is to be initiated between 0-2 DDC.

As can be seen from the accompanying Examples and FIG. 8c (Supplementary FIG. 2c), initial exposure of the stem cells to the at least one activator of Hedgehog (Hh) signalling at day 0 DDC, or day 1 DDC resulted in effective induction of a vMB identity to NSCs.

The inventors have surprisingly found that neither WNT agonists nor FGF is required in the RA-based specification of LMX1A/B+/FOXA2+/OTX2 ventral midbrain dopaminergic progenitor cells, or in the production of mature mDA neurons. As described in the accompanying Examples, (dSMADi) is deployed to promote a generic neural fate by preventing hPSCs from selecting alternative somatic or extraembryonic fate options; the at least one activator of retinoic acid (RA) signalling promotes a switch-like transition from pluripotency into an NSC-state and concomitantly imposes a MB-like identity to NSCs; and the at least one activator of Hedgehog (Hh) signalling is applied to ventralize cells and induce a LMX1A+/FOXA2+/OTX2+ vMB identity characteristic of mDA neuron progenitors. The inventors found that stem cells treated with RA but not WNT acquire a more rostral ventral midbrain identity characterised by the expression of LMX1A/B+/FOXA2+/OTX2+EN1−, rather than a caudal midbrain identity.

In an embodiment, the method comprises the sequential addition of an activator of WNT signalling after the initial contacting of a plurality of stem cells with the at least one activator of retinoic acid (RA) signalling to induce expression of ENGRAILED-1 (EN1), which is a marker of the caudal midbrain. This will give rise to caudalised ventral mesencephalon progenitor cells (LMX1A/B+/FOXA2+/OTX2+EN1+). Accordingly, it will be appreciated that Wnt is not used in the method of the invention to specify a ventral midbrain identity to stem cells.

As described in the accompanying Examples, the inventors surprisingly found that EN1 expression is upregulated in RA-induced midbrain progenitor cells treated with the Wnt signalling agonist CHIR99021. The inventors found that the optimal concentration of CHIR99031 for the generation of EN1+LMX1A+FOXA2+OTX+ progenitor cells is between 0.6 μM and 10 μM, such as between 2.5 μM and 10 μM. In an embodiment, CHIR99021 is used in the method of the invention at a concentration of 5 μM.

EN1 (ENGRAILED-1) is a homeobox gene that regulates development in the caudal midbrain and anterior hindbrain. Graded expression of EN1 depend on signalling by WNT1 expressed by the isthmic organized localized at the boundary between the MB and HB.

As shown in the accompanying Examples, the plurality of stem cells were contacted with the activator of wingless (WNT) signalling CHIR99021 at 4-9 DDC (FIG. 6). In a specific, non-limiting embodiment, the cells are contacted with an activator of WNT signalling for about 4-6 days, preferably for about 5 days. In a further embodiment, the cells are contacted with an activator of WNT signalling at 4-9 DDC.

In an embodiment, the plurality of stem cells are contacted with the activator of wingless (WNT) signalling after the stem cells have been contacted with the at least one activator of RA signalling. Preferably, the plurality of stem cells are contacted with the activator of wingless (WNT) signalling about 24 hours after the stem cells have been contacted with the at least one activator of RA signalling, such as about 36 hours after, such as about 48 hours after, such as about 60 hours after, such as about 72 hours after, such as about 84 hours after, such as about 96 hours after the stem cells have been contacted with the at least one activator of RA signalling.

Accordingly, it will be appreciated that the method may comprise sequentially contacting the plurality of stem cells with the at least one activator of retinoic acid (RA) signalling and an activator of wingless (WNT) signalling, and preferably the stem cells are contacted sequentially with the at least one activator of retinoic acid (RA) signalling before the activator of wingless (WNT) signalling.

In an embodiment, the method does not comprise contacting the plurality of stem cells with an activator of wingless (WNT) signalling simultaneously with the at least one activator of retinoic acid (RA) signalling.

By “WNT” or “wingless” in reference to a signalling pathway we include a signal pathway composed of Wnt family ligands and Wnt family receptors, such as Frizzled and Derailed/RYK receptors, mediated with or without βcatenin. For the purposes described herein, a preferred WNT signalling pathway includes mediation by β-catenin, i.e. canonical WNT signalling.

For example, the activator of WNT signalling can be a glycogen synthase kinase 3 (GSK3) inhibitor. Non-limiting examples of inhibitors GSK3 inhibitors include CHIR9902, NP031112, TWSI 19, SB216763, CHIR-98014, AZD2858, AZD1080, SB415286, LY2090314 and 1. In certain aspects, the activator of WNT signalling may be CHIR99021 available from Miltenyi Biotech. In certain aspects, the activator of WNT signalling may be used at 0.6-10 μM.

In an embodiment, the method does not comprise contacting the plurality of stem cells with an activator of fibroblast growth factor (FGF) family signalling simultaneously with the at least one activator of retinoic acid (RA) signalling.

In an embodiment, the activator of FGF signalling is FGF8a, the splice variant of the fibroblast growth factor 8 gene product which gives rise to a protein with a predicted molecular mass of 21 kDa.

In a preferred embodiment, the at least one activator of Retinoic Acid (RA) signalling is selected from the group comprising: a retinoic acid analogue; a RARα agonist; a RARβ agonist; a RARγ agonist; and an RXR agonist.

By “analogue” we include compounds that have similar physical, chemical, biochemical, or pharmacological properties as the subject compound.

By an RARα, RARβ, RARγ and/or RXR “agonist” we include any compound/molecule that potentiate, induce or enhance RARα, RARβ, RARγ and/or RXR signalling. Such compounds/molecules may be involved in signalling downstream of retinoic acid, or be small molecule agonists. Methods for testing if a compound/molecule is capable of inducing or enhancing the activity of a signalling pathway are known to the skilled person.

Non-limiting examples of a RARα agonist; a RARβ agonist; a RARγ agonist; and an RXR agonist include: CD 3254, Docosahexaenoic acid, Fluorobexarotene, LG 100268, SR 11237, AC 261066, AC 55649, Adapalene, AM 580, AM 80, BMS 753, BMS 961, CD 1530, CD 2314, CD 437, Ch 55, TTNPB. In a preferred embodiment, the agonist is a selective agonist (for example, an agonist is said to be selective if it exhibits a greater selectivity for RARα than RARβ.

In a preferred embodiment, the at least one activator of Retinoic Acid (RA) signalling is selected from the group comprising: retinoic acid, all-trans retinoic acid; AM 580; TTNPB; Ch 55; CD437; BMS 961; BMS 753; AM 80; CD 2314; AC 261066; AC 55649; CD 1530; Adapalene; Tazarotenic Acid; Tazarotene; EC 19; EC23; or a functional analogue, isomer, metabolite, or derivative thereof.

It will be appreciated that 9-cis-Retinoic acid (9cRA) is an isomer of all-trans-retinoic acid (ATRA).

The CYP26 family of genes (CYP26A1, CYP26B1, CYP26C1) encode enzymes of the cytochrome p450 family that metabolize RA through oxidation (Thatcher, J. E. & Isoherranen, N. Expert Opin. Drug Metab. Toxicol. 5, 875-86 (2009)). CYP26A1 is expressed by the rostral-most neuroectoderm and contributes to prevent a rostral extension of HB identity at early stages of neural development (reference 22). Also, in AP-patterning of the HB, negative feedback regulation of RA signaling by self-enhanced degradation via induction by CYP26 proteins is important for shaping RA gradients and to buffer for fluctuations of RA levels (White, R. J. & Schilling, T. F. Dev. Dyn. 237, 2775-2790 (2008) and Schilling, T. F., Nie, Q. & Lander, A. D. Curr. Opin. Genet. Dev. 22, 562-569 (2012)).

Without being bound by theory the inventors hypothesize that Tazarotenic Acid; Tazarotene and endogenous retinoids 9-cis and 13-cis and ATRA are degradable by CYP26. Other synthetic retinoids, such as EC23, are not degradable by CYP26 enzymes.

As can be seen from the accompanying Examples, the inventors tested four degradable RA analogues (all-trans, 9-cis, 13-cis RA Tazarotenic Acid) and all could induce ventral midbrain dopaminergic (vMB) progenitor cells characterised as LMX1A+/NKX2.1− after a 48-hour treatment (i.e. 0 to 2 DDC) FIG. 9b (Supplementary FIG. 3b). This vMB inductive response was lost when CYP26 activity was blocked. Non-degradable EC23 could not induce vMB progenitor cells with a 48 hour treatment but could induce LMX1A+/NKX2.1− progenitor cells with a one day treatment at lower EC23 concentrations (i.e. 0 to 1 DDC). As discussed above, the skilled person can use known techniques such as titration in order to determine the effective amount of an activator of Retinoic Acid (RA) signalling needed in the method of the present invention.

Thus, activators of Retinoic Acid (RA) signalling that function by activating either RARα, RARβ, RARγ, and/or RXR receptor and which are subject to CYP26 degradation, and also those non-degradable analogues thereof, are included herein.

In an embodiment, the at least one activator of Retinoic Acid (RA) signalling is degradable by CYP26 enzymes.

In a preferred embodiment, the at least one activator of Retinoic Acid (RA) signalling is selected from the group comprising: retinoic acid; and all-trans retinoic acid, such as 9-cis RA and 13-cis RA, and Tazarotenic acid.

There are three stereoisomeric forms of RA, all-trans retinoic acid (ATRA), 9-cis retinoic acid (9cRA) and 13-cis retinoic acid (13cRA), which show different binding affinities to the retinoic acid receptors. ATRA and 13cRA can only bind efficiently to RAR, but 9cRA is a ligand for both nuclear receptors RAR and RXR. All-trans retinoic acid and retinoic acid are used interchangeably herein.

Tazarotenic acid, an active metabolite of tazarotene, is a potent and selective agonist of the retinoid receptor (RAR) that binds to RARα, RARβ, and RARγ. Tazarotenic acid relatively selective activates RARβ and RARγ. Tazarotenic acid is a first xenobiotic substrate of human retinoic acid hydroxylase CYP26A1 and CYP26B1.

9-cis-Retinoic acid (9cRA) is an isomer of all-trans-retinoic acid (ATRA), both of which are lipid molecules synthesized from a common precursor, vitamin A. 9cRA is a potent agonist for retinoid X receptor (RXR) and retinoic acid receptor (RAR). It has neurotrophic functionality, promotes neuronal differentiation and may have therapeutic potential in treating stroke. It also regulates cytokine secretion and lymphocyte proliferation. 9cRA favours the dopamine cells survival and induces neuroprotection in neurodegenerative disorder like Parkinson's disease. It elicits anti-inflammatory function and stimulates mast cells and inhibits interleukin 4 and 5 expression levels. 9cRA is in clinical trial phase II for treating refractory cancer.

13-cis-Retinoic acid (13cRA) is an isomer of all-trans-retinoic acid (ATRA), and has anti-inflammatory and anti-tumor action. The action of RA is mediated through RAR-β and RAR-α receptors. RA attenuates iNOS expression and activity in cytokine-stimulated murine mesangial cells. It induces mitochondrial membrane permeability transition, observed as swelling and as a decrease in membrane potential, and stimulates the release of cytochrome c implicating mechanisms through the apoptosis pathway. These activities are reversed by EGTA and cyclosporin A. RA also increases MMP-1 protein expression partially via increased transcription.

In a specific, non-limiting embodiment, the cells are contacted with at least one inhibitor of TGFβ/Activin-Nodal signalling (i.e. a first SMAD inhibitor), for example, SB431542 at a concentration of about 5 μM for about 7 days (i.e. day 0 to 7 DDC); at least one inhibitor of BMP signalling (i.e. a second SMAD inhibitor), for example, DMH1 at a concentration of about 250 nM for about 7 days (i.e. from 0 to 7 DDC); at least one activator of Hh signalling, for example, SAG 1.3 at a concentration of about 300 nM; for about 9 days (i.e. from 0 to 9 DDC), or for about 8 days (i.e. from 1 to 9 DDC); and at least one activator of Retinoic Acid (RA) signalling, for example, EC23 at a concentration of about 20 nM for about 1 day (i.e. from 0 to 1 DDC, or from 1 to 2 DDC, or from 2 to 3 DDC, or from 3 to 4 DDC.

In a specific, non-limiting embodiment, the cells are contacted with at least one inhibitor of TGFβ/Activin-Nodal signalling (i.e. a first SMAD inhibitor), for example, SB431542 at a concentration of about 5 μM for about 7 days (i.e. from 0 to 7 DDC); at least one inhibitor of BMP signalling (i.e. a second SMAD inhibitor), for example, DMH1 at a concentration of about 250 nM for about 7 days (i.e. from 0 to 7 DDC); at least one activator of Hh signalling, for example, SAG 1.3 at a concentration of about 300 nM; for about 9 days (i.e. from 0 to 9 DDC), or for about 8 days (i.e. from 1 to 9 DDC); and at least one activator of Retinoic Acid (RA) signalling, for example, ATRA at a concentration of about 300 nM for about 2 days (i.e. from 0 to 2 DDC, or from 1 to 3 DDC, or from 2 to 4 DDC).

In an embodiment, the cells are contacted with the activators and inhibitors described herein at a concentration and for a time effective to increase a detectable level of expression of one or more of LMX1A, LMX1B, FOXA2, and OTX2 in the cells.

In a preferred embodiment, the at least one activator of Retinoic Acid (RA) signalling is derived from an exogenous source.

By “exogenous source” we include that the at least one activator of Retinoic Acid (RA) signalling is introduced from or produced outside the organism (stem cell) or system. In other words, the at least one activator of Retinoic Acid (RA) signalling is not from an endogenous source, i.e. not produced or synthesized within the organism (stem cell) or system.

In a preferred embodiment, culturing the stem cells under conditions sufficient to cause differentiation of said stem cells to produce a cell population comprising ventral midbrain dopaminergic progenitor cells takes place in a two-dimensional and/or three-dimensional cell culture.

In an embodiment, cells may be cultured in a two-dimensional (2D) cell culture. This type of cell culture is well-known to the person skilled in the art. In two-dimensional cell culture cells are grown on flat plastic dishes such as Petri dish, flasks and multi-well plates. Biologically derived matrices (e.g. fibrin, collagen and described herein) and synthetic hydrogels (e.g. PAA, PEG) can be used to facilitate 2D cell culture.

By “three-dimensional cell culture” or “3D cell culture” we include that cells are grown in an artificially created environment in which cells are permitted to grow or interact with its surroundings in all three dimensions. Conditions for 3D cell culture are known in the art. For example, in order to achieve the three-dimensional property of the cell culture, cells are grown or differentiated in matrices or scaffolds. In principle, suitable matrices or scaffolds, which can be used in three-dimensional cell cultures are known to the skilled person. Such matrices or scaffolds can therefore be any matrix or scaffold. For example, the matrix or scaffold can be an extracellular matrix comprising either natural molecules or synthetic polymers.

In a preferred embodiment, the cell population comprises a therapeutically effective amount of ventral midbrain dopaminergic progenitor cells.

By “therapeutically effective amount” we include an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the neurodegenerative disorder such as Parkinson's Disease, or otherwise reduce the pathological consequences of the neurodegenerative disorder such as Parkinson's Disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

In an embodiment, the method further comprises differentiating the population comprising ventral midbrain dopaminergic progenitor cells into mesencephalic dopaminergic neurons.

As described in the accompanying Examples, ventral midbrain dopaminergic progenitor cells were derived from hPSCs within 7-9 days (7-9 DDC). These day 7 cells co-express FOXA2 and LMX1A. These day 9 cells co-express FOXA2, LMX1A, and OTX2 and maintain expression of these markers also at later progenitor stages. As the vMB DA progenitors differentiate into post-mitotic neurons they begin to express the pan neuronal marker Tuj1 and, subsequently, the DA neuron transmitter regulator, NURR1. As described in the accompanying Examples, the inventors observed the presence of TuJ1+ neurons at 12 DDC using immunocytochemical analyses, indicating early initiation of neurogenesis. Within 14 DDC about 80% of the cell population derived from hPSCs are ventral midbrain dopaminergic progenitor cells expressing FOXA2, LMX1A, LMX1B and OTX2.

Ventral midbrain progenitors which co-express FOXA2 and LMX1A, can be contacted with additional small molecules to induce further differentiation into mature mesencephalic dopaminergic neurons positive for TH, FOXA2, and LMX1A by 30 DDC.

In an embodiment, differentiating the population comprising ventral midbrain dopaminergic progenitor cells into mesencephalic dopaminergic neurons comprises further contacting the vMB progenitors with DA neuron lineage specific activators and/or inhibitors, including but not limited to, brain-derived neurotrophic factor (BDNF), glial cell-derived neurotrophic factor (GDNF), ascorbic acid (AA), and a gamma-secretase inhibitor such as DAPT (which is also known as (2S)—N-[(3,5-Difluorophenyl)acetyl]-L-alanyl-2-phenyl]glycine 1,1-dimethylethyl ester). Herein, this process is termed “terminal differentiation”.

In an embodiment, DA neuron lineage specific activators and/or inhibitors are comprised within Neurobasal Plus Medium comprising a B27 Plus supplement also referred to as “B27+ medium”), termed a “terminal differentiation medium” (ThermoFisher; A3653401). It will be appreciated that neurons can be maintained in any medium suitable for supporting neurons in the culture known to the skilled person.

In an embodiment, the ventral midbrain progenitors are contacted with BDNF at a concentration of between about 1 and 50 ng/mL, or between about 5 and 40 ng/mL, or between about 5 and 30 ng/mL, or between about 10 and 20 ng/mL. In certain embodiments, the cells are contacted with BDNF at a concentration of about 10 ng/mL.

In an embodiment, the ventral midbrain progenitors are contacted with GDNF at a concentration of between about 1 and 50 ng/mL, or between about 5 and 40 ng/mL, or between about 5 and 30 ng/mL, or between about 10 and 20 ng/mL. In certain embodiments, the cells are contacted with BDNF at a concentration of about 10 ng/mL.

In an embodiment, the ventral midbrain progenitors are contacted with AA at a concentration of between about 50 and 500 μM, or between about 100 and 400 μM, or between about 150 and 300 μM, or between about 180 and 250 μM. In certain embodiments, the cells are contacted with AA at a concentration of about 200 μM.

In an embodiment, the ventral midbrain progenitors are contacted with DAPT at a concentration of between about 1 and 100 μM, or between about 5 and 50 μM, or between about 10 and 20 μM. In certain embodiments, the cells are contacted with AA at a concentration of about 10 μM.

In an embodiment, prior to terminal differentiation, ventral midbrain progenitors were re-plated again between days 16 and 25 (i.e. at 16-25 DDC) to avoid too high densities of cultures and detachment of cells.

In a specific non-limiting embodiment, for terminal in vitro differentiation into dopaminergic neurons, ventral midbrain progenitors are dissociated at 23 or 24 DDC and plated on a coated surface in B27+ medium supplemented with BDNF (10 ng/ml) and GDNF (10 ng/ml) (Miltenyi Biotech), Ascorbic acid (0.2 mM) (Sigma), and optionally 10 μM DAPT (Miltenyi Biotech) until the desired maturation stage has been reached.

In an embodiment, the ventral midbrain progenitors are contacted with the DA neuron lineage specific activators and/or inhibitors for at least about 2, 3, 4, 5, 6, 7, 8, 9, 10, 14 or more days, for example, throughout terminal differentiation from ventral midbrain progenitors into mature dopaminergic neurons. In an embodiment, ventral midbrain progenitors are contacted with BDNF, GDNF and AA throughout terminal differentiation. In an embodiment, a gamma-secretase inhibitor (such as DAPT) is only contacted with the ventral midbrain progenitors for about 7 days.

The developmental origin of mesencephalic dopaminergic neurons has been found to differ from other neurons, as they do not originate from PAX6+ neuroepithelial progenitor cells, but from the FOXA2+/LMX1A+ ventral midbrain progenitors.

By “mesencephalic dopaminergic neurons” or “mesencephalic dopamine neurons” we refer to specialized cells that at least partially adopt a characteristic neuronal morphology in culture, express one or more mesencephalic dopaminergic neuron markers (e.g. tyrosine hydroxylase (TH)), produce and/or release dopamine; and/or acquire the electrophysiological properties typical of midbrain dopamine neurons. Optionally, the mesencephalic dopaminergic neurons additionally express one or more of Nuclear receptor related 1 (NURR1); Paired Like Homeodomain 3 (PITX3), GIRK2, vesicular monoamine transporter (VMAT2) and synaptophysin. The term “midbrain dopaminergic neurons” may be used interchangeably.

Functional maturation of mDA neurons in vitro can be monitored by production and release of dopamine (DA) and by determining the time when dopamine neurons acquire spontaneous action potentials, evoked action potentials as well as voltage-dependent Na+ and K+ currents. Dopamine neurons derived by the method of the invention show both spontaneous and evoked action potentials that could be recorded at 40 DDC (see FIG. 4n). Without being bound by theory, these data suggest that RA-based differentiation results in the generation of mDA neurons exhibiting mature functional features within 40 days of culture (i.e. day 40 DDC). As shown in the accompanying Examples, dopaminergic neurons also expressed the mature neuronal marker synaptophysin and the monoaminergic marker vesicular monoamine transporter (VMAT2). A subset of cells expressed GIRK2 or CALBINDIN indicating the presence of both A9- and A10-like subtypes of midbrain dopamine neurons. Neurons showed significant increase of neurite outgrowth and complexity between day 30-40 of culture (30-40 days DDC).

In a preferred embodiment, the mesencephalic dopaminergic neurons express one or more of forkhead box protein A2 (FOXA2), LIM homeobox transcription factor 1 alpha (LMX1A), LIM homeobox transcription factor 1 beta (LMX1B), Orthodenticle homeobox 2 (OTX2), Nuclear receptor related 1 (NURR1); Paired Like Homeodomain 3 (PITX3), GIRK2, vesicular monoamine transporter (VMAT2), synaptophysin, and Tyrosine hydroxylase (TH).

“Tuj1” also known as “13111 Tubulin” we include a protein that in humans is encoded by the TUBB3 gene. The protein 13111 Tubulin (TuJ1) is present in newly generated immature post-mitotic neurons and differentiated neurons. Human Tuj can comprise sequence as shown in the Uniprot No. Q13509. The term Tuj1 encompasses any Tuj1 nucleic acid molecule or polypeptide and can also comprise fragments or variants thereof. The skilled person knows how to detect Tuj1, and such methods are also described in the accompanying Examples.

“NURR1” is a protein that in humans is encoded by the NR4A2 gene, and is a member of the nuclear receptor family of 31 intracellular transcription factors. NURR1 plays a key role in the maintenance of the dopaminergic system of the brain. Human NURR1 can comprise a protein sequence such as depicted by Uniprot No. P43354. The term NURR1 encompasses any NURR1 nucleic acid molecule or polypeptide and can also comprise fragments or variants thereof. The skilled person knows how to detect NURR1. Such methods are also described in the accompanying Examples.

“GIRK2” (see FIG. 5g), is a marker enriched in A9-type DA neurons.

“Pitx3” is also a specific mDA neuronal marker, and has exclusive expression in mDA neurons and their postmitotic precursors. The last stage in mDA neuronal differentiation proceeds as the Pitx3+ cells and the Th+ cells migrate ventrally. Human Pitx3 can comprise a protein sequence such as depicted by Uniprot No. O75364. The term Pitx3 encompasses any Pitx3 nucleic acid molecule or polypeptide and can also comprise fragments or variants thereof. The skilled person knows how to detect Pitx3. Such methods are also described in the accompanying Examples.

“TH” refers to Tyrosine hydroxylase/tyrosine 3-monooxygenase/tyrosinase, a protein that in humans is encoded by the TH gene. TH is the enzyme responsible for catalyzing the conversion of the amino acid L-tyrosine to L-3,4-dihydroxyphenylalanine (L-DOPA). Human TH can comprise a protein sequence of Uniprot No. P07101. The term TH encompasses any TH nucleic acid molecule or polypeptide and can also comprise fragments or variants thereof. The skilled person knows how to detect TH. Such methods are also described in the accompanying Examples.

In a preferred embodiment, a population comprising differentiated mesencephalic dopaminergic neurons is obtainable within about 30-40 days after first contacting the plurality of stem cells with the at least one activator of Retinoic Acid (RA) signalling.

As discussed above, differentiated dopamine neurons derived by the method of the invention show both spontaneous and evoked action potentials that could be recorded at 40 DDC (see FIG. 4n). Without being bound by theory, these data suggest that RA-based differentiation results in the generation of mDA neurons exhibiting mature functional features within 40 days of culture (i.e. 40 DDC).

In an embodiment, the cells are contacted with the DA neuron lineage specific activators and/or inhibitors described herein at a concentration and for a time effective to increase a detectable level of expression of one or more of marker of a DA neuron, for example, forkhead box protein A2 (FOXA2), LIM homeobox transcription factor 1 alpha (LMX1A), LIM homeobox transcription factor 1 beta (LMX1B), Orthodenticle homeobox 2 (OTX2), Nuclear receptor related 1 (NURR1); Paired Like Homeodomain 3 (PITX3), GIRK2, vesicular monoamine transporter (VMAT2), synaptophysin, and Tyrosine hydroxylase (TH).

As shown in the accompanying Examples, TH+ dopaminergic neurons expressed mDA neuron markers LMX1A, LMX1B, FOXA2, NURR1, and OTX2 at 30-35 DDC (FIG. 4e).

In a preferred embodiment, a population comprising differentiated mesencephalic dopaminergic neurons is obtainable within about 30-40 DDC, such as about within 31 DDC, such as about within 32 DDC, such as about within 33 DDC, such as about within 34 DDC, such as about within 35 DDC, such as about within 36 DDC, such as about within 37 DDC, such as about within 38 DDC, such as about within 39 DDC, such as about within 40 DDC.

In a preferred embodiment, within about 30-40 days after first contacting the plurality of stem cells with the at least one activator of Retinoic Acid (RA) signalling, the total cell population comprises at least 60%, such as at least 70%, or at least 80% mesencephalic dopaminergic neurons.

In a preferred embodiment, the total cell population comprises at least 65% mesencephalic dopaminergic neurons such as at least 66%, 67%, 68%, 69%, 70%, 71%, 72%, 73%, 74%, 75%, 76%, 77%, 78%, 79%, or such as at least 80% mesencephalic dopaminergic neurons.

In a preferred embodiment, within about 30-40 days after first contacting the plurality of stem cells with the at least one activator of Retinoic Acid (RA) signalling, the neuronal cell population comprises at least 70%, such as at least 80% or at least 90% mesencephalic dopaminergic neurons.

In a preferred embodiment, the neuronal cell population comprises at least 70%, 71%, 72%, 73%, 74%, 75% mesencephalic dopaminergic neurons such as at least 76%, 77%, 78%, 79%, 80%, 81%, 82%, 83%, 84%, 85%, 86%, 87%, 88%, 89%, or such as at least 90% mesencephalic dopaminergic neurons.

By “total cell population” we include all cells in the population which are positive for the nuclear marker DAPI. By “neuronal cell population” we include all neurons in the cell population, such as all cells in the population which are positive for the neuronal marker HuCD. As shown in the accompanying Examples, about 80% of all neurons are TH+(i.e. mesencephalic dopaminergic neurons) at 35 DDC which corresponds to about 65% of total DAPI+ cells (FIG. 4i, j).

In one aspect, the present invention provides a method of screening for a candidate drug comprising (a) providing a population of ventral midbrain dopaminergic progenitor cells obtainable or obtained by the method of the invention, or providing a population of differentiated mesencephalic dopaminergic neurons obtainable or obtained by the method of the invention; (b) contacting the population with a candidate drug; and (c) determining the effect of the candidate drug on the cell population.

In one aspect, the present invention provides a method of screening for a candidate drug comprising (a) providing a population of ventral midbrain dopaminergic progenitor cells obtainable or obtained by the method of the invention; (b) contacting the population with a candidate drug; and (c) determining the effect of the candidate drug on the cell population.

vMB progenitors produced by the methods of this invention can be used to screen for factors (such as small molecule drugs, peptides, and polynucleotides) or environmental conditions (such as culture conditions or manipulation) that promote and/or enhance differentiation and maturation of neurons in culture.

In some applications, vMBs may be used to screen factors that promote maturation of the progenitor cells along the neural lineage, or promote proliferation and maintenance of such cells in long-term culture. For example, candidate neural maturation factors or growth factors are tested by contacting them with the vMBs, and then determining any phenotypic change that results, according to desirable criteria for further culture and use of the cells.

In one aspect, the present invention provides a method of screening for a candidate drug comprising (a) providing a population of differentiated mesencephalic dopaminergic neurons obtainable or obtained by the method of the invention; (b) contacting the population with a candidate drug; and (c) determining the effect of the candidate drug on the cell population.

In some applications, differentiated mDA may be used to identify molecules that support and/or enhance the survival of mDA neurons, and therefore may be useful for the treatment of neurodegenerative diseases such as Parkinson's Disease.

Particular screening applications of this invention relate to the testing of pharmaceutical compounds in drug research. In certain embodiments, cells produced by the methods described herein may be used as test cells for standard drug screening and toxicity assays (e.g. to identify, confirm, and test for specification of function or for testing delivery of therapeutic molecules to treat a specific disease). Assessment of the activity of candidate pharmaceutical compounds generally involves combining the population of ventral midbrain dopaminergic progenitor cells, or the population of mesencephalic dopaminergic neurons provided in certain aspects of this invention with the candidate compound, determining any change in the morphology, marker phenotype, or metabolic activity of the cells that is attributable to the compound (compared with untreated cells or cells treated with an inert (control) compound), and then correlating the effect of the compound with the observed change. The screening may be done either because the compound is designed to have a pharmacological effect on vMB dopaminergic progenitors or dopaminergic neurons, or because a compound designed to have effects elsewhere may have unintended neural side effects. As will be appreciated, two or more drugs can be tested in combination (by combining with the cells either simultaneously or sequentially), to detect possible drug-drug interaction effects.

In some applications, compounds are screened initially for potential neurotoxicity. Cytotoxicity can be determined in the first instance by the effect on cell viability, survival, morphology, or other techniques known in the art. More detailed analysis is conducted to determine whether compounds affect cell function (such as neurotransmission) without causing toxicity.

In one aspect, the present invention provides a method for providing an enriched population of:
i. ventral midbrain dopaminergic progenitor cells, wherein the method comprises contacting a plurality of stem cells with an effective amount of at least one activator of retinoic acid (RA) signalling, and culturing the stem cells under conditions sufficient to cause differentiation of the stem cells into a cell population comprising ventral midbrain dopaminergic progenitor cells; or
ii. midbrain dopaminergic (DA) neurons wherein the method comprises the method defined in (i) and further comprises differentiating the population comprising ventral midbrain dopaminergic progenitor cells into mesencephalic dopaminergic neurons.

As used herein, the term “enriched population” refers to a population of cells, such as a population of cells in a culture dish, expressing a marker at a higher percentage or amount than a comparison population, for example, contacting a stem cell with at least one activator of RA signalling and at least one activator of Hh signalling (e.g. SAG) results in an enriched population of mesencephalic dopaminergic neurons as compared to contacting a stem cell with at least one activator of WNT signalling and at least one activator of Hh signalling (e.g. SAG) at 17 DDC and 21 DDC as demonstrated by immunocytochemistry (FIG. 4c).

In other examples, an enriched population is a population resulting from sorting or separating cells expressing one or more markers from cells not expressing the desired marker, such as an FOXA2+ and LMX1A+ enriched population, an A9 enriched population, and the like. It will be appreciated that the cell populations produced by the methods of the invention can be sorted for at least one marker of ventral midbrain dopaminergic progenitor cells or mesencephalic dopaminergic neurons.

In an embodiment, at least 60%, such as at least 65%, 70%, 75%, 80%, 85%, 90%, 95%, 96%, 97%, 98% or 99% of the cells in the enriched population of mesencephalic dopaminergic neurons are positive for a marker of mesencephalic dopaminergic neurons. In an embodiment, an enriched population of mesencephalic dopaminergic neurons comprises at least 1×10², 1×10³, 1×10⁴, 1×10⁵, or 1×10⁶ mesencephalic dopaminergic neurons.

In an embodiment, at least 60%, such as at least 65%, 70%, 75%, 80%, 85%, 90%, 95%, 96%, 97%, 98% or 99% of the cells in the enriched population of ventral midbrain dopaminergic progenitor cells are positive for a marker of ventral midbrain dopaminergic progenitor cells. In further aspects, an enriched population of midbrain DA neurons produced by a method of the embodiments comprises at least 1×10², 1×10³, 1×10⁴, 1×10⁵, or 1×10⁶ ventral midbrain dopaminergic progenitor cells.

In one aspect, the present invention provides a neuronal cell population comprising a therapeutically effective amount of ventral midbrain dopaminergic progenitor cells obtained or obtainable by any of the methods disclosed herein, optionally wherein at least 60%, such as at least 65%, such as at least 70%, such as at least 75%, or at least 80% of the cell population are ventral midbrain dopaminergic progenitor cells.

In a preferred embodiment, at least about 80% of the neuronal cell population express forkhead box protein A2 (FOXA2), LIM homeobox transcription factor 1 alpha (LMX1A), LIM homeobox transcription factor 1 beta (LMX1B) and Orthodenticle homeobox 2 (OTX2).

As described herein, ventral midbrain dopaminergic progenitor cells were derived from hPSCs within 7 days following initial exposure to RA (i.e. on day 7 DDC). These 7 DDC cells co-express FOXA2 and LMX1A. As can be seen in the accompanying Examples, at 14 DDC about 80% of the cell population derived from hPSCs are ventral midbrain dopaminergic progenitor cells co-expressing FOXA2, LMX1A, LMX1B and OTX2 as well as the vMB marker CORIN (FIG. 2g) as determined by immunocytochemistry. Accordingly, it will be appreciated that this is a cell population enriched for ventral midbrain dopaminergic progenitor cells.

In one aspect, the present invention provides a differentiated cell population comprising a therapeutically effective amount of mesencephalic dopaminergic neurons obtained or obtainable by any of the methods disclosed herein, optionally wherein at least 60%, such as at least 65%, such as at least 70%, such as at least 75%, or at least 80% of the total cells are mesencephalic dopaminergic neurons.

As shown in the accompanying Examples, about 80% of all neurons are TH+(i.e. mesencephalic dopaminergic neurons) at 35 DDC which corresponds to about 65% of total DAPI+ cells (FIG. 4i, j). Accordingly, it will be appreciated that this is a cell population enriched for mesencephalic dopaminergic neurons.

In one aspect, the present invention provides the use of at least one activator of Retinoic Acid (RA) signalling for differentiating stem cells into ventral midbrain dopaminergic progenitor cells.

In a preferred embodiment, differentiating stem cells into ventral midbrain dopaminergic progenitor cells is as described herein.

In one aspect, the present invention provides an isolated cell population, comprising a therapeutically effective amount of ventral midbrain dopaminergic progenitor cells.

In one aspect, the present invention provides an isolated cell population, comprising a therapeutically effective amount of ventral midbrain dopaminergic progenitor cells obtained or obtainable by the methods described herein.

In one aspect, the present invention provides an isolated cell population, comprising a therapeutically effective amount of mesencephalic dopaminergic neurons.

In one aspect, the present invention provides an isolated cell population, comprising a therapeutically effective amount of mesencephalic dopaminergic neurons obtained or obtainable by the methods described herein.

By “isolated,” when referring to a material, we include a material that is partially or completely removed from the other material which naturally accompanies it. Therefore, in reference to a cell population, the term “isolated” refers to a cell substantially free from other cell populations accompanying it in vivo.

In one embodiment, said isolated population is derived from a cell population selected from the group consisting of mammals, primates, humans and a patient with a symptom of Parkinson's disease (PD).

In a further embodiment an isolated population of cells is provided in a stable freezing solution comprising viable ventral midbrain dopaminergic progenitor cells, or mesencephalic dopaminergic neurons, or a mixture thereof. In some embodiments, an isolated population of cells in a stable freezing solution is comprised of at least 60%, 65%, 70%, 75%, 80%, 85%, 90% or 95% cells that are positive for FoxA2 and/or Lmx1a expression. In further aspects, a population of the embodiments comprises at least about 40%, 45%, 50%, 55%, 60%, 65%, 70%, 75%, 80%, 85% or 90% viable vMBs after one freeze-thaw cycle.

A stable freezing solution comprises one or more of a cell culture medium, a protease or protease cocktail, stabilizer (e.g., DMSO or glycerol), growth factor, buffers, or extracellular matrix components. In further embodiments, there is provided a sealed vial.

In a preferred embodiment, at least about 80% of the cell population express forkhead box protein A2 (FOXA2), LIM homeobox transcription factor 1 alpha (LMX1A), LIM homeobox transcription factor 1 beta (LMX1B) and Orthodenticle homeobox 2 (OTX2).

In one aspect, the present invention provides a pharmaceutical composition comprising a cell population comprising a therapeutically effective amount of ventral midbrain dopaminergic progenitor cells as described in any aspect or embodiment herein, and/or obtained or obtainable by any of the methods described herein, for use in medicine.

In a preferred embodiment, the pharmaceutical composition further comprising a pharmaceutically acceptable carrier, diluent and/or excipient.

Following differentiation of stem cells into ventral midbrain dopaminergic progenitor cells as described herein, in one embodiment, the cells may be prepared for transplantation. The cells may be suspended in a physiologically acceptable carrier, such as cell culture medium (e.g., Eagle's minimal essential media), phosphate buffered saline, or artificial cerebrospinal fluid (aCSF). The volume of cell suspension to be implanted will vary depending on the site of implantation, treatment goal, and cell density in the solution (Nolbrant, Sara, et al. “Generation of high-purity human ventral midbrain dopaminergic progenitors for in vitro maturation and intracerebral transplantation.” Nature protocols 12.9 (2017): 1962).

Pharmaceutically acceptable carriers and diluents include saline, aqueous buffer solutions, solvents and/or dispersion media. The use of such carriers and diluents is well known in the art. The solution is preferably sterile and fluid. Suitably, the solution is stable under the conditions of manufacture and storage and preserved against the contaminating action of microorganisms such as bacteria and fungi through the use of, for example, parabens, chlorobutanol, phenol, ascorbic acid, or thimerosal. Solutions of the invention can be prepared by incorporating the cells as described herein in a pharmaceutically acceptable carrier or diluent and, as required, other ingredients.

The presently disclosed ventral midbrain dopaminergic progenitor cells and the pharmaceutical compositions comprising said cells can be conveniently provided as sterile liquid preparations, e.g., isotonic aqueous solutions, suspensions, emulsions, dispersions, or viscous compositions, which may be buffered to a selected pH Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the compositions of the presently disclosed subject matter, e.g., a composition comprising the presently disclosed stem-cell-derived precursors, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavouring agents, colours, and the like, depending upon the route of administration and the preparation desired.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin. According to the presently disclosed subject matter, however, any vehicle, diluent, or additive used would have to be compatible with the presently disclosed ventral midbrain dopaminergic progenitor cells.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

It will be appreciated that the components of the compositions should be selected to be chemically inert and will not affect the viability or efficacy of the presently disclosed ventral midbrain dopaminergic progenitor cells.

In a preferred embodiment, the pharmaceutical composition is formulated for transplantation.

In one aspect, the present invention provides a kit for differentiating a plurality of stem cells into ventral midbrain dopaminergic progenitor cells or into mesencephalic dopaminergic neurons in vitro, comprising:
at least one activator Retinoic Acid (RA) signalling;
at least one activator of Sonic Hedgehog (SHH) signalling;
at least one inhibitor of TGFβ/Activin-Nodal signalling; and/or
at least one inhibitor of bone morphogenetic protein (BMP) signalling.

In an embodiment, the kit further comprises one or more substrate for cell adhesion. Non-limiting substrates for cell adhesion include collagen, gelatin, poly-L-lysine, poly-D-lysine, poly-L-ornithine, laminin, vitronectin, and fibronectin and mixtures thereof, such as Matrigel™ or Geltrex, and lysed cell membrane preparations.

In an embodiment, the kit further comprises a plurality of markers of ventral midbrain dopaminergic progenitor cells or mesencephalic dopaminergic neurons.

In one aspect, the present invention provides a kit comprising a therapeutically effective amount of ventral midbrain dopaminergic progenitor cells obtained or obtainable by any of the methods described herein, and one or more dopaminergic neuron lineage specific activators and/or inhibitors.

In a further embodiment, the one or more DA neuron lineage specific activators and/or inhibitors necessary are suitable for terminal differentiation of ventral midbrain dopaminergic progenitor cells into mesencephalic dopaminergic neurons. Such components are described above and include but are not limited to, brain-derived neurotrophic factor (BDNF), glial cell-derived neurotrophic factor (GDNF), ascorbic acid (AA), ascorbic acid (AA), and a gamma-secretase inhibitor such as DAPT (which is also known as (2S)—N-[(3,5-Difluorophenyl)acetyl]-L-alanyl-2-phenyl]glycine 1,1-dimethylethyl ester). The one or more specific activators and/or inhibitors necessary suitable for terminal differentiation may be comprised within a B27+ medium, or provided separately.

In an embodiment, the kit comprises instructions for administering a population of the presently disclosed ventral midbrain dopaminergic progenitor cells or a composition comprising thereof to a subject suffering from a neurodegenerative disease, or disease and/or condition characterised by the loss of midbrain dopaminergic neurons.

In one aspect, the present invention provides a cell population comprising a therapeutically effective amount of ventral midbrain dopaminergic progenitor cells as described in any aspect or embodiment herein, and/or obtained or obtainable by any of the methods described herein, for use in medicine.

In one aspect, the present invention provides a cell population comprising a therapeutically effective amount of ventral midbrain dopaminergic progenitor cells as described in any aspect or embodiment herein, and/or obtained or obtainable by any of the methods described herein, for use in treating or preventing neurodegeneration in a subject and/or a disease and/or condition characterised by the loss of midbrain dopaminergic neurons in a subject.

In one aspect, the present invention provides a method for treating or preventing neurodegeneration in a subject and/or a disease and/or condition characterised by the loss of midbrain dopaminergic neurons in a subject, comprising administering to the subject a cell population comprising a therapeutically effective amount of ventral midbrain dopaminergic progenitor cells as described in any aspect or embodiment herein, and/or obtained or obtainable by any method described herein, in an amount effective to treat or prevent the neurodegeneration in the subject and/or a disease and/or condition characterised by the loss of midbrain dopaminergic neurons in a subject.

In one aspect, the present invention provides use of a cell population comprising a therapeutically effective amount of ventral midbrain dopaminergic progenitor cells as described in any aspect or embodiment herein, and/or obtained or obtainable by any of the methods described herein, for the manufacture of a medicament for treating or preventing neurodegeneration in a subject and/or a disease and/or condition characterised by the loss of midbrain dopaminergic neurons in a subject.

By “neurodegeneration in a subject” we include a progressive loss of neuronal functionality as the result of neurodegenerative processes in a subject. Neurodegeneration can be caused by the selective loss of DA neurons in the substantia nigra of the ventral midbrain. We also include neurodegeneration caused by neurodegenerative disorders including but not limited to Parkinson's disease, Huntington's disease, Alzheimer's disease, and multiple sclerosis.

By a “disease and/or condition characterised by the loss of midbrain dopaminergic neurons” we include any disease and/or condition which displays symptoms caused by the loss of dopamine neurons in a subject. Neuronal loss in the SNpc is currently thought to be caused by mitochondrial damage, energy failure, oxidative stress, excitotoxicity, protein misfolding and their aggregation, impairment of protein clearance pathways, cell-autonomous mechanisms and/or prion-like protein infection. As discussed above, the regions of DA producing neurons are derived from the tegmentum and are called the substantia nigra pars compacta (SNc, A9 group), and the ventral tegmental area (VTA, A10 group). The skilled person is aware that the mesencephalic dopaminergic (mDA) neurons of the SNc play an important role in the control of multiple brain functions. Their axons ascend rostrally into the dorsolateral striatum of the cortex, where they release the neurotransmitter dopamine. mDA neurons of the SNc selectively undergo degeneration in Parkinson's disease (PD), a progressive neurodegenerative disorder characterized by the progressive loss of dopaminergic neurons in the substantia nigra. The loss of mDA neurons leads to a lack of DA in the striatum, which controls voluntary body movements under physiological conditions. Methods of identifying the loss of midbrain dopaminergic neurons are known in the art and include electrophysiology, measuring evoked dopamine release, and behavioural analyses.

In an embodiment the diseases and/or conditions characterised by the loss of midbrain dopaminergic neurons in a subject include neurodegenerative diseases including but not limited to Parkinson's disease, Parkinsonism syndrome, Alzheimer's disease, stroke, amyotrophic lateral sclerosis, Binswanger's disease, Huntington's chorea, multiple sclerosis, myasthenia gravis and Pick's disease. In an embodiment, the neurodegenerative disease is a parkinsonism disease, which refers to diseases that are linked to an insufficiency of dopamine in the basal ganglia, which is a part of the brain that controls movement.

Parkinson's disease is often accompanied with sensory, sleep, and emotional problems. “Parkinsonism”, or a “parkinsonian syndrome” are the main motor symptoms.

Non-limiting examples of parkinsonism syndrome include Lewy body dementia, idiopathic Parkinson disease (PD), progressive supranuclear palsy (PSP), multiple system atrophy (MSA), corticobasal degeneration (CBD), and vascular Parkinsonism (VaP), among other rarer causes of parkinsonism.

In an embodiment, the neurodegenerative disease is Parkinson's disease (PD). Presently, PD is classified into two subtypes, primary or secondary. Primary parkinsonism includes genetic and idiopathic forms of the disease and secondary parkinsonism includes forms induced by drugs, infections, toxins, vascular defects, brain trauma or tumors or metabolic dysfunctions.

Primary motor symptoms of Parkinson's disease include, for example, but not limited to, resting tremor (shaking of hands, arms, legs, jaw, head, tongue, lips, chin are the primary motor symptoms observed in PD), rigidity, bradykinesia (slow movement), and postural instability or impaired balance and coordination. Secondary motor symptoms include stooped posture, a tendency to lean forward, dystonia, fatigue, impaired fine and gross motor coordination, decreased arm swing, akathisia, cramping, drooling, difficulty with swallowing and chewing, and sexual dysfunction. Frequently observed non-motor symptoms in PD patients include depression, insomnia, and cognitive dysfunction.

PD can be diagnosed by the skilled clinician using, for example, diagnostic criteria defined by the International Parkinson and Movement Disorder Society (MDS) Clinical Diagnostic Criteria for Parkinson's disease (MDS-PD Criteria).

By “treating” or “treatment” we include any treatment of a disease in a subject and includes: inhibiting the disease, e.g., preventing engraftment failure and/or relieving the disease, e.g., causing regression of one or more symptoms. For example, treatment may involve the relief of one or more neurological symptom selected from the group comprising resting tremor, rigidity, bradykinesia (slow movement), and postural instability or impaired balance and coordination

Generally, the efficacy of a given treatment can be determined by the skilled artisan. However, a treatment is considered “effective treatment,” as the term is used herein, if any one or all of the signs or symptoms of e.g., tremor, are altered in a beneficial manner, other clinically accepted symptoms are improved, or even ameliorated, e.g., by at least 10% following treatment with a cell population as described herein. Efficacy can also be measured by a failure of an individual to worsen as assessed by hospitalization, need for medical interventions (i.e., progression of the disease is halted), or incidence of engraftment failure. Methods of measuring these indicators are known to those of skill in the art and/or are described herein. In the context of the present invention, efficacy can be assessed in animal models of Parkinson's Disease, for example, by performing behavioural tests, such as step tests and cylinder tests. Efficacy of treatment can be determined by assessing physical indicators of, for example, DA neuron engraftment/transplant, such as, neurological symptoms including resting tremor, rigidity, bradykinesia (slow movement), and postural instability or impaired balance and coordination.

By “preventing” or “prevention” we generally include reducing or decreasing the occurrence of the disorder or condition in the subject, or delaying the onset of one or more symptoms of the disorder or condition. In the context of the present invention, prevention may include preventing degeneration, i.e. reducing the loss of cells, or reducing impairment of cell function, e.g., release of dopamine in the case of dopaminergic neurons. Prevention also includes the reduction or decrease of the severity of neurological conditions deriving from loss of dopaminergic progenitors and/or loss of neurons of the substantia nigra.

The subject can be a vertebrate, more preferably a mammal. Mammals include, but are not limited to, farm animals including pigs, primates, dogs, horses, and rodents. A mammal can be a human, dog, cat, cow, pig, mouse, rat etc. Thus, in one embodiment, the subject is a vertebrate. Preferably, the subject is a human subject. The subject can be a subject suffering from a neurodegenerative disease such as Parkinson's disease. In particular, the subject may be a subject comprising the LRRK2-G2019S mutation, which is associated with familial Parkinson's disease. The subject can also be a subject not suffering from a neurodegenerative disease such as Parkinson's disease.

Ventral midbrain dopaminergic progenitor or dopaminergic neurons can be administered to a subject either locally or systemically. Methods of administration are known in the art. If the subject is receiving cells derived from his or her own cells, this is called an autologous transplant; such a transplant is less likely to lead to rejection.

Exemplary methods of administering stem cells or differentiated cells to a subject, particularly a human subject, include injection or transplantation of the cells into target sites (e.g., striatum and/or substantia nigra) in the subject. The vMB progenitors and/or DA neurons can be inserted into a delivery device which facilitates introduction, by injection or transplantation of the cells into the subject. Such delivery devices include tubes, e.g., catheters, for injecting cells and fluids into the body of a recipient subject. In a preferred embodiment, the tubes additionally have a needle, e.g., a syringe, through which the cells of the invention can be introduced into the subject at a desired location. The vMB progenitors can be inserted into such a delivery device, e.g., a syringe, in different forms. For example, the cells can be suspended in a solution, or alternatively embedded in a support matrix when contained in such a delivery device.

Support matrices in which the vMB progenitors can be incorporated or embedded include matrices that are recipient-compatible and that degrade into products that are not harmful to the recipient. The support matrices can be natural (e.g., collagen, etc.) and/or synthetic biodegradable matrices. Synthetic biodegradable matrices include synthetic polymers such as polyanhydrides, polyorthoesters, and polylactic acid.

As can be seen in the accompanying Examples, parkinsonian was induced in rats by unilaterally injecting 6-hydroxydopamine (6-OHDA) into the medial forebrain bundle for complete lesion of the nigrostriatal dopaminergic pathway in the right hemisphere. hPSC-derived vMB-progenitors were isolated at day 14 of differentiation (14 DDC) and were dissociated into single cell suspension with accutase, resuspended in a physiological buffer such as (HBSS buffer+DNase) to a concentration of 37,500-75,000 cells per μl and grafted into the host rat striatum on the 6-0H DA-lesioned side.

In a preferred embodiment, the subject exhibits at least one neurological symptom, wherein the neurological symptom is selected from the group comprising of: resting tremor, rigidity, bradykinesia (slow movement), and postural instability and/or impaired balance and coordination.

In a preferred embodiment, the subject shows a reduction of at least one of said neurological symptom.

In a preferred embodiment, the population comprising ventral midbrain dopaminergic progenitor cells is administered by transplantation to a subject under conditions that allow in vivo engraftment of the population of cells.

In one aspect, the methods described herein provide a method for enhancing engraftment of vMB progenitor cells or DA neurons in a subject. In one embodiment, the subject can be a mammal. In another embodiment, the mammal can be a human, although the invention is effective with respect to all mammals.

Transplantation can be allogeneic (i.e. between genetically different members of the same species), autologous (i.e. transplantation of an organism's own cells or tissues), syngeneic (i.e. between genetically identical members of the same species (e.g., identical twins)), or xenogeneic (i.e. between members of different species).

Transplantation of the cells described in any aspect or embodiment of the invention into the brain of the patient with a neurodegenerative disease results in replacement of lost, non-, and/or dysfunctional DA neurons. The cells are introduced into a subject with a neurodegenerative disease in an amount suitable to replace the lost and/or dysfunctional DA neurons such that there is an at least partial reduction or alleviation of at least one adverse effect or symptom of the disease. The cells can be administered to a subject by any appropriate route that results in delivery of the cells to a desired location in the subject where at least a portion of the cells remain viable.

It is preferred that at least about 5%, preferably at least about 10%, more preferably at least about 20%, yet more preferably at least about 30%, still more preferably at least about 40%, and most preferably at least about 50% or more of the cells remain viable after transplantation into a subject. The period of viability of the cells after administration to a subject can be as short as a few hours, e.g., 24 hours, to a few days, to as long as a few weeks to months. It will be appreciated that any transplantation method known to the skilled person that can be used to deliver the cells to a subject may be used (Parmar, Malin. “Towards stem cell based therapies for Parkinson's disease.” Development 145.1 (2018): dev156117).

In one aspect, the present invention provides a cell population comprising a therapeutically effective amount of ventral midbrain dopaminergic progenitor cells as described in any aspect or embodiment herein, and/or obtained or obtainable by any of the methods described herein, for use in transplanting into a subject in need thereof.

In one aspect, the present invention provides a method for transplanting a cell population comprising a therapeutically effective amount of ventral midbrain dopaminergic progenitor cells as described in any aspect or embodiment herein, and/or obtained or obtainable by any of the methods described herein into a subject in need thereof.

In one aspect, the present invention provides use of a cell population comprising a therapeutically effective amount of ventral midbrain dopaminergic progenitor cells as described in any aspect or embodiment herein, and/or obtained or obtainable by any of the methods described herein, for the manufacture of a medicament for transplanting into a subject in need thereof.

In a preferred embodiment, the subject has or is at risk of a neurodegenerative disease selected from the group comprising: Parkinson's disease, Parkinsonism syndrome, Alzheimer's disease, stroke, amyotrophic lateral sclerosis, Binswanger's disease, Huntington's chorea, multiple sclerosis, myasthenia gravis and Pick's disease.

In an embodiment, the Parkinson's disease is sporadic Parkinson's disease, familial Parkinson's disease, for example comprising the LRRK2-G2019S mutation, and/or autosomal recessive early-onset Parkinson's disease.

In one aspect, the present invention provides a method of differentiating, a population for use, use of a population, a method of treating, or a kit substantially as described herein, with reference to the accompanying description, examples and drawings.

The invention will now be described further by reference to the following Figures and Examples.

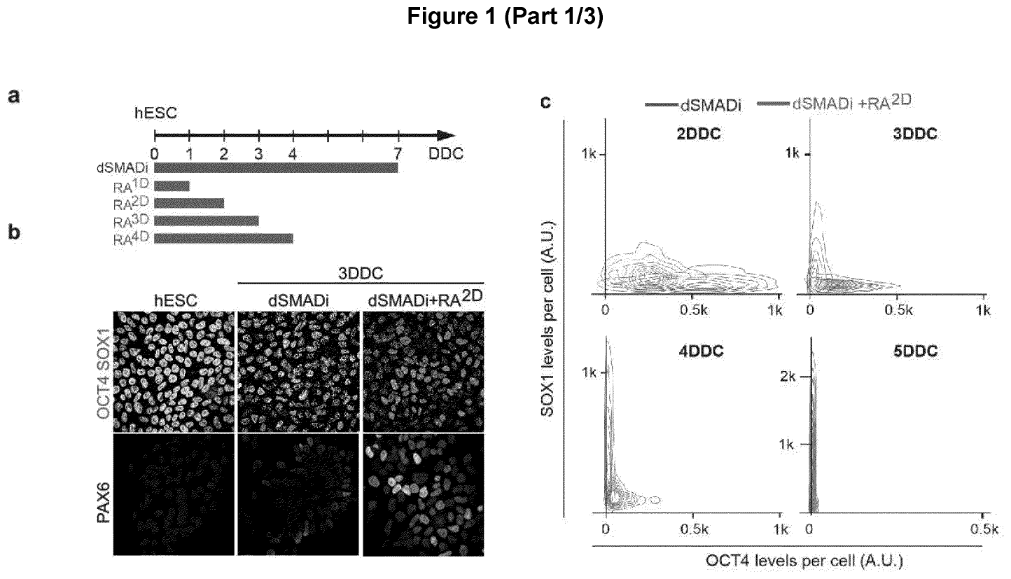

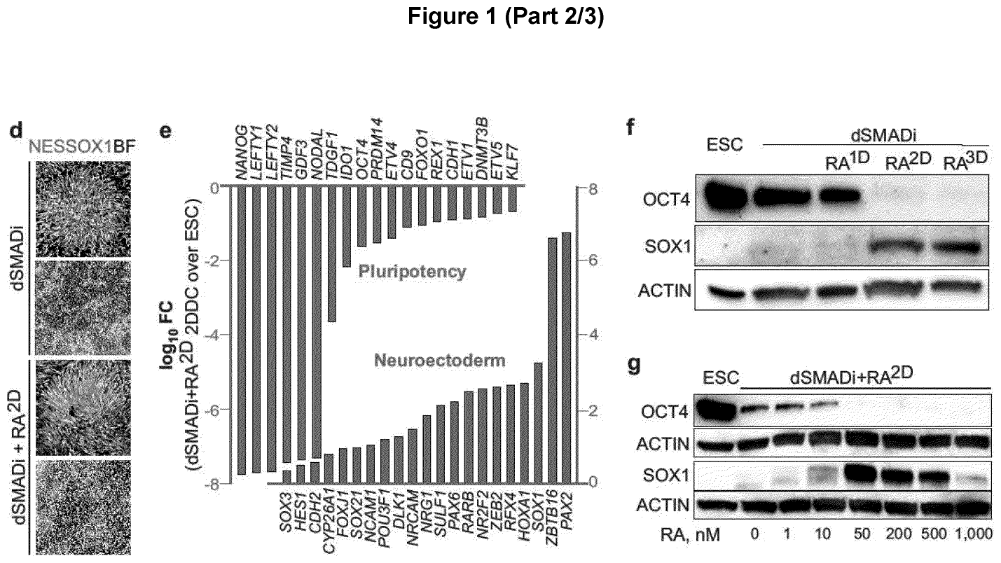

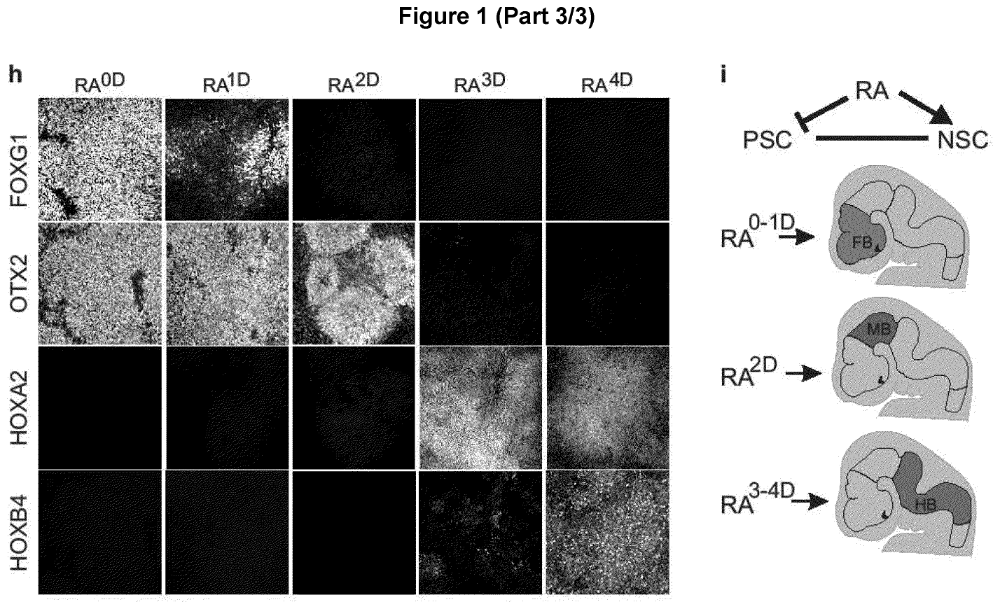

FIG. 1: A two-day RA-pulse results in a rapid induction of NSCs expressing a MB-like identity. (a) Schematic of hPSCs differentiation with timeline of treatment with dSMADi and RA. (b) Immunocytochemistry of ESC and 3-day cultures differentiated in dual SMAD inhibitors (dSMADi) or in dSMADi with a two-day RA-pulse (RA^{2D}) for the pluripotency marker OCT4 and the neuronal markers SOX1 and PAX6. (c) Density plot of single cell expression of SOX1 and OCT4 in dSMADi or dSMADi with a RA^{2D} pulse at indicated days in differentiation conditions (DDC). (d) Immunocytochemistry for neural progenitor markers NES and SOX1 and bright-field (BF) images of cultures at 7 DDC differentiated in the indicated conditions. (e) Changes in expression of genes associated with pluripotency or neuroectodermal fate in dSMADi+RA^{2D}-pulsed cultures at 2 DDC relative to ESCs. (f) Western blot for OCT4 and SOX1 of ESC and 3 DDC cultures grown in dSMADi-conditions and treated with indicated concentrations of RA for 2 days. (g) Western blot for OCT4 and SOX1 of ESC and 3 DDC cultures grown in indicated differentiation conditions. (h) Immunocytochemistry for the markers identifying forebrain (FOXG1), forebrain and midbrain (OTX2), hindbrain (HOXA2), and caudal hindbrain (HOXB4) regions in 9 DDC cultures differentiated in dSMADi conditions and treated with a RA-pulse as indicated. (i) Summary of the effects of RA-pulse duration on NSC's regional identity.

A.U., arbitrary units. FB, forebrain. MB, midbrain. HB, hindbrain.

FIG. 2: Specification of NSC with ventral midbrain identity using RA and SHH signaling. (a) Schematic of hPSCs differentiation with timeline of treatment with RA and activator of SHH signaling (SAG) (top). All cultures were differentiated in dSMADi conditions as indicated in FIG. 1a. Immunocytochemistry for indicated markers at 9 DDC in cultures differentiated in dSMADi+SAG-condition and pulsed with RA for 1-, 2- or 4-days (bottom). (b) Relative gene expression (FPKM) of indicated genes in 9 DDC cultures differentiated in dSMADi+SAG-conditions and treated with RA for the indicated time. (c) Heatmap and hierarchical clustering of differentially expressed genes of 9 DDC cultures differentiated in dSMADi+SAG-condition and treated with RA for the indicated time. (d) Schematic of gene expression profiles defining distinct ventral progenitors in the diencephalon (Di), midbrain (MB) and hindbrain (HB) positions. (e) Expression of genes associated with the indicated regional progenitors at 14 DDC in cultures differentiated in dSMADi+SAG+RA^{2D} condition. (f) Immunocytochemistry for β-CATENIN (left) and boxplots of β-CATENIN nuclear levels (right) in cultures at 2 DDC differentiated in dSMADi+SAG-condition (control) and treated with RA or CHIR99021. Boxplot, whiskers define 5^{th} and 95^{th} percentile. Asterisks, Student t test; **p<0.01, ***p<0.001. (g) Immunocytochemistry for the indicated markers in cultures at 14 DDC differentiated in dSMADi+SAG+RA^{2D} condition.

FIG. 3: A RA-CYP26 regulatory loop is central for robust RA-mediated patterning response. (a-c) Effect of changes in RA (a) or CHIR99221 (b) concentration on the expression of markers defining ventral forebrain (NKX2.1+LMX1A+), midbrain (NKX2.1⁻LMX1A⁺) or hindbrain (NKX2.2⁺) in 9 DDC cultures differentiated in dSMADi+SAG− condition, and corresponding quantification of NKX2.1⁺LMX1A⁺ and NKX2.1⁻LMX1A⁺ populations (c). (d) Effect of RA concentration (50, 100, 300, 500 and 1,000 nM RA) on CYP26A1 expression in 1 DDC cultures differentiated in dSMADi-condition. (e) Temporal expression profile of CYP26A1 in cultures differentiated in dSMADi and pulsed with no RA or 300, 500 or 1,000 nM of RA for 2 days. (f) Effect of inhibition of CYP26 activity with 500 nM of the inhibitor R115866 on the expression of NKX2.1, LMX1A and PHOX2B at 9 DDC in cultures differentiated in SAG and the indicated RA conditions. (g) Relative gene expression of indicated genes in 9 DDC cultures differentiated in RA^{1D}+SAG condition and in the presence of different concentrations of the CYP26 inhibitor R115866. (h,i) Immunocytochemistry of NKX2.1, LMX1A and PHOX2B at 9 DDC in cultures differentiated in dSMADi plus the indicated conditions and in the presence or absence of 500 nM of inhibitor R115866. (j) Schematic summary of the regulatory interactions between RA and CYP26A1. (c-e) values, mean±S.D.

FIG. 4: Fast generation of functional dopaminergic neurons in in vitro cultures. (a-m) Analysis of cultures differentiated in dSMADi+RA^{2D}+SAG (a,b,e-m) or in dSMADi+SAG and indicated RA, CHIR99021 or CHIR99021+FGF8 condition (c,d). (a) Expression level changes of genes associated with floorplate identity and neurogenesis between 14 and 21 DDC cultures. (b) LMX1A and SHH immunocytochemistry in 14 and 21 DDC cultures. (c) Immunocytochemistry of neuronal marker HuCD at 17 DDC and dopaminergic neuron marker TH at 21 DDC in cultures differentiated in indicated conditions. (d) Quantification of HuCD⁺ neurons in differentiating cultures at 14, 17 and 21 DDC in indicated conditions. (e-g) Immunocytochemistry of indicated markers at 33 DDC (e,f) and 45 DDC (g). (h) Violin plot of neurite length and quantification of complexity in 30 and 40 DDC TH⁺ neurons. n=40; number of branches are represented as mean±SEM. (i) Immunocytochemistry of indicated markers in 35-40 DDC cultures. (j) Quantification of dopaminergic (TH⁺), GABAergic (GABA⁺), motor (PRPH⁺) and serotonergic (5-HT⁺) neurons in 35 DDC cultures. (k) HPLC detection of noradrenaline (NA), dopamine (DA) and serotonin (5-HT) in supernatant of 42 DDC cultures. (l) Quantification of dopamine levels in media of 42 DDC cultures in control or after KCL induced dopamine release, and in total cell lysate. Values, mean±S.D., Asterisks, Student t test, ***p<0.001. (m) Cell attached recording, showing spontaneous action potentials (top) and isolated spontaneous action potentials from cells at 40 or 45 DDC cultures (bottom). (n) Immunocytochemistry on Biocytin labelled neuron for TH at 40 DDC (top) and evoked spike train in 40 DDC neuron (bottom). (o) Schematic summary of differentiation conditions and processes timeline during dopaminergic neuron differentiation.

FIG. 5: RA-specified vMB preparations differentiate into functional dopaminergic neurons and restore motor deficits after transplantation into a rat model of PD. (a-h) Immunohistological analysis of unilaterally 6-OHDA lesioned rats seven months after grafting of vMB preparations (150.000 cells) into the striatum. (a) TH expression in striatum and substantia nigra (SN). Note TH immuno-reactivity throughout the striatum and lack of TH expression in the SN on the lesioned side (right). (b-g) Immunohistochemistry of indicated markers in grafts. (h) Quantification of net rotations per minute in rats with baseline amphetamine-induced rotation scores ≥5 ipsilateral turns per minute (n=5). (i) Rotational behavior over time after administration of amphetamine before (solid lines) and after grafting (dashed lines) of all grafted animals (n=9). (j) Preference for contralateral paw use after lesion and seven months after transplantation.

FIG. 6: Sequential treatment with RA and CHIR99021 results in the specification of caudal midbrain identity. (a) (a) Immunocytochemical analysis of ventral MB progenitor identities at 14 DDC. Cells differentiated in RA^{2D}+SAG condition express midbrain markers LMX1A and OTX2 but not caudal midbrain marker EN1. Additional treatment of cells differentiated in RA^{2D}+SAG condition with 5 μM CHIR99021 between 4-9 DDC induces EN1 in LMX1A+OTX2+ cells. Nuclei of cells visualized with DAPI staining. (b) Immunocytochemistry of dopaminergic neuron marker TH and caudal midbrain marker EN1 expression in 40 DDC cultures differentiated in indicated conditions. Many TH neurons treated with 5 μM CHI R99021 (4-9 DDC) express caudal midbrain marker EN1.

FIG. 7 (Supplementary FIG. 1):

(a) Immunocytochemistry of 4-day and 2-day cultures differentiated in dSMADi or in dSMADi+RA^{2D} for the pluripotency marker OCT4 and the neuronal markers SOX1 and PAX6. Yellow arrows (left side panel) indicate OCT4⁺/SOX1⁺ cells. Boxplot of PAX6 nuclear level (right) in 3 DDC cultures differentiated in dSMADi or dSMADi+RA^{2D}. Whiskers define 5^{th} and 95^{th} percentile. (b) Expression of genes associated with neuroectodermal, endodermal and mesodermal lineages differentiated in dSMADi+RA^{2D} condition for 2 days. (c) Western blot for OCT4 and SOX1 of 3 DDC cultures differentiated in indicated conditions. (d) Q-PCR for the markers identifying forebrain (FOXG1, SIX3), forebrain and midbrain (OTX2) regions in 9 DDC cultures differentiated in dSMADi conditions and treated with indicated RA-pulse.

FIG. 8 (Supplementary FIG. 2):

(a) Principal component analysis of 9 DDC cultures differentiated in dSMADi+SAG-condition and pulsed with RA from 0 DDC for 0 (RA^{0D}), 1 (RA^{1D}), 2 (RA^{2D}) or 4 (RA^{4D}) days.

(b) Immunocytochemistry for NKX2.1, LMX1A and PHOX2B or NKX2.2 and PHOX2B in 9 DDC cultures. Cells were differentiated in dSMADi+SAG conditions only (no RA+SAG) or together with a 2-day RA pulse (RA^{2D}+SAG) applied at different times during differentiation (0-2, 1-3, 2-4, 3-5 or 4-6 days in differentiation conditions-DDC). (c) Schematic of hESC differentiation (in dSMADi-condition) with timeline of addition of SAG to cultures (top). Immunofluorescence for LMX1A, NKX2.2 and FOXA2 (middle) and western blot for LMX1A, NKX2.2 and FOXA2 (bottom) in 9 DDC cultures treated with SAG from day 0, 1 or 2. (d) Immunofluorescence for LMX1A and FOXA2 (top) and western blot for LMX1A, FOXA2 and PAX3 (bottom) in 9 DDC cultures differentiated in dSMADi-conditions and treated from day 0 with different concentrations of SAG. (e,f) Differentiation of the hESCs lines 980 and 401, and the hiPSCs lines SM55 and SM56 in dSMADi+RA^{2D}+SAG^{0DDC}, and immunocytochemistry for the progenitor markers LMX1A, FOXA2, OTX2, Nkx2.1 and NKX6.1 in 9-day differentiating cultures (e), and for TH, FOXA2, and MAP2 in 30 DDC cultures (f).

FIG. 9 (Supplementary FIG. 3):

(a) Analysis of the expression of NKX2.1, LMX1A and PHOX2B in 9 DDC cultures differentiated in dSMADi+SAG^{0DDC} condition and pulsed with RA for 1 day or EC23 for 2 days in the presence or absence of inhibitor R115866. (b) Analysis of the expression of NKX2.1, LMX1A and PHOX2B in 9 DDC cultures differentiated in dSMADi+SAG^{0DDC} condition and pulsed with the all-trans-RA-analogues tazarotenic (TA), 13-cis-RA and 9-cis-RA for 2 days at different concentrations (10, 100, 500 and 1000 nM). (c) Analysis of the expression of NKX2.1, LMX1A and PHOX2B in 9 DDC cultures differentiated in dSMADi+SAG^{0DDC} condition and pulsed with EC23 at the indicated concentrations for 1 or 2 days.

FIG. 10 (Supplementary FIG. 4):

(a) RNA-seq data showing expression levels of floor plate genes between 9-21 DDC in cultures differentiated in dSMADi+RA^{2D}+SAG condition. (b) Immunocytochemistry for neuronal marker Tuj1 in 12 DDC culture differentiated in dSMADi+RA^{2D}+SAG condition. Nuclei visualized with DAPI. (c) TH, EN1, and GABA immunocytochemistry in 40 DDC culture. (d) Mean sodium currents of cells that showed a sodium current (whiskers cover the extend of all data points, red plus is an outlier, n=57 cells). Pie charts denote the percentage of patched cells that show a sodium current (yellow) and the percentage of cells that show no activity.

FIG. 11 (Supplementary FIG. 5):

(a) Schematic of the timeline of transplantation assay and analysis in 6-OHDA lesioned rats. (b) Immunohistological analysis of the expression of the human marker (HuNu) and dopaminergic neuron marker (TH) in grafts of all transplanted rats. (c) DAB staining of graft-derived TH⁺ neurons innervating the surrounding dorsolateral striatum (dISTR).

### EXAMPLE 1: A NOVEL RETINOIC ACID-BASED METHOD FOR RAPID AND ROBUST DERIVATION OF TRANSPLANTABLE DOPAMINE NEURONS FROM HUMAN PLURIPOTENT STEM CELLS

Significant progress has been made in directing the differentiation of human pluripotent stem cells (hPSCs) into mesencephalic dopamine (mDA) neurons for cell replacement therapy or disease modeling in Parkinson's disease (PD), but there is a continuous incentive to increase the robustness, efficiency and speed of differentiation procedures. In this study, we outline a novel retinoic acid (RA)-based method for robust and fast derivation of human mDA neurons at a high yield. The duration of RA exposure is a key determinant for a switch-like conversion of hPSCs into neural stem cells expressing a mesencephalic identity. Unlike the GSK3β inhibitor CHIR99021 commonly used to specify mesencephalic fate, the patterning response of cells to RA is remarkably tolerant to altered RA levels. Combinatorial activation of RA- and SHH signaling induces mDA neuron progenitors that initiate neurogenesis at an early time and at a high pace, and mDA neurons exhibiting functional features are attained within 40 days of culture. When transplanted into an animal model of PD, RA-specified progenitors matured into functional DA neurons that relieved motor deficits. This study provides a new approach to produce human mDA neurons that should facilitate disease modeling and drug development in vitro, and that may provide an alternative route for the generation of cells to use for cell replacement therapy of PD.

Introduction

Human pluripotent stem cells (hPSCs) in the form of embryonic stem cells (ESCs) or induced pluripotent stem cells (iPSCs) provide a scalable cellular source for the production of specific subtypes of neurons that can be utilized for high-throughput drug development, disease modeling or cell replacement therapy in neurodegenerative disorders^{1,2}. While this field is progressing rapidly, the extended time required to produce functional human neurons via stem cell differentiation in vitro provides a challenge to experimental studies and some biomedical applications³. Mesencephalic dopamine (mDA) neurons in the ventral midbrain (vMB) are of a particular interest to study due to their selective degeneration on Parkinson's disease (PD) and the potential to restore lost dopamine neurotransmission and reverse motor deficits by cell replacement therapy⁴.

Protocols for derivation of mDA neurons from hPSCs have been progressively improved, and have now, after extensive evaluation in pre-clinical grafting experiments, reached the point of clinical trials using allogeneic ESCs or autologous iPSCs as starting material². In these studies and trials, cells are transplanted as immature precursors that terminally differentiate and functionally mature in vivo over several months. Thus, although mature and functional DA neurons can be generated after transplantation, the slow differentiation and maturation of human mDA neurons also in in vitro cultures presents a challenge for the establishment of cellular platforms for disease modeling or drug development in vitro. While the yield of mDA neurons in culture has increased, the time required to obtain mature mDA neurons has remained essentially constant since the first hPSC-based protocol was described in 2004⁵. It takes ˜60 days to generate human mDA neurons exhibiting mature electrophysiological characteristics in culture^{6,7} which could reflect the minimal time required for cells to acquire mature functional features. However, single cell analyses suggest slower kinetics and less tightly controlled developmental progression of hPSC-derived mDA neurons relative to their in vivo counterparts⁸, raising the possibility that current methods have not yet been fully optimized regarding the timing of differentiation.

There is also a continuous need to further enhance the robustness of differentiation procedures in order to increase consistency between lines and to minimize batch-to-batch variability⁹. This is particularly important if working with multiple iPSC-lines in disease modeling or drug development efforts, or if considering clinical application of patient-specific autologous iPSC-lines or large numbers of human leukocyte antigen (HLA) matched donor iPSCs which may be favorable over allogeneic ESC-lines from an immunological perspective^{10,11}. In vitro derivation of mDA neurons is a multistep process in which timed activation and/or deactivation of developmental signaling pathways is used to direct the differentiation of hPSCs into progenitors with a vMB regional identity, which can differentiate into functionally mature mDA neurons in culture or after transplantation into animal models of PD^{1,12,13}. Inhibition of BMP and TGFβ signaling by a method termed dual SMAD inhibition (dSMADi) is deployed to promote a generic neural fate by preventing hPSCs from selecting alternative somatic or extraembryonic fate options¹⁴. In the absence of patterning signals NSCs acquire a cortical forebrain (FB) fate by default. Current mDA neuron protocols utilize timed activation of WNT signaling, or of WNT and FGF signaling, to specify midbrain (MB) character by mimicking the patterning activity of WNT1 and FGF8b produced by the isthmic organizer at the boundary between the MB and hindbrain (HB)¹⁵. Sonic hedgehog (SHH) signaling, in turn, is applied to ventralize cells and induce a LMX1A⁺/FOXA2⁺/OTX2⁺ vMB identity characteristic of mDA neuron progenitors^{12,13}. The glycogen synthase kinase 3β(GSK3β) inhibitor CHIR99021 is applied to activate the WNT pathway. The specification of anteroposterior (AP) identity by CHIR99021 is very precise and the patterning effect is concentration-sensitive, meaning that increased CHIR99021 leads to progressively more caudal fates¹⁶. Different cell lines respond differently to the concentration applied, which necessitates careful titrations for individual hPSC-lines⁹. As such, the development of differentiation paradigms that sidestep the reliance on precise CHIR99021 concentration to specify midbrain fate could potentially increase robustness and reduce the need for batch-to-batch adjustments. Additionally, albeit the task of generating mDA neurons can be achieved, high concentrations of CHIR99021 inhibit a broad array of kinases in addition to GSK3β¹⁷, providing another motive to consider differentiation strategies that circumvent the use of CH IR99021¹⁸.

The isthmic organizer is a secondary signaling center established after the regionalization of the rostral neural plate into brain territories has been initiated¹⁹. Early brain patterning must consequently involve signals operating upstream of WNT1 and FGF8b, and several observations implicate that the vitamin A-derivative Retinoic Acid (RA) may contribute to this process. The role of RA in patterning of the HB and spinal cord is well-established¹⁵ and it is generally assumed that RA signaling is incompatible with derivation of neurons with a more rostral origin in the CNS. RA or vitamin A is therefore actively excluded in many hPSC-based mDA neuron protocols⁹. However, surprisingly in this study, we show that a 48-hour RA pulse in combination with activation of SHH signaling is sufficient to specify hPSC-derived vMB progenitors that rapidly differentiate into functional mDA neurons in vitro, and which engraft and restore motor deficits after transplantation into a rat model of PD.

Results

A 48-Hour RA-Pulse Promotes Rapid Conversion of hPSCs into NSCs Expressing a Midbrain-Like Identity

To explore the activities of RA on hESCs directed to adopt a neural fate in response to dSMADi¹⁴, we treated cultures with 200 nM all-trans RA for the first 1, 2, 3 or 4 days of differentiation (RA^{1D}, RA^{2D}, RA^{3D}, RA^{4D}) (FIG. 1a) and monitored fate and identity of cells at different stages by immunoblotting, quantitative immunocytochemistry, qPCR or RNA-sequencing (RNA-seq). Consistent with previous studies¹⁴, analyses of the pluripotency marker OCT4 and neural-specific markers SOX1 and PAX6 revealed that cells grown in dSMADi-only conditions underwent a progressive transition from a OCT4⁺/SOX1⁻/PAX6⁻ pluripotency state into a OCT4⁻/SOX1⁺/PAX6⁺ naïve NSC-state. OCT4 was gradually downregulated and approached undetectable levels by 5 days in differentiation condition (DDC), as revealed by quantitative immunocytochemistry (FIG. 1c). Induction of SOX1 and PAX6 at low levels occurred at 3 DDC (FIG. 1b). OCT4 and SOX1 were co-expressed by cells between 3-4 DDC (FIG. 1b, c; FIG. 7a (Supplementary FIG. 1a)) showing that the conversion of hPSCs into NSCs in response to dSMADi-treatment encompass a protracted time-window over which expression of pluripotency- and neural-specific genes overlap. In contrast, in cultures treated with dSMADi and 200 nM RA for two days or longer (dSMADi+RA^{2D, 3D})(FIG. 1a), induction of SOX1 and PAX6 was observed at 2 DDC (FIG. 7a (Supplementary FIG. 1a)) and expression of OCT4 had essentially been extinguished by 3 DDC (FIG. 1b, c, f; FIG. 7a (Supplementary FIG. 1a)). Expression levels of SOX1 and PAX6 in nuclei at 3-4 DDC were also notably higher in dSMADi+RA^{2D}-cultures relative to dSMADi-only cultures (FIG. 1b, c; FIG. 7a (Supplementary FIG. 1a)). By 7 DDC, SOX1 and neural marker NESTIN were expressed at similar levels in dSMADi-only and in dSMADi+RA^{2D} cultures (FIG. 1d). RNA-seq analysis of dSMADi+RA^{2D}-cultures at 2 DDC suggested an overall downregulation of pluripotency genes and upregulation of neural lineage-specific genes (FIG. 1e). Endodermal or mesodermal lineage markers were not expressed (FIG. 7b (Supplementary FIG. 1b)). In dSMADi+RA^{2D} conditions, prompt suppression of OCT4 and fast upregulation of SOX1 was attained within a concentration range of RA between 50-500 nM (FIG. 1g). Treatment of cells with 200 nM RA for one day (dSMADi+RA^{1D}) was not sufficient to promote rapid OCT4 suppression nor fast upregulation of SOX1 (FIG. 1f), nor was treatment of cells only with RA (without dSMADi) (FIG. 7c (Supplementary FIG. 1c)). Thus, combining dSMADi treatment with exposure of hPSCs to RA for 48 hours or longer promotes a rapid and switch-like transition from pluripotency into a NSC-state.

To determine the regional identity of hPSC-derived NSCs exposed to RA for different timeframes, we analyzed cultures at 9 DDC for the expression of transcription factors whose expression alone or in combination distinguishes between FB, MB, or HB regional identities. dSMADi treatment was included in all following experiments and will not be further highlighted when describing experimental setups. As anticipated, in hPSC-cultures not exposed to RA (RA^{0D}), NSCs acquired a FOXG1⁺/OTX2⁺/HOXA2⁻ FB-like identity (FIG. 1h). A similar FB-like character was observed in RAID cultures, though the number of FOXG1⁺ cells was somewhat reduced (FIG. 1h; FIG. 7d (Supplementary FIG. 1d)). Interestingly, in RA^{2D} cultures, FB markers were suppressed and NSCs instead expressed a FOXG1⁻/OTX2⁺/HOXA2⁻ MB-like character (FIG. 1h; FIG. 7d (Supplementary FIG. 1d)). In RA^{3D} and RA^{4D} cultures, NSCs acquired a FOXG1⁻/OTX2⁻/HOXA2⁺/HOXB4⁻ rostral HB and FOXG1⁻/OTX2⁻/HOXA2⁺/HOXB4⁺ caudal HB identities, respectively (FIG. 1h). These data suggest that a 48-hour RA-pulse suppresses FB fate and imposes a MB-like identity to hPSC-derived NSCs (FIG. 1.

Combined Activation of RA and SHH Signaling Imposes a Ventral Midbrain Identity to hPSC-Derived NSCs

We next activated Shh signaling to impose a ventral identity to NSCs by treating cultures with the Smoothened agonist SAG²⁵ between 0-9 DDC, and analyzed the fate of cells exposed to RA for different timeframes by immunocytochemistry or bulk RNA-seq (FIG. 2a). At 9 DDC, NSCs generated in SAG-only or RA^{1D}+SAG conditions expressed the FB-specific markers FOXG1, SIX3, SIX6, and LHX2 (FIG. 2b) and the ventral marker NKX2.1 (FIG. 2a, b) which is a selective marker for the ventral telencephalon and diencephalon^{26,27}. In RA^{2D}+SAG cultures, FB markers were suppressed and NSCs adopted a LMX1A⁺/LMX1B⁺/FOXA2⁺/OTX2⁺ identity characteristic of vMB mDA neuron progenitors (FIG. 2a,b,d). In RA^{3D}+SAG or RA^{4D}+SAG cultures, cells expressed HOX genes and the ventral markers NKX2.2, PHOX2B, NKX6.1, and NKX6.2 typical of cranial motor neuron (MN) progenitors of the HB²⁸ (FIG. 2a, b, d; and data not shown). Principal component and hierarchal clustering analysis of RNA-seq transcriptome data showed clear segregation and broad transcriptional changes of differentially expressed genes between cells exposed to RA for different timeframes (FIG. 2c; FIG. 8 a (Supplementary FIG. 2a)). Collectively, these data show: first, that increases in the duration of RA exposure imposes progressively more caudal regional brain identities (FB->MB->HB) of hPSC-derived NSCs (FIG. 1i), and second, when combined with activation of the SHH pathway, treatment with RA for 48 h appears sufficient to impose a LMX1A⁺/FOXA2⁺/OTX2⁺ vMB-like identity to NSCs (FIG. 8e (Supplementary FIG. 2e)). Effective induction of a vMB NSC identity required the 48 hour RA-pulse to be initiated between 0-2 DDC (FIG. 8b (Supplementary FIG. 2b)) and SAG treatment to start at 0 or 1 DDC at a concentration 50 nM (FIG. 8c,d (Supplementary FIG. 2c,d)).

A LMX1A⁺/LMX1B⁺/FOXA2⁺/OTX2⁺ identity of NCSs was long considered as a molecular hallmark specific for vMB progenitors generating mDA neurons, but it was later shown that this identity is also shared by ventral progenitors in the caudal diencephalon giving rise to subthalamic nucleus (STN) neurons^{29,27} (FIG. 2d). BARHL1, BARHL2, PITX2, and NKX2.1 are selectively expressed by the STN-lineage and thus can be used to distinguish between diencephalic STN-progenitors and vMB progenitors²⁷. Analyses of RA^{2D}+SAG cultures at 14 DDC showed that the vast majority of LMX1A⁺ cells co-expressed FOXA2, OTX2, and LMX1B as well as the vMB marker CORIN³⁰ (FIG. 2g). At this stage, a subset of cells had initiated expression of NURR1 (FIG. 2g), an early marker of post-mitotic mDA neurons³¹. RNA-seq data revealed negligible expression of BARHL1, BARHL2, PITX2, and NKX2.1 (FIG. 2e) and rare LMX1A⁺ or LMX1B⁺ NSCs co-expressed NKX2.1, PITX2 or BARHL1 (FIG. 2g). Expression of NKX2.2, PHOX2B, PHOX2A, and NKX6.1 either alone or in combination define progenitors giving rise to cranial motor neurons (MNs) and serotonergic neurons (5HTNs) in the ventral HB^{28,32} or oculomotor neurons³³ and GABAergic neurons³⁴ derived lateral to mDA neurons in the MB (FIG. 2d). RNA-seq data revealed low expression of these markers in RA^{2D}+SAG cultures (FIG. 2e) and few cells expressed NKX2.2, PHOX2A, PHOX2B, and NKX6.1 at 14 DDC as determined by immunocytochemistry (FIG. 2g). Thus, the vast majority of hPSC-derived NSCs exposed to a 48-hour RA pulse and SAG express a LMX1A⁺/LMX1B⁺/FOXA2⁺/OTX2⁺ vMB identity, with little contamination of cells expressing neighboring diencephalic-, HB- or lateral MB-regional identities. Similar results were attained with two hESC-lines and two hiPSC-lines (FIG. 8e (Supplementary FIG. 2e)).

WNT1 and FGF8 signaling emanating from the isthmic organizer impose graded expression of EN1 and EN2 to the caudal MB and the rostral H B^{35,36}. WNT1, EN1, EN2 or FGF8 were expressed at very low or undetectable levels at 14 DDC (FIG. 2e). Also, the isthmic markers PAX2, PAX5, and PAX8 were expressed at negligible levels (FIG. 2e). Activation of canonical WNT signaling by CHIR99021 is associated with translocation of β-catenin into nuclei^{12,18} (FIG. 2f) and there was no accumulation of β-catenin in nuclei in response to RA treatment (FIG. 20. Together, this indicate that LMX1A⁺/FOXA2⁺/OTX2⁺ NSCs specified by RA and SAG acquire an identity reminiscent of the rostral MB, and that specification of vMB-fate occurs independently of WNT1 expression or induction of isthmic organizer-like cells in hPSC-cultures.

Self-Enhanced RA Degradation Via CYP26A1 Provides Robust vMB Patterning Response

To determine the sensitivity of the differentiation procedure to altered concentrations of RA, we cultured cells in RA^{2D}+SAG-conditions and altered the concentration of RA in the range of 100-800 nM and analyzed the identity of NSCs at 9 DDC. In cultures exposed to 200-400 nM RA, the vast majority of NSCs expressed a LMX1A⁺/NKX2.1⁻ vMB-identity and few cells expressed a diencephalic LMX1A⁺/NKX2.1⁺ identity or NKX2.2 (FIG. 3a,c). When RA concentration was reduced to 100 nM or increased to 800 nM RA, LMX1A⁺/NKX2.1⁻ NSCs were generated but at lower numbers (FIG. 3c). Accordingly, effective induction of a vMB identity is achieved within a relatively broad range of RA concentrations. When we used CHIR99221 to specify vMB identity^{9,29}, NSCs attained a LMX1A⁺/NKX2.1⁻ identity in response to 1 μM CHIR99221, but the regional identity of NSCs shifted towards a diencephalic LMX1A⁺/NKX2.1⁺ character when the concentration was reduced to 0.8 and 0.6 μM, while cells progressively adopted a NKX2.2⁺/LMX1A⁻ presumptive HB identity when the concentration was raised to 1.2 and 1.4 μM (FIG. 3b,c). These data show that specification of LMX1A⁺/NKX2.1⁻ NSCs by RA is less concentration-sensitive relative to CHIR99021, and suggests that the timeframe of RA exposure, rather than absolute RA levels, is the key parameter for vMB specification.

The CYP26 family of genes (CYP26A1, CYP26B1, CYP26C1) encode enzymes of the cytochrome p450 family that metabolize RA through oxidation³⁷. CYP26A1 is expressed by the rostral-most neuroectoderm and contributes to prevent a rostral extension of HB identity at early stages of neural development²¹. Also, in AP-patterning of the HB, negative feedback regulation of RA signaling by self-enhanced degradation via induction of CYP26 proteins is important for shaping RA gradients and to buffer for fluctuations of RA levels^{38,39}. There was a RA concentration-dependent activation and adaptive temporal expression of CYP26A1 in hPSC cultures (FIG. 3d, e). This provides a plausible mechanistic rational for the robust patterning response of cells to RA, as altered RA input can be buffered by a matching change in rate of RA turnover by CYP26A1. To explore this, we examined the fate of RA-treated hPSCs at 9 DDC after inhibiting CYP26 activity with the selective antagonist R115866⁴⁰ between 0-3 DDC. In RA^{1D}+SAG or RA^{2D}+SAG cultures treated with 500 nM R115866 cells acquired a PHOX2B⁺ HB-identity instead of a NKX2.1⁺ FB-identity or LMX1A⁺/NKX2.1⁻ vMB-identity, respectively (FIG. 3f). HOXA2 and HOXB4 were induced in these experiments suggesting a caudal HB identity (FIG. 3g; data not shown), which corresponds to a regional identity acquired after 4 days of RA exposure if CYP26 function is left intact (FIG. 1h). When the R115866 concentration was reduced to 100 nM, FB fate was suppressed but cultures contained a mix of LMX1A⁺/NKX2.1⁻ vMB cells and PHOX2B⁺ HB cells (FIG. 3g; FIG. 9a (Supplementary FIG. 3a)), presumably reflecting that a partial inhibition of CYP26A1 produces an intermediate caudalizing effect. Importantly, treatment of cells only with R115866 and SAG did not suppress NKX2.1⁺ FB-fate (FIG. 3f), establishing that the strong caudalizing effect of R115866 is RA-dependent.

Like all-trans RA, 9-cis RA, 13-cis RA and the xenobiotic RA-analogue tazarotenic acid (TA) are substrates for CYP26-mediated oxidation^{41,42}. Exposure of cells to 500 nM of these analogues for 48-hours mimicked the patterning activity of all-trans RA by imposing a LMX1A⁺/NKX2.1⁻ vMB identity (FIG. 3h; FIG. 9b (Supplementary FIG. 3b)), and inhibition of CYP26 activity resulted in a shift into a PHOX2B⁺ HB identity (FIG. 3h). The synthetic RA analogue EC23 is predicted to be resistant to CYP26 mediated oxidation⁴³ and when all-trans-RA was replaced with 200 nM of EC23, cells grown either in EC23^{1D}+SAG or EC23^{2D}+SAG conditions adopted a PHOX2B⁺ HB-identity with or without inhibition of CYP26 (FIG. 3i; FIG. 9a (Supplementary FIG. 3a)). Titration experiments showed that EC23 could induce LMX1A⁺/NKX2.1⁻ vMB cells, but this required a 20-fold reduction in concentration and treatment of cells only for 24 hours (FIG. 9c (Supplementary FIG. 3c)). Together, these data establish that the AP-patterning output in response to timed RA exposure is critically reliant on the RA concentration-dependent activation of CYP26A1 in responding hPSCs, and provides a mechanistic rationale to explain robustness and tolerance to altered RA input in the patterning process (FIG. 3j).

Fast Derivation of mDA Neurons Exhibiting Mature and Functional Properties In Vitro

A unique feature of mDA neurons is that they originate from initially non-neuronal floor plate (FP) cells at the ventral midline of the MB, and progenitors must acquire neuronal potential prior to differentiation into neurons^{30,44}. Few markers distinguish between these states, but downregulation of SHH and upregulation pro-neural bHLH proteins over time correlate with this transition⁴⁴. RNA-seq analyses of RA^{2D}+SAG treated cells isolated at 9, 12, 14 and 21 DDC showed that the expression of pan-FP markers SHH, CORIN, ARX, VTN, FERD3L, SLIT2, SULF2, and ALCAM peaked at around 12 DDC and subsequently declined (FIG. 4a; FIG. 10a (Supplementary FIG. 4a)) and immunocytochemical analyses confirmed that SHH expression was higher at 14 DDC as compared to 21 DDC (FIG. 4b). Conversely, NEUROG2, NEUROD4, and ASCL1 encoding pro-neural bHLH proteins were upregulated at 21 DDC as well as mDA neuron markers NR4A2 (NURR1) and TH, and pan-neuronal markers DCX, TUBB3, STMN2, and DLK1 (FIG. 4a). Immunocytochemical analyses revealed the presence of TuJ1⁺ neurons at 12 DDC, indicating early initiation of neurogenesis (FIG. 10b (Supplementary FIG. 4b)), and there was a progressive accumulation of HuCD⁺ neurons between 14 and 21 DDC (FIG. 4c, d). In RA^{2D}+SAG cultures at 21 DDC, ˜30% of DAPI⁺ cells accounted for HuCD⁺ neurons (FIG. 4d) and many of these had initiated expression of TH (FIG. 4c). When we instead used CHIR99021+SAG to specify LMX1A⁺/NKX2.1⁻ vMB progenitors (FIG. 3b), cells initiated neurogenesis at around 17 DDC (FIG. 4c, d) which is consistent with previous studies^{9,13}. At 21 DDC, HuCD⁺ neurons constituted ˜10% of total cells and few neurons expressed TH (FIG. 4c, d). Similar results were obtained when CHI R92211+SAG-treated cultures were complemented with FGF8b-treatment between 9-16 DDC^{9,29} (FIG. 4c). This shows that vMB progenitors specified in response to RA and SAG initiate neurogenesis at an early time and produce neurons at a high pace, indicating that cells at the population level undergo an early and relatively synchronized conversion from a FP state to a neurogenic state.

TH⁺ neurons acquired a progressively more advanced neuronal morphology with a progressive outgrowth of TH⁺ axonal processes in RA^{2D}+SAG cultures between 30-45 DDC (FIG. 4e, g, h; FIG. 8f (Supplementary FIG. 2f)). TH⁺ neurons expressed mDA neuron markers LMX1A, LMX1B, FOXA2, NURR1, and OTX2 at 30-35 DDC (FIG. 4e). A minor fraction of TH⁺ neurons had initiated expression of EN1 at 40 DDC (FIG. 10c (Supplementary FIG. 4c)) despite that RA did not induce EN1 at early progenitor stages (FIG. 2e). TH⁺ neurons also expressed the mature neuronal marker SYNAPTOPHYSIN and the monoaminergic marker VMAT2 (FIG. 40. A subset of cells expressed GIRK2 or CALBINDIN indicating the presence of both A9- and A10-like subtypes of mDA neurons⁴⁵ (FIG. 4f). At 35 DDC, ˜80% of neurons expressed TH⁺ which corresponded to ˜65% of total DAPI⁺ cells (FIG. 4i, j). ˜10% of neurons expressed GABA (FIG. 4i, j), and some of these co-expressed TH (FIG. 10c (Supplementary FIG. 4c)). Only rare neurons expressing 5-HT or the MN marker PERIPHERIN were detected (FIG. 4i, j). High performance liquid chromatography (HPLC) analyses at 42 DDC established that neurons produced and released dopamine (FIG. 4k, I) but not serotonin (5-HT) or noradrenaline (NA) (FIG. 4k). This reveals a high yield of mDA neurons with little contamination of neuronal subtypes generated in close proximity to mDA neurons in the developing brainstem. Very few cells expressed Ki67 or phospho-histone H3 at 35-40 DDC indicating low mitotic activity after long-term culturing of cells (FIG. 4i). Functional maturation of mDA neurons in vitro can be monitored by determining the time when hPSC-derived mDA neurons acquire spontaneous action potentials, evoked action potentials and voltage-dependent Na⁺ and K⁺ currents, and these traits were previously reported to be attained after ˜60 days of culture of FACS-enriched mDA neurons (25+36 days: before/after sorting) using current state-of-the-art protocols⁷. In RA^{2D}+SAG-treated hPSC-cultures, voltage-dependent Na+ and K+ currents were low at 35 DDC but increased notably at 38 DDC and remained thereafter at a largely constant level (FIG. 10d (Supplementary FIG. 4d)). Neurons showing both spontaneous (FIG. 4m) and evoked action potentials (FIG. 4n) could be recorded at 40 DDC. These data suggest that RA-based differentiation results in the generation of mDA neurons exhibiting mature functional features after 40 days of culture (FIG. 4o).

RA-Specified Cells Engraft and Reverse Motor Deficits after Transplantation into a Rat Model of PD

To determine the in vivo performance of vMB progenitors specified in response to RA^{2D}+SAG, we transplanted vMB preparations in a long-term 6-hydroxydopamine (6-OHDA) lesioned rat model of PD⁴⁶. vMB progenitors were isolated at 14 DDC (FIG. 4o) and grafted to the striata of athymic (nude) rats with prior unilateral 6-OHDA lesion to the medial forebrain bundle as previously described²⁹ (FIG. 11a (Supplementary FIG. 5a)). Seven months after transplantation, immunohistochemistry analysis showed that all 9 rats had surviving grafts with a large number of TH⁺ neurons (4300±47 TH⁺ neurons per graft, FIG. 5a) which co-labeled with the human marker HuNu (FIG. 5b-c; FIG. 11b (Supplementary FIG. 5b)). Grafted TH⁺ neurons co-expressed FOXA2, LMX1A/B, PITX3, and NURR1 (FIG. 5d-f), indicating that they adopted a mDA phenotype in vivo. A subset of these also expressed GIRK2 (FIG. 5g), a marker enriched in A9-DA neurons. The A9 identity was further supported by TH⁺ fibers innervating the surrounding dorsolateral striatum (FIG. 11c (Supplementary FIG. 5c)). The functionality of the TH⁺ neurons was assessed using amphetamine-induced rotation and paw use assessment which demonstrated complete functional recovery (FIG. 5h-j). Together, these results show that hPSC-derived vMB progenitors specified in response to RA^{2D}+SAG successfully engraft, differentiate into functional mDA neurons, and restore motor deficits in an animal model of PD to the same level and extent as cells generated via CH IR99021-based patterning^{13,29}.

Discussion

A central objective in stem cell research is the development of simple and robust differentiation techniques resulting in consistent production of desired cells at high yield⁵⁰. In this study, we outline a robust and fast method for high-yield derivation of human mDA neurons that utilizes RA to specify a MB-like character of hPSC-derived NSCs. This approach is conceptually different to the commonly used patterning via CHI R99021, as it is uncoupled from WNT signaling and since the level of caudilization is set by the duration of factor delivery rather than by concentration. Remarkably, we show that an initial 48 h RA-pulse promotes a switch-like transition from pluripotency into an NSC-state and concomitantly imposes a MB-like identity to NSCs. When combined with SHH pathway activation, vMB progenitors are induced which can rapidly differentiate into functional mDA neurons in vitro and restore dopamine neurotransmission and relieve motor deficits after transplantation into a rat model of PD at a level similar to what has been reported for other protocols^{13,29,51,52}.

The duration of RA exposure is the central parameter for vMB specification and the patterning response of cells is remarkably tolerant to altered RA concentrations, which can be attributed to a feedback mechanism termed self-enhanced decay³⁹ whereby RA regulates its own turnover rate via a concentration-dependent activation of CYP26A1. Accordingly, the fact that RA is a natural non-protein ligand subject to endogenous negative feedback regulation conveys robustness to the differentiation procedure, and renders it less sensitive to batch variations and handling, and PSC line-to-line variations, compared to patterning agents that must be supplied in precisely defined concentrations. This should reduce the need for batch-to-batch and line-to-line adjustments, and thereby greatly facilitate differentiations where multiple patient derived iPSC-lines are used^{2,53}, as well as scale-up efforts when a large number of cells are needed. Consistent with this, we obtained similar results with four distinct hPSC-lines without any adjustment to the differentiation procedure which would normally require re-titering of patterning agents⁹. It is notable that CHIR99021 is predicted to short-circuit negative feedback regulation of the WNT pathway by AXIN2⁵⁴ as it prevents degradation of β-catenin through inhibition GSK3β¹⁸, which may explain the sensitivity of vMB identity to CHIR99021 concentration. In summary, using patterning factors that operate via timed exposure rather than precise concentrations as described here opens up new possibilities for robust specification of defined neuronal subtypes for disease modeling, high-throughput drug development and cell replacement therapies.

Materials and Methods

Human PSCs Culture

Human ESCs (HS980 and 401, Karolinska Institutet) and iPSCs (SM55, SM56) were maintained on recombinant human Vitronectin (VTN) (Thermo Fisher Scientific) coated plates in iPS-Brew XF medium (Miltenyi Biotech). Cells were passaged with EDTA (0.5 mM) and ROCK inhibitor was added to the medium at a final 10 μM concentration for the first 24 h after plating. All cell lines tested negative for mycoplasma contamination.

Human PSC Differentiation

80-90% confluent PSCs cultures were rinsed twice with PBS, treated with EDTA (0.5 mM in PBS) for 5-7 min, and resuspended into single cell suspension in PBS. Cells were spin down at 400 g and resuspended in N2B27 medium (DMEM/F12: Neurobasal (1:1), 0.5×N2 and 0.5×B27 (plus vitamin A) supplements, 1×nonessential amino acids, 1% GlutaMAX, 55 μM β-mercaptoethanol-all from Thermo Fisher Scientific) containing 5 μM SB431542 (Miltenyi Biotech) and 2.5 μM DMH1 (Santa Cruz Biotech) (dual SMAD inhibition), and 10 μM ROCK inhibitor (for the first 48 hours after seeding). Cells were seeded on VTN (2 μg/cm²) and Fibronectin (FN) (2 μg/cm²) (Sigma) coated surface at a density of 60,000-80,000 cells/cm² for RA- and RA-analogues based experiments, and 20,000 cells/cm² for CHIR99021 experiments. SAG1.3 (Santa Cruz Biotechnology), CHIR99021 (Miltenyi Biotech), all-trans RA, 9-cis RA, 13-cis RA, Tazarotenic acid, R115866 (all Sigma), EC23 (Amsbio) were used at concentrations and time points described in the result section. For mDA neuron differentiation (see schematic drawing FIG. 4) neural progenitors were mechanically dissociated at 9 DDC with Stem Cell Passaging Tool (Thermo Fisher Scientific) and seeded at 1:3 ratio in N2B27 medium containing 10 μM ROCK inhibitor (for first 48 hours after dissociation) on VTN and FN coated surfaces. For terminal in vitro differentiation of dopaminergic neurons, cells were dissociated at 23 or 24 DDC with accutase (Thermo Fisher Scientific) and plated on VTN+FN+Laminin (2 μg/cm² each) (Sigma) coated surface in B27⁺ medium (Thermo Fisher Scientific) supplemented with BDNF (10 ng/ml) and GDNF (10 ng/ml) (Miltenyi Biotech), Ascorbic acid (0.2 mM) (Sigma), 10 μM ROCK inhibitor (Miltenyi Biotech) (for first 48 hours after dissociation), and 10 μM DAPT (Miltenyi Biotech). For electrophysiology an neurotransmitter content analysis cells were grown in B27 Electrophysiology medium (Thermo Fisher Scientific) supplemented with BDNF (10 ng/ml) and GDNF (10 ng/ml) (Miltenyi Biotech), Ascorbic acid (0.2 mM) (Sigma) and 10 μM ROCK inhibitor (for first 48 hours after dissociation) for at least 5 days before the experiment. The medium was routinely changed every 2-3 days.

Immunocytochemistry and Immunohistochemistry

Cells were fixed for 12 min at room temperature (RT) in 4% paraformaldehyde in PBS, rinsed 3 times in PBST (PBS with 0.1% Triton-X100), and blocked for 1 hour at RT with blocking solution (3% FCS/0.1% Triton-X100 in PBS). Cells were then incubated with primary antibodies overnight at 4° C., followed by incubation with fluorophore-conjugated secondary antibodies for 1 hour at RT. Both primary and fluorophore-conjugated secondary antibodies were diluted in blocking solution. Primary antibodies used are listed in Supplementary Table 1. Appropriate Alexa (488, 555, 647)-conjugated secondary antibodies (Molecular Probes) were used.

Immunohistochemistry was performed as described before²⁹ and primary antibodies used are listed in Supplementary Table 1.

SUPPLEMENTARY TABLE 1
List of antibodies used to characterise the differentiation process
in vitro and to validate the identity of cells after transplantation in vivo.

| | Company | Cat# | Host | Dilution |
|---|---|---|---|---|
| OCT4 | Santa Cruz | sc-9081 | rabbit | 1:1,000 |
| OCT4 | Cell signaling | 2750 | mouse | 1:2,000 |
| SOX1 | R&D Systems | AF-3369 | goat | 1:1,000 |
| ACTIN | Seven Hills | LMAB-C4 | mouse | 1:1,000 |
| | Bioreagents | | | |
| GAPDH | Invitrogen | PA 1-987 | rabbit | 1:2,000 |
| PAX6 | Sigma | HPA030775 | rabbit | 1:4,000 |
| OTX2 | R&D Systems | AF-1979 | goat | 1:2,000 |
| FOXG1 | Abeam | ab18259 | rabbit | 1:500 |
| HOXA2 | Sigma | HPA029774 | rabbit | 1:1,000 |
| HOXB4 | DSHB | 112-Hoxb4 | rat | 1:20 |
| LMXIB | Home made | | guinea-pig | 1:3,000 |
| NKX2.2 | DSHB | 74.5A5 | mouse | 1:50 |
| NKX2.1 | Abeam | ab220211 | mouse | 1:1,000 |
| LMXIA | Merck Millipore | AB10533 | rabbit | 1:4,000 |
| PHOX2B | Home made | | guinea-pig | 1:1,000 |
| FOXA2 | R&D Systems | AF-2400 | goat | 1:1,000 |
| NURRI | Santa Cruz | sc-991 | rabbit | 1:300 |
| BARHLI | Novus Biologicals | NBP1-86513 | rabbit | 1:500 |
| PITX2 | R&D Systems | AF07388 | sheep | 1:1,000 |
| NKX6.1 | DSHB | F65A2 | mouse | 1:100 |
| B-CATENIN | Santa Cruz | sc-7963 | mouse | 1:200 |
| PHOX2A | Santa Cruz | sc-81978 | mouse | 1:500 |
| ENI | DSHB | 4GII | mouse | 1:20 |
| GIRK2 | Alamone Labs | APC006 | rabbit | 1:500 |
| Tuj1 | Sigma | T8578 | mouse | 1:2,000 |
| TH | Novus Biologicals | NB300-109 | rabbit | 1:1,000 |
| TH | Novus Biologicals | NB300-110 | sheep | 1:500 |
| TH | Sigma | T2928 | mouse | 1:500 |
| TH | Pel-Freeze | P41301 | rabbit | 1:1,000 |
| 5-HT | Immunostar | 20080 | rabbit | 1:2,000 |
| CALBINDIN | Sigma | HPA023099 | rabbit | 1:5,000 |
| MAP2 | R&D Systems | MAB8304 | mouse | 1:1,000 |
| VMAT2 | Merck Millipore | AB1598P | rabbit | 1:500 |
| GABA | Sigma | A2052 | rabbit | 1:1,000 |
| Synaptophysin | Zymed | 18-0130 | rabbit | 1:1,000 |
| PITX3 | Home made | | guinea-pig | 1:2,000 |
| SHH | DSHB | 5E1 | mouse | 1:10 |
| PAX3 | DSHB | clone C2 | mouse | 1:100 |
| Ki67 | Invitrogen | 14-5698-82 | rat | 1:1,000 |
| LMXIA | Dr. M. German, San | | rabbit | 1:2,000 |
| | Francisco, CA | | | |
| PRPH | Merk Millipore | abl530 | rabbit | 1:2,000 |
| HuCD | Molecular probes | A21271 | mouse | 1:1000 |
| hNCAM | Santa Cruz | sc-106 | mouse | 1:100 |
| HuNu | Chemicon | MAB1281 | mouse | 1:200 |

Gene Expression Analyses

Total RNA was isolated using Quick-RNA Mini Prep Plus kit (Zymo Research). cDNA was prepared using Maxima First Strand cDNA synthesis kit (Thermo Fisher Scientific). Quantitative Real-Time PCR was performed in a 7500 Fast Real Time PCR system thermal cycler using Fast SYBR Green PCR Master Mix (Applied Biosystems). Analysis of gene expression was performed using the 2-ΔΔCt method, where relative gene expression was normalized to GAPDH transcript levels. Primers are listed in Supplementary Table 2.

For Illumina RNA sequencing, RNA integrity was determined on an Agilent RNA 6000 Pico chip, using Agilent 2100 BioAnalyzer (Agilent Technologies). Illumina TruSeq Stranded mRNA kit with Poly-A selection was used for library construction. Clustering was done by ‘cBot’ and samples were sequenced on NovaSeq6000 (NovaSeq Control Software 1.6.0/RTA v 3.4.4) with a 2×51 setup using ‘NovaSeqXp’ workflow in ‘51’ mode flowcell. The Bcl to FastQ conversion was performed using bcl2fastq_v2.19.1.403 from the CASAVA software suite. Reads were mapped to the human genome assembly, build GRCm38 using Tophat (v 2.0.4). Gene level abundances were estimated as FPKMs using Cufflinks (v 2.1.1)⁵⁵. Further, we processed the read count data with RNA-Seq specific function set of R package limma. The differential expression was estimated with the functions voom, ImFit, eBayes, and topTable. The variance estimates were obtained by treating all samples as replicates (design=NULL) and obtaining library sizes from counts (lib.size=NULL) without further normalization (normalize.method=“none”). The resulting fold change values of differential expression were accompanied with p-values. The latter were then adjusted for multiple testing by calculating false discovery rate (FDR) by Benjamini and Hochberg's method⁵⁶.

Heatmap plotting and PCA visualization were performed with online tools at “https://www.evinet.org/”⁵⁷ using standard parameter settings of R package heatmaply and function princomp as back end.

SUPPLEMENTARY TABLE 2
Primers used for qPCR gene
expression analysis at different
stages of neuronal differentiation.

| | | | Primer seqence |
|---|---|---|---|
| Species | Gene | Full gene name | (Fwd 5′-3′/Rev 5′-3′) |
| Hs | CYP26A1 | Cytochrome P450 | AGGAAATGACCCGCAATCTC |
| | | Family 26 | GAATGTTCTGCTCGATGCG |
| | | Subfamily A | |
| | | Member 1 | |
| Hs | F0XG1 | Forkhead box G1 | TGGCCCATGTCGCCCTTCCT |
| | | | GCCGACGTGGTGCCGTTGTA |
| Hs | GAPDH | Glyceraldehyde- | Prime Time qPCR primers |
| | | 3-Phosphate | Pre-designed IDT: |
| | | Dehydrogenase | exon: 2-3 |
| Hs | HOXA2 | Homeobox A2 | ACAGCGAAGGGAAATGTAAAAGC |
| | | | GGGCCCCAGAGACGCTAA |
| Hs | HOXB4 | Homeobox B4 | CTGGATGCGCAAAGTTCAC |
| | | | TTCCTTCTCCAGCTCCAAGA |
| Hs | LMX1A | LIM homeobox | Prime Time qPCR primers |
| | | transcription | Pre-designed IDT: |
| | | factor a | exon: 3-4 |
| Hs | NKX2.1 | NK2 homeobox 1 | AGGGCGGGGCACAGATTGGA |
| | | | GCTGGCAGAGTGTGCCCAGA |
| Hs | OTX1 | Orthodenticle | TATAAGGACCAAGCCTCATGGC |
| | | homeobox 1 | TTCTCCTCTTTCATTCCTGGGC |
| Hs | 0TX2 | Orthodenticle | ACAAGTGGCCAATTCACTCC |
| | | homeobox 2 | GAGGTGGACAAGGGATCTGA |
| Hs | PHOX2B | Paired Like | Prime Time qPCR primers |
| | | Homeobox | Pre-designed IDT: |
| | | 2B | exon: 2-3 |
| Hs | SIX3 | SIX homeobox 3 | ACCGGCCTCACTCCCACACA |
| | | | CGCTCGGTCCAATGGCCTGG |

Western Blot.

Cells were lysed in RIPA buffer (Sigma) complemented with protease and phosphatase inhibitor cocktail (ThermoFisher Scientific), and incubated on ice with shaking for 30 min. Lysate was cleared by centrifugation (20 000 g for 20 min at 4° C.) and protein concentration determined by Bicinchoninic Acid (BCA) assay. Protein lysate was resuspended in LDS buffer (Thermo Fisher Scientific) containing 2.5% 2-Mercaptoethanol and denatured at 95° C. for 5 min. 15-30 μg of protein were loaded per lane of a 4-15% SDS polyacrylamide gel (Bio-Rad) and transferred onto nitrocellulose membranes (BioRad) using a Trans-Blot Turbo System (BioRad). Membranes were incubated 1 h at RT in blocking solution (TBS with 0.1% Tween-20 (TBST) and 5% nonfat dry milk), followed by overnight incubation at 4° C. with primary antibodies. After 3 washes with TBST at RT, membranes were incubated with HRP-conjugated secondary antibodies for 1 h at RT. Detection of HRP was performed by chemiluminescent substrate SuperSignal West Dura substrate and the signal was detected on a ChemiDoc Imaging System (Bio-Rad). Primary antibodies for immunoblotting are listed in Supplementary Table 1.

HPLC

Concentrations of noradrenaline (NA), dopamine (DA) and serotonin (5-HT) in 42 DDC cultures were determined by high-performance liquid chromatography (HPLC) with electrochemical detection.

Cultures at 42 DDC differentiated in dSMADi+RA^{2D} conditions were incubated in physiological solution (140 mM NaCl; 2.5 mM KCl; 1 mM MgCl₂; 1.8 mM CaCl₂; buffered with HEPES (20 mM) at pH 7.4) or in high K⁺ solution (physiological solution with 56 mM K⁺, and the concentration of Na⁺ ions was proportionally reduced to keep the same total osmolarity). After 20 min. incubation solutions were collected and used for analysis of neurotransmitter content. To determine cellular neurotransmitter content, cells were lysed in H₂O. Incubation solutions and cellular lysates were deproteinized with 0.1M perchloric acid and after 15 min. incubation on ice, samples were double centrifuged at 20 000 g for 15 min as described before⁵⁸. Protein concentration was determined with BCA method. Samples were then analyzed in a HPLC system consisting of HTEC500 (Eicom, Kyoto, Japan), and a CMA/200 Refrigerated Microsampler (CMA Microdialysis, Stockholm, Sweden) equipped with a 20p1 loop and operating at +4° C. The potential of the glassy carbon working electrode was +450 mV vs. the Ag/AgCl reference electrode. Separation was achieved on a 200×2.0 mm Eicompak CAX column (Eicom). The mobile phase was a mixture of methanol and 0.1M phosphate buffer (pH6.0) (30:70, v/v) containing 40 mM potassium chloride and 0.13 mM EDTA-2Na. The chromatograms were recorded and integrated using the computerized data acquisition system Clarity (DataApex, The Czech Republic).

Electrophysiology.

Slides containing 35 to 60 days old neurons (n=22 slides, n=4 experiments) were placed in a recording chamber in electrophysiology medium (Neurabasal Medium, Electro; Thermo Fisher Scientific). For recording, neurons were visualized using a DIC microscope (Scientifica, Uckfield, UK) with a 60× objective (Olympus, Tokyo, Japan). Patch pipettes (resistance 3-5 MΩ for voltage clamp recordings, 5-10 MΩ for current clamp recordings), pulled on a P-87 Flaming/Brown micropipette puller (Sutter Instruments, Novato, Calif., USA), were filled with either 154 mM NaCl solution for voltage clamp recordings or 120 mM KCl solution containing 8 mM biocytin for current clamp recordings. Signals were recorded with an Axon MultiCalmp 700B amplifier and digitized at 20 kHz with an Axon Digidata 1550B digitizer (Molecular Devices, San Jose, Calif., USA). Access resistance and pipette capacitance were compensated. Cell attached voltage clamp recordings were band-pass filtered at 2 Hz low/1 kHz high and events showing an after hyperpolarization were considered spontaneous action potentials. To assess spiking patterns, neurons recorded in current clamp mode were held at a membrane potential of −60 mV. Near-threshold current steps were applied to determine the rheobase current, then 1 s current steps proportional to the rheobase current were applied. The presence of sodium currents was determined in voltage clamp mode by applying pulse with intervals of 10 mV from a holding of −60 mV. Electrical properties were extracted using a custom written Matlab (MathWorks, Natick, Mass., USA) script. After recording, slides were fixed, stained and imaged as described above. Biocytin was visualized with Streptavidin Alexa Fluor 488 (Thermo Fisher Scientific).

Graft Placement and Behavioral Analysis.

All animal procedures were performed in accordance with the European Union Directive (2010/63/EU) and were approved by the local ethical committee for the use of laboratory animals and the Swedish Department of Agriculture (Jordbruksverket). Adult female, athymic “nude” rats were purchased from Harlan/Envigo Laboratories (Hsd:RH-Foxn1rnu) and were housed as described before²⁹ with ad libitum access to food and water, under a 12-hr light/dark cycle.

All surgical procedures and lesion of the nigrostriatal pathway by unilateral injection of 6-hydroxydopamine (6-OHDA) were performed as described²⁹. Lesion severity was measured 4 weeks after 6-OHDA injection by amphetamine-induced rotations recorded over 90 min using an automated system (Omnitech Electronics)²⁹. Amphetamine-induced rotation was induced by intraperitoneal injection of 2.5 mg/kg amphetamine hydrochloride (Sigma). 4 weeks later (8 weeks after 6-OHDA lesion), animals were grafted to the striatum with a dose of 150,000 cells of hESC-derived vMB progenitors at day 14 of differentiation as previously described²⁹, and amphetamine-induced rotation was assessed 7 months after grafting.

Spontaneous paw-use asymmetry was assessed as explorative behavior in a glass cylinder as described before²⁹ 4 weeks after 6-OHDA lesion and 7 months after grafting. Paw use preference was expressed as contralateral cylinder touches as percent of total (Left/(Left+Right)×100%).

Animals were perfused after behavioral analysis and processed for immunohistochemistry.

Statistical Analyses

Unless stated otherwise, values are shown as mean±SD and asterisks in figures denote significance from Student's t test between two groups. For rotational and cylinder behavioral analysis, one-way ANOVA with Bonferroni correction and paired-sampled Student's t test were used, respectively. For all figures, *p<0.05, **p<0.01, ***p<0.001, ns=non-significant.

### EXAMPLE 2—SEQUENTIAL TREATMENT WITH RA AND CHIR99021 RESULTS IN THE SPECIFICATION OF CAUDAL MIDBRAIN IDENTITY

Mesencephalic dopamine neurons constitute several distinct subtypes, and our RA-based protocol generates enough dopaminergic neurons of the therapeutic A9-subtype to reverse motor deficits in animal models of PD. Mechanisms underlying specification of midbrain dopamine neuron subtypes remains poorly resolved, but is likely to involve sub-patterning of ventral midbrain dopaminergic progenitors along the mediolateral and rostro-caudal axes of the midbrain (Brignani, S, and Pasterkamp, R. J., Front. Neuroanat. 11, 1-18 (2017)). WNT1 signaling emanating from the isthmic organizer at the midbrain-hindbrain boundary establish polarity of midbrain progenitors along the rostro-caudal axis by inducing a caudal^{HIGH}-to-rostral^{LOW} expression gradient of the homeodomain proteins EN1 and EN2 (Wurst, W. and Bally-Cuif, L. Nat. Rev. Neuroscience 2, 99-108 (2001)). Ventral midbrain dopaminergic progenitors specified by RA^{2D}+SAG acquire a LMX1A⁺/OTX2⁺/EN1⁻ identity at 14DDC indicating a rostral midbrain character of progenitors (FIG. 6A) and a small proportion of TH⁺ dopamine neurons expressed EN1 at 40DDC (FIG. 6B). When cells grown in RA^{2D}+SAG conditions was complemented with treatment of 5 μM CHIR99021, a GSK3β kinase inhibitor used to activate WNT signaling, between 4-9DDC, a majority of LMX1A⁺/OTX2⁺ cells also expressed EN1 at 14DDC (FIG. 6A) and a large fraction of TH⁺ dopamine neurons expressed EN1 at 40DDC (FIG. 6B). This indicates that sequential treatment of differentiating hPSCs with RA and CHIR99021 can be applied to impose a more caudal LMX1A⁺/OTX2⁺/EN1⁺ identity of ventral midbrain dopamine progenitors, suggesting that minor adjustments to the basic RA^{2D} protocol can be used to sub-pattern ventral midbrain dopaminergic progenitors into different regional identities which could influence the relative proportion A9-subtypes of dopaminergic neurons generated in vitro or after transplantation in vivo.

### REFERENCES FOR EXAMPLES

1. Rowe, R. G. & Daley, G. Q. Induced pluripotent stem cells in disease modelling and drug discovery. Nat. Rev. Genet. 20, 377-388 (2019).
2. Barker, R. A., Parmar, M., Studer, L. & Takahashi, J. Human Trials of Stem Cell-Derived Dopamine Neurons for Parkinson's Disease: Dawn of a New Era. Cell Stem Cell 21, 569-573 (2017).
3. Qi, Y. et al. Combined small-molecule inhibition accelerates the derivation of functional cortical neurons from human pluripotent stem cells. Nat. Biotechnol. 35, 154-163 (2017).
4. Björklund, A. & Lindvall, 0. Replacing Dopamine Neurons in Parkinson's Disease: How did it happen? J. Parkinsons. Dis. 7, S21-S31 (2017).
5. Marton, R. M. & loannidis, J. P. A. A Comprehensive Analysis of Protocols for Deriving Dopaminergic Neurons from Human Pluripotent Stem Cells. Stem Cells Transl. Med. 8, 366-374 (2019).
6. Niclis, J. C. et al. Efficiently Specified Ventral Midbrain Dopamine Neurons from Human Pluripotent Stem Cells Under Xeno-Free Conditions Restore Motor Deficits in Parkinsonian Rodents. Stem Cells Transl. Med. 6, 937-948 (2017).
7. Riessland, M. et al. Loss of SATB1 Induces p21-Dependent Cellular Senescence in Post-mitotic Dopaminergic Neurons. Cell Stem Cell 25, 514-530.e8 (2019).
8. La Manno, G. et al. Molecular Diversity of Midbrain Development in Mouse, Human, and Stem Cells. Cell 167, 566-580.e19 (2016).
9. Nolbrant, S., Heuer, A., Parmar, M. & Kirkeby, A. Generation of high-purity human ventral midbrain dopaminergic progenitors for in vitro maturation and intracerebral transplantation. Nat. Protoc. 12, 1962-1979 (2017).
10. Wang, S. et al. Autologous iPSC-derived dopamine neuron transplantation in a nonhuman primate Parkinson's disease model. Cell Discov. 1, 1-11 (2015).
11. Morizane, A. et al. MHC matching improves engraftment of iPSC-derived neurons in non-human primates. Nat. Commun. 8, 1-12 (2017).
12. Kirkeby, A. et al. Generation of Regionally Specified Neural Progenitors and Functional Neurons from Human Embryonic Stem Cells under Defined Conditions. Cell Rep. 1, 703-714 (2012).
13. Kriks, S. et al. Dopamine neurons derived from human ES cells efficiently engraft in animal models of Parkinson's disease. Nature 480, 547-551 (2011).
14. Chambers, S. M. et al. Highly efficient neural conversion of human ES and iPS cells by dual inhibition of SMAD signaling. Nat. Biotechnol. 27, 275-80 (2009).
15. Tao, Y. & Zhang, S.-C. Neural Subtype Specification from Human Pluripotent Stem Cells. Cell Stem Cell 19, 573-586 (2016).
16. Lu, J; Zhong, X; Liu, H; Hao, L; Tzu-Ling Huang, C; Amin Sherafat, M; Jones, J; Ayala, M; Li, L; & Zhang, S. Generation of serotonin neurons from human pluripotent stem cells. Nat. Biotechnol. 34, 89-95 (2015).
17. Bain, J. et al. The selectivity of protein kinase inhibitors: a further update. Biochem. J. 408, 297-315 (2007).
18. Toledo, E. M., Gyllborg, D. & Arenas, E. Translation of WNT developmental programs into stem cell replacement strategies for the treatment of Parkinson's disease. Br. J. Pharmacol. 174, 4716-4724 (2017).
19. Gibbs, H. C., Chang-Gonzalez, A., Hwang, W., Yeh, A. T. & Lekven, A. C. Midbrain-Hindbrain Boundary Morphogenesis: At the Intersection of Wnt and Fgf Signaling. Front. Neuroanat. 11, 1-17 (2017).
25. Frank-Kamenetsky, M. et al. Small-molecule modulators of Hedgehog signaling: Identification and characterization of Smoothened agonists and antagonists. J. Biol. 1, 1-19 (2002).
26. Ericson, J., Muhr, J., Jessell, T. M. & Edlund, T. Sonic hedgehog: A common signal for ventral patterning along the rostrocaudal axis of the neural tube. Int. J. Dev. Biol. 39, 809-816 (1995).
27. Kee, N. et al. Single-Cell Analysis Reveals a Close Relationship between Differentiating Dopamine and Subthalamic Nucleus Neuronal Lineages. Cell Stem Cell 20, 29-40 (2017).
28. Pattyn, A. et al. Coordinated temporal and spatial control of motor neuron and serotonergic neuron generation from a common pool of CNS progenitors. Genes Dev. 17, 729-37 (2003).
29. Kirkeby, A. et al. Predictive Markers Guide Differentiation to Improve Graft Outcome in Clinical Translation of hESC-Based Therapy for Parkinson's Disease. Cell Stem Cell 20, 135-148 (2017).
30. Ono, Y. et al. Differences in neurogenic potential in floor plate cells along an anteroposterior location: Midbrain dopaminergic neurons originate from mesencephalic floor plate cells. Development 134, 3213-3225 (2007).
31. Zetterström, R. H. et al. Dopamine neuron agenesis in Nurr1-deficient mice. Science 276, 248-50 (1997).
32. Dias, J. M., Alekseenko, Z., Applequist, J. M. & Ericson, J. Tgfβ signaling regulates temporal neurogenesis and potency of neural stem cells in the CNS. Neuron 84, 927-39 (2014).
33. Deng, Q. et al. Specific and integrated roles of Lmx1a, Lmx1b and Phox2a in ventral midbrain development. Development 138, 3399-3408 (2011).
34. Kala, K. et al. Gata2 is a tissue-specific post-mitotic selector gene for midbrain GABAergic neurons. Development 136, 253-262 (2009).
35. Liu, A. & Joyner, A. L. Early anterior/posterior patterning of the midbrain and cerebellum. Annu. Rev. Neurosci. 24, 869-96 (2001).
36. Hynes, M. & Rosenthal, A. Specification of dopaminergic and serotonergic neurons in the vertebrate CNS. Curr. Opin. Neurobiol. 9, 26-36 (1999).
37. Thatcher, J. E. & Isoherranen, N. The role of CYP26 enzymes in retinoic acid clearance. Expert Opin. Drug Metab. Toxicol. 5, 875-86 (2009).
38. White, R. J. & Schilling, T. F. How degrading: Cyp26s in hindbrain development. Dev. Dyn. 237, 2775-2790 (2008).
39. Schilling, T. F., Nie, Q. & Lander, A. D. Dynamics and precision in retinoic acid morphogen gradients. Curr. Opin. Genet. Dev. 22, 562-569 (2012).
40. Hernandez, R. E., Putzke, A. P., Myers, J. P., Margaretha, L. & Moens, C. B. Cyp26 enzymes generate the retinoic acid response pattern necessary for hindbrain development. Development 134, 177-187 (2007).
41. Foti, R. S. et al. Identification of tazarotenic acid as the first xenobiotic substrate of human retinoic acid hydroxylase CYP26A1 and CYP26B1. J. Pharmacol. Exp. Ther. 357, 281-292 (2016).
42. White, J. A. et al. Identification of the human cytochrome P450, P450RAI-2, which is predominantly expressed in the adult cerebellum and is responsible for all-trans-retinoic acid metabolism. Proc. Natl. Acad. Sci. U.S.A 97, 6403-8 (2000).
43. Lopez-Real, R. E. et al. Application of synthetic photostable retinoids induces novel limb and facial phenotypes during chick embryogenesis in vivo. J. Anat. 224, 392-411 (2014).
44. Andersson, E. et al. Identification of intrinsic determinants of midbrain dopamine neurons. Cell 124, 393-405 (2006).
45. Reyes, S. et al. GIRK2 expression in dopamine neurons of the substantia nigra and ventral tegmental area. J. Comp. Neurol. 520, 2591-2607 (2012).
46. Grealish, S. et al. Human ESC-derived dopamine neurons show similar preclinical efficacy and potency to fetal neurons when grafted in a rat model of Parkinson's disease. Cell Stem Cell 15, 653-665 (2014).
47. Marklund, U. et al. Detailed expression analysis of regulatory genes in the early developing human neural tube. Stem Cells Dev. 23, 5-15 (2014).
48. Vadodaria, K. C., Stern, S., Marchetto, M. C. & Gage, F. H. Serotonin in psychiatry: in vitro disease modeling using patient-derived neurons. Cell Tissue Res. 371, 161-170 (2018).
49. Okaty, B. W., Commons, K. G. & Dymecki, S. M. Embracing diversity in the 5-HT neuronal system. Nat. Rev. Neurosci. 20, 397-424 (2019).
50. Zeltner, N. & Studer, L. Pluripotent stem cell-based disease modeling: current hurdles and future promise. Curr. Opin. Cell Biol. 37, 102-10 (2015).
51. Chen, Y. et al. Chemical Control of Grafted Human PSC-Derived Neurons in a Mouse Model of Parkinson's Disease. Cell Stem Cell 18, 817-26 (2016).
52. Kikuchi, T. et al. Human iPS cell-derived dopaminergic neurons function in a primate Parkinson's disease model. Nature 548, 592-596 (2017).
53. Taylor, C. J., Peacock, S., Chaudhry, A. N., Bradley, J. A. & Bolton, E. M. Generating an iPSC bank for HLA-matched tissue transplantation based on known donor and recipient hla types. Cell Stem Cell 11, 147-152 (2012).
54. Jho, E. et al. Wnt/beta-catenin/Tcf signaling induces the transcription of Axin2, a negative regulator of the signaling pathway. Mol. Cell. Biol. 22, 1172-83 (2002).
55. Trapnell, C. et al. Transcript assembly and quantification by RNA-Seq reveals unannotated transcripts and isoform switching during cell differentiation. Nat. Biotechnol. 28, 511-5 (2010).
56. Benjamini, Y. & Hochberg, Y. Controlling the False Discovery Rate: A Practical and Powerful Approach to Multiple Testing. J. R. Stat. Soc. Ser. B 57, 289-300 (1995).
57. Jeggari, A. et al. EviNet: A web platform for network enrichment analysis with flexible definition of gene sets. Nucleic Acids Res. 46, W163-W170 (2018).
58. Yang, L. & Beal, M. F. Determination of neurotransmitter levels in models of Parkinson's disease by HPLC-ECD. Methods Mol. Biol. 793, 401-15 (2011).
59. Brignani, S, and Pasterkamp, R. J., Front. Neuroanat. 11, 1-18 (2017).
60. Wurst, W. and Bally-Cuif, L. Nat. Rev. Neuroscience 2, 99-108 (2001).

1. A method for differentiating stem cells into ventral midbrain dopaminergic progenitor cells, the method comprising contacting a plurality of stem cells with an effective amount of at least one activator of retinoic acid (RA) signalling, and culturing the stem cells under conditions sufficient to cause differentiation of the stem cells into a cell population comprising ventral midbrain dopaminergic progenitor cells.

2. The method of claim 1, wherein the at least one activator of retinoic acid (RA) signalling is effective to specify ventral midbrain identity to neural stem cells

3. The method of claim 1, wherein the ventral midbrain dopaminergic progenitor cells express forkhead box protein A2 (FOXA2) and LEVI homeobox transcription factor 1 alpha (LMX1A).

4. The method of claim 1, wherein the cell population comprises at least about 50%, at least about 60%, at least about 70%, or at least about 80% ventral midbrain dopaminergic progenitor cells.

5. The method of claim 1, wherein the cell population comprises at least about 50%, at least about 60%, at least about 70%, or at least about 80% ventral midbrain dopaminergic progenitor cells at least after 7 days, such as 9-16 days, such as about 14 days, after first contacting said cell population with the at least one activator of Retinoic Acid (RA) signalling.

6. The method of claim 1, wherein the method is an in vitro method.

7. The method of claim 1, wherein the plurality of stem cell is selected from the group comprising: pluripotent stem cells; multipotent stem cells; non-embryonic stem cells such as adult stem cells (ASCs); and wherein the plurality of stem cells are derived from human, optionally wherein the human is a patient with a symptom of a neurological disorder; rodent; or primate.

8. The method of claim 1, wherein culturing the stem cells under conditions sufficient to cause differentiation of the stem cells into a cell population comprising ventral midbrain dopaminergic progenitor cells comprises contacting the stem cells with at least one activator of the Hedgehog (Hh) signalling.

9. The method of claim 1, wherein the at least one activator of the Hedgehog (Hh) signalling is selected from the group comprising: Sonic Hedgehog (SHH), Indian hedgehog (IHH), Desert hedgehog (DHH), purmorphamine, Smoothened agonists (SAGs) such as SAG 1.3 (Hh-1.3), Hh-1.2, Hh-1.4, Hh-1.5, and combinations thereof.

10. The method of claim 1, wherein culturing the stem cells under conditions sufficient to cause differentiation of the stem cells into a cell population comprising ventral midbrain dopaminergic progenitor cells comprises contacting the stem cells with at least one inhibitor of TGFβ/Activin-Nodal signalling and at least one inhibitor of bone morphogenetic protein (BMP) signalling.

11. The method of claim 10, wherein said at least one inhibitor of TGFβ/Activin-Nodal signaling is selected from the group comprising SB 431542 and SB-505124.

12. The method of claim 11, wherein said at least one inhibitor of BMP signalling is selected from the group comprising DMH-1; LDN-193189; and Noggin.

13. The method of claim 1, wherein the stem cells are contacted with the activator of retinoic acid (RA) signalling for about 1-4 days, optionally about 1-3 days.

14. The method of claim 13, wherein the at least one activator of retinoic acid (RA) signalling is not present at an effective amount after contacting the plurality of stem cells for about 1-4 days, optionally about 1-3 days.

15. The method claim 8, wherein the stem cells are contacted with the at least one activator of Hedgehog (Hh) signalling, the at least one inhibitor of TGFβ/Activin-Nodal signalling, and the at least one inhibitor of bone morphogenetic protein (BMP) signalling simultaneously with the at least one activator of retinoic acid (RA) signalling.

16. The method of claim 8, wherein the stem cells are contacted with the at least one activator of Hedgehog (Hh) signalling, the at least one inhibitor of TGFβ/Activin-Nodal signalling, and the at least one inhibitor of bone morphogenetic protein (BMP) signalling prior to being contacted with the at least one activator of retinoic acid (RA) signalling.

17. The method of claim 1, wherein the method does not comprise contacting the plurality of stem cells with an activator of wingless (Wnt) signalling simultaneously with the at least one activator of retinoic acid (RA) signalling.

18. The method of claim 1, wherein the method does not comprise contacting the plurality of stem cells with an activator of fibroblast growth factor (FGF) family signalling simultaneously with the at least one activator of retinoic acid (RA) signalling.

19. The method of claim 1, wherein the at least one activator of Retinoic Acid (RA) signalling is selected from the group comprising: a retinoic acid analogue; a RARα agonist; a RARβ agonist; a RARγ agonist; and an RXR agonist.

20. The method of claim 1, wherein the at least one activator of Retinoic Acid (RA) signalling is selected from the group comprising: retinoic acid, all-trans retinoic acid (ATRA); AM 580; TTNPB; Ch 55; CD437; BMS 961; BMS 753; AM 80; CD 2314; AC 261066; AC 55649; CD 1530; Adapalene; Tazarotenic Acid; Tazarotene; EC 19; EC23; or a functional analogue, isomer, metabolite, or derivative thereof.

21. The method of claim 1, wherein the at least one activator of Retinoic Acid (RA) signalling is selected from the group comprising: retinoic acid; and all-trans retinoic acid (ATRA), such as 9-cis RA and 13-cis RA, and Tazarotenic acid.

22. The method of claim 1, wherein the at least one activator of Retinoic Acid (RA) signalling is derived from an exogenous source.

23. The method of claim 1, wherein culturing the stem cells under conditions sufficient to cause differentiation of said stem cells to produce a cell population comprising ventral midbrain dopaminergic progenitor cells takes place in a two-dimensional and/or three-dimensional cell culture.

24. The method of claim 1, wherein the cell population comprises a therapeutically effective amount of ventral midbrain dopaminergic progenitor cells.

25. The method of claim 1, further comprising differentiating the population comprising ventral midbrain dopaminergic progenitor cells into mesencephalic dopaminergic neurons.

26. The method of claim 25, wherein the mesencephalic dopaminergic neurons express one or more of forkhead box protein A2 (FOXA2), LIM homeobox transcription factor 1 alpha (LMX1A), LIM homeobox transcription factor 1 beta (LMX1B), Orthodenticle homeobox 2 (OTX2), Nuclear receptor related 1 (NURR1); Paired Like Homeodomain 3 (PITX3), GIRK2, vesicular monoamine transporter (VMAT2), synaptophysin, and Tyrosine hydroxylase (TH).

27. The method of claim 25, wherein the population comprising differentiated mesencephalic dopaminergic neurons is obtainable within about 30-40 days after first contacting the plurality of stem cells with the at least one activator of Retinoic Acid (RA) signalling.

28. The method of claim 25, wherein within about 30-40 days after first contacting the plurality of stem cells with the at least one activator of Retinoic Acid (RA) signalling, the total neuronal cell population comprises at least 70%, such as at least 80%, or at least 90% mesencephalic dopaminergic neurons.

29. A method of screening for a candidate drug comprising (a) providing a population of ventral midbrain dopaminergic progenitor cells obtainable or obtained by claim 1, or providing a population of differentiated mesencephalic dopaminergic neurons obtainable or obtained by claim 25 (b) contacting the population with a candidate drug; and (c) determining the effect of the candidate drug on the cell population.

30. A method for providing an enriched population of:
i. ventral midbrain dopaminergic progenitor cells, wherein the method comprises carrying out the method as defined in any of claim 1; or
ii. differentiated midbrain dopaminergic (DA) neurons, wherein the method comprises carrying out the method as defined in claim 25.

31. A neuronal cell population comprising a therapeutically effective amount of ventral midbrain dopaminergic progenitor cells obtained or obtainable by a method according to claim 1, optionally wherein at least 60%, such as at least 65%, such as at least 70%, such as at least 75%, or at least 80% of the cell population are ventral midbrain dopaminergic progenitor cells.

32. The neuronal cell population of claim 31, wherein at least about 80% of the cell population express forkhead box protein A2 (FOXA2), LIM homeobox transcription factor 1 alpha (LMX1A), LIM homeobox transcription factor 1 beta (LMX1B) and Orthodenticle homeobox 2 (OTX2).

33. A differentiated cell population comprising a therapeutically effective amount of mesencephalic dopaminergic neurons obtained or obtainable by a method according to claim 25, optionally wherein at least 60%, such as at least 65%, such as at least 70%, such as at least 75%, or at least 80% of the total cells are mesencephalic dopaminergic neurons.

34. Use of at least one activator of Retinoic Acid (RA) signalling for differentiating stem cells into ventral midbrain dopaminergic progenitor cells.

35. The use of claim 34, wherein differentiating stem cells into ventral midbrain dopaminergic progenitor cells is as defined in claim 1.

36. An isolated cell population, comprising a therapeutically effective amount of ventral midbrain dopaminergic progenitor cells.

37. The isolated cell population of claim 36, wherein at least about 80% of the cell population express forkhead box protein A2 (FOXA2), LIM homeobox transcription factor 1 alpha (LMX1A), LIM homeobox transcription factor 1 beta (LMX1B) and Orthodenticle homeobox 2 (OTX2).

38. A pharmaceutical composition comprising a cell population comprising a therapeutically effective amount of ventral midbrain dopaminergic progenitor cells of claim 36, and/or obtained or obtainable by the method of claim 1, for use in medicine.

39. The pharmaceutical composition of claim 38, further comprising a pharmaceutically acceptable carrier, diluent and/or excipient.

40. The pharmaceutical composition of claim 38, formulated for transplantation.

41. A kit for differentiating a plurality of stem cells into ventral midbrain dopaminergic progenitor cells or into mesencephalic dopaminergic neurons in vitro, comprising:
at least one activator Retinoic Acid (RA) signalling;
at least one activator of Sonic Hedgehog (SHH) signalling;
at least one inhibitor of TGFβ/Activin-Nodal signalling; and/or
at least one inhibitor of bone morphogenetic protein (BMP) signalling.

42. The kit of claim 41, wherein the kit further comprises a plurality of markers of ventral midbrain dopaminergic progenitor cells or mesencephalic dopaminergic neurons.

43. A kit comprising a therapeutically effective amount of ventral midbrain dopaminergic progenitor cells obtained or obtainable by the method of claim 1 and one or more dopaminergic neuron lineage specific activators and/or inhibitors.

44. A cell population comprising a therapeutically effective amount of ventral midbrain dopaminergic progenitor cells of claim 36, and/or obtained or obtainable by the method of claim 1, for use in medicine.

45. A cell population comprising a therapeutically effective amount of ventral midbrain dopaminergic progenitor cells of claim 36, and/or obtained or obtainable by the method of claim 1, for use in treating or preventing neurodegeneration in a subject and/or a disease and/or condition characterized by the loss of midbrain dopaminergic neurons in a subject.

46. A method for treating or preventing neurodegeneration in a subject and/or a disease and/or condition characterized by the loss of midbrain dopaminergic neurons in a subject, comprising administering to the subject a cell population comprising a therapeutically effective amount of ventral midbrain dopaminergic progenitor cells of claim 36, and/or obtained or obtainable by the method of claim 1, in an amount effective to treat or prevent the neurodegeneration in the subject and/or a disease and/or condition characterized by the loss of midbrain dopaminergic neurons in a subject.

47. Use of a cell population comprising a therapeutically effective amount of ventral midbrain dopaminergic progenitor cells of claim 36, and/or obtained or obtainable by the method of claim 1, for the manufacture of a medicament for treating or preventing neurodegeneration in a subject and/or a disease and/or condition characterized by the loss of midbrain dopaminergic neurons in a subject.

48. The cell population for use of claim 44, wherein the subject exhibits at least one neurological symptom, wherein the neurological symptom is selected from the group comprising of: resting tremor, rigidity, bradykinesia (slow movement), and postural instability and/or impaired balance and coordination.

49. The cell population for use, method, or use of claim 48, wherein said subject shows a reduction of at least one of said neurological symptom.

50. The cell population for use of claim 44 wherein the population comprising ventral midbrain dopaminergic progenitor cells is administered by transplantation to a subject under conditions that allow in vivo engraftment of the population of cells.

51. A cell population comprising a therapeutically effective amount of ventral midbrain dopaminergic progenitor cells of claim 36, for use in transplanting into a subject in need thereof.

52. The cell population for use of any of claim 44, wherein the subject has or is at risk of a neurodegenerative disease selected from the group comprising: Parkinson's disease, Parkinsonism syndrome, Alzheimer's disease, stroke, amyotrophic lateral sclerosis, Binswanger's disease, Huntington's chorea, multiple sclerosis, myasthenia gravis and Pick's disease.

53. A method of differentiating, a population for use, use of a population, a method of treating, or a kit substantially as described herein, with reference to the accompanying description, examples and drawings.
